(19) United States
(12) Patent Application Publication
(10) Pub. No.: US 2024/0189272 A1
(43) Pub. Date: Jun. 13, 2024

(54) FORMULATIONS AND COMPOSITIONS OF DOCETAXEL

(71) Applicant: Zhuhai Beihai Biotech Co., Ltd., Jinwan (CN)

(72) Inventor: Qun Sun, Princeton, NJ (US)

(21) Appl. No.: 18/387,327

(22) Filed: Nov. 6, 2023

Related U.S. Application Data
(63) Continuation of application No. 16/609,592, filed on Oct. 30, 2019.
(60) Provisional application No. 62/492,714, filed on May 1, 2017.

(51) Int. Cl. A61K 31/337; A61K 9/08; A61K 9/14; A61K 38/38; A61K 47/12

(52) U.S. Cl. CPC A61K 31/337; A61K 9/08; A61K 9/14; A61K 38/385; A61K 47/12

(57) ABSTRACT

This document relates to a composition comprising docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate. This document also relates to a composition consisting essentially of docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate. This document also relates to a composition comprising docetaxel, proteins comprising a human serum albumin, and sodium N-acetyltryptophanate, wherein the docetaxel and the human serum albumin in the composition have a ratio by weight from about 1:80 to less than 1:150.

### CLAIM OF PRIORITY

This application is a continuation and claims the benefit of U.S. application Ser. No. 16/609,592, filed Oct. 30, 2019, which is the national stage of international Application No. PCT/US2018/030489, filed May 1, 2018, which claims the benefit of U.S. provisional application No. 62/492,714 filed May 1, 2017. The entire contents of the foregoing are hereby incorporated by reference.

### TECHNICAL FIELD

This document relates to formulations and compositions for the treatment of proliferative diseases, and more particularly to formulations and compositions comprising docetaxel and human serum albumin.

### BACKGROUND

Many drugs for parenteral use are insoluble in water, and are thus formulated with solubilizing agents, surfactants, solvents, and/or emulsifiers that are irritating, allergenic, or toxic when administered to patients. See. e.g., Briggs et al., Anesthesis 37, 1099 (1982), and Waugh et al., Am. J. Hosp. Pharmacists, 48, 1520 (1991)). Further, many of these drugs, especially those administered intravenously, cause undesirable side effects such as venous irritation, phlebitis, burning and pain on injection, venous thrombosis, extravasation, and other administration related side effects. Additionally, often free drugs present in formulations induce pain or irritation upon administration.

Taxanes play an important role in the treatment of various solid tumors. As a second-generation semi-synthetic taxane derivative, docetaxel is about twice as potent as paclitaxel in inhibiting microtubule depolymerization, and has the unique ability to alter certain classes of microtubules, which differs from most spindle poisons currently used in clinic. However, docetaxel has very poor water solubility. The clinical intravenous administration of commercially available Docetaxel (Taxotere®) is formulated in a highly concentrated solution containing 40 mg Docetaxel and 1040 mg Polysorbate 80 per mL. This concentrated solution must be carefully diluted with solvent containing 13% ethanol in saline before administration, and must be used within 4 hours due to its limited stability. These attributes limit the administration of docetaxel. Further, it has been reported that docetaxel administration is associated with the occurrence of unpredictable (acute) hypersensitivity reactions and cumulative fluid retention. See, e.g., Trudeau M E et al., J Clin Oncol 1996; 14:422-8, Piccart M J et al., J Natl Cancer Inst 1995; 87:676-81, Bruno R et al., J Clin Oncol 1998; 16:187-96. These side-effects have been attributed, in part, to the presence of polysorbate 80.

US 2005/0282734 describes complexes of paclitaxel and albumin. Successful formulations described in this document require acidic pH.

WO 2014/121033 describes complexes of camptothecin and albumin.

US 2012/0076862 describes nanoparticles of taxane and albumin.

US 2010/0076008 describes paclitaxel non-covalently bound to HSA.

WO 2016/187147 describes complexes and compositions of docetaxel and HSA.

Accordingly, there is a need in the art for stable and non-toxic formulations of docetaxel. The compositions and methods described in the present application help meet this need.

### SUMMARY

Provided herein is a composition comprising docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate. In some embodiments, at least 96% of the proteins in the composition is human serum albumin. In some embodiments, the pH of a composition is neutral (e.g., pH of the composition is from about 5 to about 8, from about 5.5 to about 7.5, or from about 6 to about 7.5, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the docetaxel and the human serum albumin in the composition are in a ratio by weight from about 1:70 to about 1:250. In some embodiments, the docetaxel and the human serum albumin in the composition are in a ratio by weight from about 1:80 to about 1:200. In some embodiments, the docetaxel and the human serum albumin in the composition are in a ratio by weight from about 1:90 to about 1:200. In some embodiments, the docetaxel and the human serum albumin in the composition are in a ratio by weight from about 1:95 to about 1:200. In some embodiments, the docetaxel and the human serum albumin in the composition are in a ratio by weight from about 1:100 to about 1:200. In some embodiments, the docetaxel and the human serum albumin in the composition are in a ratio by weight from about 1:80 to about 1:180. In some embodiments, the docetaxel and the human serum albumin in the composition are in a ratio by weight from about 1:90 to about 1:180. In some embodiments, the docetaxel and the human serum albumin in the composition are in a ratio by weight from about 1:100 to about 1:180. In some embodiments, the docetaxel and the human serum albumin in the composition are in a ratio by weight from about 1:80 to about 1:150. In some embodiments, the docetaxel and the human serum albumin in the composition are in a ratio by weight from about 1:90 to about 1:150. In some embodiments, the docetaxel and the human serum albumin in the composition are in a ratio by weight from about 1:95 to about 1:150. In some embodiments, the docetaxel and the human serum albumin in the composition are in a ratio by weight from about 1:100 to about 1:150. In some embodiments, the docetaxel and the human serum albumin in the composition are in a ratio by weight from about 1:80 to about 1:140. In some embodiments, the docetaxel and the human serum albumin in the composition are in a ratio by weight from about 1:90 to about 1:140. In some embodiments, the docetaxel and the human serum albumin in the composition are in a ratio by weight from about 1:95 to about 1:140. In some embodiments, the docetaxel and the human serum albumin in the composition are in a ratio by weight from about 1:100 to about 1:140. In some embodiments, the docetaxel and the human serum albumin in the composition are in a ratio by weight from about 1:80 to about 1:130. In some embodiments, the docetaxel and the human serum albumin in the composition are in a ratio by weight from about 1:90 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:100 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:95 to about 1:125. In some embodiments, the docetaxel and the human serum albumin in the composition are in a ratio by weight from about 1:100 to about 1:125. In some embodiments, the docetaxel and the human serum albumin in the composition are in a ratio by weight from about 1:90 to about 1:120. In some embodiments, the docetaxel and the human serum albumin in the composition are in a ratio by weight from about 1:100 to about 1:120. In some embodiments, the docetaxel and the human serum albumin in the composition are in a ratio by weight from about 1:90 to about 1:110. In some embodiments, the docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:170, about 1:180, about 1:190, about 1:200, about 1:210, about 1:220, or about 1:250. In some embodiments, the docetaxel and the human serum albumin in the composition are in a ratio by weight of about 1:90. In some embodiments, the docetaxel and the human serum albumin in the composition are in a ratio by weight of about 1:95. In some embodiments, the docetaxel and the human serum albumin in the composition are in a ratio by weight of about 1:100. In some embodiments, the docetaxel and the human serum albumin in the composition are in a ratio by weight of about 1:105. In some embodiments, the docetaxel and the human serum albumin in the composition are in a ratio by weight of about 1:110. In some embodiments, the docetaxel and the human serum albumin in the composition are in a ratio by weight of about 1:115. In some embodiments, the docetaxel and the human serum albumin in the composition are in a ratio by weight of about 1:120. In some embodiments, the docetaxel and the human serum albumin in the composition are in a ratio by weight of about 1:125. In some embodiments, the docetaxel and the human serum albumin in the composition are in a ratio by weight of about 1:130. In some embodiments, the docetaxel and the human serum albumin in the composition are in a ratio by weight of about 1:135. In some embodiments, the docetaxel and the human serum albumin in the composition are in a ratio by weight of about 1:140.

In some embodiments, the human serum albumin is a native human serum albumin. In some embodiments, the human serum albumin is a native human serum albumin obtained from pools of human plasma. In some embodiments, the human serum albumin is a native human serum albumin obtained from pools of human plasma by low temperature controlled fractionation according to the Cohn process.

In some embodiments, the ratio of the amount of sodium caprylate to the amount of the proteins in the composition is from about 0.100 mmol to about 0.200 mmol of sodium caprylate per one gram of the proteins. In some embodiments, the ratio of the amount of sodium caprylate to the amount of the proteins in the composition is from about 0.120 mmol to about 0.190 mmol of sodium caprylate per one gram of the proteins. In some embodiments, the ratio of the amount of sodium caprylate to the amount of the proteins in the composition is from about 0.140 mmol to about 0.180 mmol of sodium caprylate per one gram of the proteins. In some embodiments, the ratio of the amount of sodium caprylate to the amount of the proteins in the composition is from about 0.145 mmol to about 0.175 mmol of sodium caprylate per one gram of the proteins. In some embodiments, the ratio of the amount of sodium caprylate to the amount of the proteins in the composition is from about 0.150 mmol to about 0.170 mmol of sodium caprylate per one gram of the proteins. In some embodiments, the ratio of the amount of sodium caprylate to the amount of the proteins in the composition is about 0.160 mmol of sodium caprylate per one gram of the proteins. In some embodiments, the ratio of the amount of sodium caprylate to the amount of the proteins in the composition is about 0.155 mmol of sodium caprylate per one gram of the proteins. In some embodiments, the ratio of the amount of sodium caprylate to the amount of the proteins in the composition is about 0.165 mmol of sodium caprylate per one gram of the proteins.

In some embodiments, the composition is a solid formulation. In some embodiments, the composition is an aqueous formulation. In some embodiments, the aqueous formulation is substantially free of solvent other than water. In some embodiments, the aqueous formulation is a clear aqueous solution. In some embodiments, the aqueous formulation is free of surfactants selected from the group selected from CREMOPHOR® surfactants and Polysorbate 80. In some embodiments, the pH of the solid formulation or the aqueous formulation is neutral (e.g., pH of the composition is from about 5 to about 8, from about 5.5 to about 7.5, or from about 6 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

Also, provided herein is a pharmaceutical composition comprising the composition comprising docetaxel, proteins comprising a human serum albumin, and sodium caprylate as described herein, and a pharmaceutically acceptable carrier.

Also, provided herein is a method of treating cancer comprising the step of administering to a subject in need thereof a therapeutically effective amount of a pharmaceutical composition comprising the composition comprising docetaxel, proteins comprising a human serum albumin, and sodium caprylate as described herein, and a pharmaceutically acceptable carrier.

In some embodiments, the cancer is a solid tumor cancer. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma. In some embodiments, the cancer is a breast cancer. In some embodiments, the cancer is a non-small cell lung cancer. In some embodiments, the cancer is a prostate cancer. In some embodiments, the cancer is a gastric cancer. In some embodiments, the cancer is a head and neck cancer. In some embodiments the cancer is an ovarian cancer. In some embodiments, the cancer is a pancreatic cancer. In some embodiments, the cancer is a Kaposi's sarcoma.

Also, provided herein is a composition consisting essentially of docetaxel, proteins comprising a human serum albumin and sodium caprylate, wherein the docetaxel and the human serum albumin in the composition have a ratio by weight from about 1:70 to about 1:300, and wherein the composition does not contain sodium N-acetyltryptophanate. In some embodiments, at least 96% of the proteins in the composition is human serum albumin. In some embodiments, the pH of a composition is neutral (e.g., pH of the composition is from about 5 to about 8, from about 5.5 to about 7.5, or from about 6 to about 7.5, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

Also provided herein is a composition comprising docetaxel, proteins comprising a human serum albumin, and sodium N-acetyltryptophanate, wherein the docetaxel and the human serum albumin in the composition have a ratio by weight from about 1:80 to less than 1:150. In some embodiments, at least 96% of the proteins in the composition is human serum albumin. In some embodiments, the pH of a composition is neutral (e.g., pH of the composition is from about 5 to about 8, from about 5.5 to about 7.5, or from about 6 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the docetaxel and the human serum albumin in the composition are in a ratio by weight from about 1:85 to less than 1:150. In some embodiments, the docetaxel and the human serum albumin in the composition are in a ratio by weight from about 1:90 to less than 1:150. In some embodiments, the docetaxel and the human serum albumin in the composition are in a ratio by weight from about 1:100 to less than 1:150. In some embodiments, the docetaxel and the human serum albumin in the composition are in a ratio by weight from about 1:95 to less than 1:150. In some embodiments, the docetaxel and the human serum albumin in the composition are in a ratio by weight from about 1:110 to less than 1:150. In some embodiments, the docetaxel and the human serum albumin in the composition are in a ratio by weight from about 1:80 to about 1:145. In some embodiments, the docetaxel and the human serum albumin in the composition are in a ratio by weight from about 1:90 to about 1:145. In some embodiments, the docetaxel and the human serum albumin in the composition are in a ratio by weight from about 1:100 to about 1:145. In some embodiments, the docetaxel and the human serum albumin in the composition are in a ratio by weight from about 1:95 to about 1:145. In some embodiments, the docetaxel and the human serum albumin in the composition are in a ratio by weight from about 1:110 to about 1:145. In some embodiments, the docetaxel and the human serum albumin in the composition are in a ratio by weight from about 1:80 to about 1:140. In some embodiments, the docetaxel and the human serum albumin in the composition are in a ratio by weight from about 1:90 to about 1:140. In some embodiments, the docetaxel and the human serum albumin in the composition are in a ratio by weight from about 1:100 to about 1:140. In some embodiments, the docetaxel and the human serum albumin in the composition are in a ratio by weight from about 1:95 to about 1:140. In some embodiments, the docetaxel and the human serum albumin in the composition are in a ratio by weight from about 1:110 to about 1:140. In some embodiments, the docetaxel and the human serum albumin in the composition are in a ratio by weight from about 1:80 to about 1:135. In some embodiments, the docetaxel and the human serum albumin in the composition are in a ratio by weight from about 1:90 to about 1:135. In some embodiments, the docetaxel and the human serum albumin in the composition are in a ratio by weight from about 1:95 to less than 1:135. In some embodiments, the docetaxel and the human serum albumin in the composition are in a ratio by weight from about 1:100 to about 1:135. In some embodiments, the docetaxel and the human serum albumin in the composition are in a ratio by weight from about 1:80 to about 1:130. In some embodiments, the docetaxel and the human serum albumin in the composition are in a ratio by weight from about 1:85 to about 1:130. In some embodiments, the docetaxel and the human serum albumin in the composition are in a ratio by weight from about 1:90 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:100 to about 1:130. In some embodiments, the docetaxel and the human serum albumin in the composition are in a ratio by weight from about 1:80 to about 1:125. In some embodiments, the docetaxel and the human serum albumin in the composition are in a ratio by weight from about 1:85 to about 1:125. In some embodiments, the docetaxel and the human serum albumin in the composition are in a ratio by weight from about 1:90 to about 1:125. In some embodiments, the docetaxel and the human serum albumin in the composition are in a ratio by weight from about 1:95 to about 1:125. In some embodiments, the docetaxel and the human serum albumin in the composition are in a ratio by weight from about 1:100 to about 1:125. In some embodiments, the docetaxel and the human serum albumin in the composition are in a ratio by weight from about 1:80 to about 1:120. In some embodiments, the docetaxel and the human serum albumin in the composition are in a ratio by weight from about 1:90 to about 1:120. In some embodiments, the docetaxel and the human serum albumin in the composition are in a ratio by weight from about 1:95 to about 1:120. In some embodiments, the docetaxel and the human serum albumin in the composition are in a ratio by weight from about 1:100 to about 1:120. In some embodiments, the docetaxel and the human serum albumin in the composition are in a ratio by weight from about 1:80 to about 1:110. In some embodiments, the docetaxel and the human serum albumin in the composition are in a ratio by weight from about 1:90 to about 1:110. In some embodiments, the docetaxel and the human serum albumin in the composition are in a ratio by weight from about 1:90 to about 1:115. In some embodiments, the docetaxel and the human serum albumin in the composition are in a ratio by weight from about 1:90 to about 1:100. In some embodiments, the docetaxel and the human serum albumin in the composition are in a ratio by weight from about 1:100 to about 1:110. In some embodiments, the docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, or about 1:145. In some embodiments, the docetaxel and the human serum albumin in the composition are in a ratio by weight of about 1:90. In some embodiments, the docetaxel and the human serum albumin in the composition are in a ratio by weight of about 1:95. In some embodiments, the docetaxel and the human serum albumin in the composition are in a ratio by weight of about 1:100. In some embodiments, the docetaxel and the human serum albumin in the composition are in a ratio by weight of about 1:105. In some embodiments, the docetaxel and the human serum albumin in the composition are in a ratio by weight of about 1:110. In some embodiments, the docetaxel and the human serum albumin in the composition are in a ratio by weight of about 1:115. In some embodiments, the docetaxel and the human serum albumin in the composition are in a ratio by weight of about 1:120.

In some embodiments, the human serum albumin is a native human serum albumin. In some embodiments, the human serum albumin is a native human serum albumin obtained from pools of human plasma. In some embodiments, the human serum albumin is a native human serum albumin obtained from pools of human plasma by low temperature controlled fractionation according to the Cohn process.

In some embodiments, the composition contains both sodium N-acetyltryptophanate and sodium caprylate. In some embodiments, the composition contains both sodium N-acetyltryptophanate and sodium caprylate in a molar ratio of about 1:1. In some embodiments, the composition contains only sodium N-acetyltryptophanate with no sodium caprylate.

In some embodiments, the composition comprises from about 0.064 mmol to about 0.096 mmol sodium N-acetyltryptophanate and from about 0.064 mmol to about 0.096 mmol sodium caprylate per gram of the proteins in the composition. In some embodiments, the composition comprises from about 0.064 mmol to about 0.096 mmol sodium N-acetyltryptophanate per gram of the proteins in the composition. In some embodiments, the composition comprises from about 0.064 mmol to about 0.096 mmol sodium caprylate per gram of the proteins in the composition. In some embodiments, the composition comprises from about 0.07 mmol to about 0.09 mmol sodium N-acetyltryptophanate and from about 0.07 mmol to about 0.09 mmol sodium caprylate per gram of the proteins in the composition. In some embodiments, the composition comprises from about 0.07 mmol to about 0.09 mmol sodium N-acetyltryptophanate per gram of the proteins in the composition. In some embodiments, the composition comprises from about 0.07 mmol to about 0.09 mmol sodium caprylate per gram of the proteins in the composition. In some embodiments, the composition comprises about 0.08 mmol sodium N-acetyltryptophanate and about 0.08 mmol sodium caprylate per gram of the proteins in the composition. In some embodiments, the composition comprises about 0.08 mmol sodium N-acetyltryptophanate per gram of the proteins in the composition. In some embodiments, the composition comprises about 0.08 mmol sodium caprylate per gram of the proteins in the composition.

In some embodiments, the composition is a solid formulation. In some embodiments, the composition is an aqueous formulation. In some embodiments, the aqueous formulation is substantially free of solvent other than water. In some embodiments, the aqueous formulation is a clear aqueous solution. In some embodiments, the aqueous formulation is free of surfactants selected from the group selected from CREMOPHOR® surfactants and Polysorbate 80. In some embodiments, the pH of the solid formulation or the aqueous formulation is neutral (e.g., pH of the composition is from about 5 to about 8, from about 5.5 to about 7.5, or from about 6 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

Also, provided herein is a pharmaceutical composition comprising the composition comprising docetaxel, proteins comprising a human serum albumin and N-acetyltryptophanate as described herein, and a pharmaceutically acceptable carrier.

Also, provided herein is a method of treating cancer comprising the step of administering to a subject in need thereof a therapeutically effective amount of a pharmaceutical composition comprising the composition comprising docetaxel, proteins comprising a human serum albumin and N-acetyltryptophanate as described herein, and a pharmaceutically acceptable carrier.

In some embodiments, the cancer is a solid tumor cancer. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma. In some embodiments, the cancer is a breast cancer. In some embodiments, the cancer is a non-small cell lung cancer. In some embodiments, the cancer is a prostate cancer. In some embodiments, the cancer is a gastric cancer. In some embodiments, the cancer is a head and neck cancer. In some embodiments the cancer is an ovarian cancer. In some embodiments, the cancer is a pancreatic cancer. In some embodiments, the cancer is a Kaposi's sarcoma.

### DETAILED DESCRIPTION

Provided herein is a composition comprising docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate. In some embodiments, at least 96% of the proteins in the composition is human serum albumin. In some embodiments, the pH of a composition is neutral (e.g., pH of the composition is from about 5 to about 8, from about 5.5 to about 7.5, or from about 6 to about 7.5, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the docetaxel and the human serum albumin in the composition are in a ratio by weight from about 1:70 to about 1:250. In some embodiments, the docetaxel and the human serum albumin in the composition are in a ratio by weight from about 1:80 to about 1:200. In some embodiments, the docetaxel and the human serum albumin in the composition are in a ratio by weight from about 1:90 to about 1:200. In some embodiments, the docetaxel and the human serum albumin in the composition are in a ratio by weight from about 1:95 to about 1:200. In some embodiments, the docetaxel and the human serum albumin in the composition are in a ratio by weight from about 1:100 to about 1:200. In some embodiments, the docetaxel and the human serum albumin in the composition are in a ratio by weight from about 1:80 to about 1:180. In some embodiments, the docetaxel and the human serum albumin in the composition are in a ratio by weight from about 1:90 to about 1:180. In some embodiments, the docetaxel and the human serum albumin in the composition are in a ratio by weight from about 1:100 to about 1:180. In some embodiments, the docetaxel and the human serum albumin in the composition are in a ratio by weight from about 1:80 to about 1:150. In some embodiments, the docetaxel and the human serum albumin in the composition are in a ratio by weight from about 1:90 to about 1:150. In some embodiments, the docetaxel and the human serum albumin in the composition are in a ratio by weight from about 1:95 to about 1:150. In some embodiments, the docetaxel and the human serum albumin in the composition are in a ratio by weight from about 1:100 to about 1:150. In some embodiments, the docetaxel and the human serum albumin in the composition are in a ratio by weight from about 1:80 to about 1:140. In some embodiments, the docetaxel and the human serum albumin in the composition are in a ratio by weight from about 1:90 to about 1:140. In some embodiments, the docetaxel and the human serum albumin in the composition are in a ratio by weight from about 1:95 to about 1:140. In some embodiments, the docetaxel and the human serum albumin in the composition are in a ratio by weight from about 1:100 to about 1:140. In some embodiments, the docetaxel and the human serum albumin in the composition are in a ratio by weight from about 1:80 to about 1:130. In some embodiments, the docetaxel and the human serum albumin in the composition are in a ratio by weight from about 1:90 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:100 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:95 to about 1:125. In some embodiments, the docetaxel and the human serum albumin in the composition are in a ratio by weight from about 1:100 to about 1:125. In some embodiments, the docetaxel and the human serum albumin in the composition are in a ratio by weight from about 1:90 to about 1:120. In some embodiments, the docetaxel and the human serum albumin in the composition are in a ratio by weight from about 1:100 to about 1:120. In some embodiments, the docetaxel and the human serum albumin in the composition are in a ratio by weight from about 1:90 to about 1:110. In some embodiments, the docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:170, about 1:180, about 1:190, about 1:200, about 1:210, about 1:220, or about 1:250. In some embodiments, the docetaxel and the human serum albumin in the composition are in a ratio by weight of about 1:90. In some embodiments, the docetaxel and the human serum albumin in the composition are in a ratio by weight of about 1:95. In some embodiments, the docetaxel and the human serum albumin in the composition are in a ratio by weight of about 1:100. In some embodiments, the docetaxel and the human serum albumin in the composition are in a ratio by weight of about 1:105. In some embodiments, the docetaxel and the human serum albumin in the composition are in a ratio by weight of about 1:110. In some embodiments, the docetaxel and the human serum albumin in the composition are in a ratio by weight of about 1:115. In some embodiments, the docetaxel and the human serum albumin in the composition are in a ratio by weight of about 1:120. In some embodiments, the docetaxel and the human serum albumin in the composition are in a ratio by weight of about 1:125. In some embodiments, the docetaxel and the human serum albumin in the composition are in a ratio by weight of about 1:130. In some embodiments, the docetaxel and the human serum albumin in the composition are in a ratio by weight of about 1:135. In some embodiments, the docetaxel and the human serum albumin in the composition are in a ratio by weight of about 1:140.

In some embodiments, the ratio of the amount of sodium caprylate to the amount of the proteins in the composition is from about 0.100 mmol to about 0.200 mmol of sodium caprylate per one gram of the proteins. In some embodiments, the ratio of the amount of sodium caprylate to the amount of the proteins in the composition is from about 0.120 mmol to about 0.190 mmol of sodium caprylate per one gram of the proteins. In some embodiments, the ratio of the amount of sodium caprylate to the amount of the proteins in the composition is from about 0.140 mmol to about 0.180 mmol of sodium caprylate per one gram of the proteins. In some embodiments, the ratio of the amount of sodium caprylate to the amount of the proteins in the composition is from about 0.145 mmol to about 0.175 mmol of sodium caprylate per one gram of the proteins. In some embodiments, the ratio of the amount of sodium caprylate to the amount of the proteins in the composition is from about 0.150 mmol to about 0.170 mmol of sodium caprylate per one gram of the proteins. In some embodiments, the ratio of the amount of sodium caprylate to the amount of the proteins in the composition is about 0.160 mmol of sodium caprylate per one gram of the proteins. In some embodiments, the ratio of the amount of sodium caprylate to the amount of the proteins in the composition is about 0.155 mmol of sodium caprylate per one gram of the proteins. In some embodiments, the ratio of the amount of sodium caprylate to the amount of the proteins in the composition is about 0.165 mmol of sodium caprylate per one gram of the proteins.

In some embodiments, provided herein is a composition comprising docetaxel, proteins comprising a human serum albumin, and sodium caprylate, wherein the docetaxel and the human serum albumin in the composition have a ratio by weight from about 1:70 to about 1:300, wherein the ratio of the amount of sodium caprylate to the amount of the proteins in the composition is from about 0.100 mmol to about 0.200 mmol of sodium caprylate for each one gram of the proteins, and wherein the composition does not contain sodium N-acetyl tryptophanate. In some embodiments, provided herein is a composition comprising docetaxel, proteins comprising a human serum albumin, and sodium caprylate, wherein the docetaxel and the human serum albumin in the composition have a ratio by weight from about 1:70 to about 1:300, wherein the ratio of the amount of sodium caprylate to the amount of the proteins in the composition is from about 0.140 mmol to about 0.180 mmol of sodium caprylate for each one gram of the proteins, and wherein the composition does not contain sodium N-acetyl tryptophanate.

In some embodiments, provided herein is a composition comprising docetaxel, proteins comprising a human serum albumin, and sodium caprylate, wherein the docetaxel and the human serum albumin in the composition have a ratio by weight from about 1:80 to about 1:200, wherein the ratio of the amount of sodium caprylate to the amount of the proteins in the composition is from about 0.100 mmol to about 0.200 mmol of sodium caprylate for each one gram of the proteins, and wherein the composition does not contain sodium N-acetyl tryptophanate. In some embodiments, provided herein is a composition comprising docetaxel, proteins comprising a human serum albumin, and sodium caprylate, wherein the docetaxel and the human serum albumin in the composition have a ratio by weight from about 1:80 to about 1:200, wherein the ratio of the amount of sodium caprylate to the amount of the proteins in the composition is from about 0.140 mmol to about 0.180 mmol of sodium caprylate for each one gram of the proteins, and wherein the composition does not contain sodium N-acetyl tryptophanate.

In some embodiments, provided herein is a composition comprising docetaxel, proteins comprising a human serum albumin, and sodium caprylate, wherein the docetaxel and the human serum albumin in the composition have a ratio by weight from about 1:90 to about 1:150, wherein the ratio of the amount of sodium caprylate to the amount of the proteins in the composition is from about 0.100 mmol to about 0.200 mmol of sodium caprylate for each one gram of the proteins, and wherein the composition does not contain sodium N-acetyl tryptophanate. In some embodiments, provided herein is a composition comprising docetaxel, proteins comprising a human serum albumin, and sodium caprylate, wherein the docetaxel and the human serum albumin in the composition have a ratio by weight from about 1:90 to about 1:150, wherein the ratio of the amount of sodium caprylate to the amount of the proteins in the composition is from about 0.140 mmol to about 0.180 mmol of sodium caprylate for each one gram of the proteins, and wherein the composition does not contain sodium N-acetyl tryptophanate.

In some embodiments, provided herein is a composition comprising docetaxel, proteins comprising a human serum albumin, and sodium caprylate, wherein the docetaxel and the human serum albumin in the composition have a ratio by weight from about 1:90 to about 1:130, wherein the ratio of the amount of sodium caprylate to the amount of the proteins in the composition is from about 0.100 mmol to about 0.200 mmol of sodium caprylate for each one gram of the proteins, and wherein the composition does not contain sodium N-acetyl tryptophanate. In some embodiments, provided herein is a composition comprising docetaxel, proteins comprising a human serum albumin, and sodium caprylate, wherein the docetaxel and the human serum albumin in the composition have a ratio by weight from about 1:90 to about 1:130, wherein the ratio of the amount of sodium caprylate to the amount of the proteins in the composition is from about 0.140 mmol to about 0.180 mmol of sodium caprylate for each one gram of the proteins, and wherein the composition does not contain sodium N-acetyl tryptophanate.

In some embodiments, provided herein is a composition comprising docetaxel, proteins comprising a human serum albumin, and sodium caprylate, wherein the docetaxel and the human serum albumin in the composition have a ratio by weight of about 1:80, about 1:85, about 1:90, about 1:95, about 1:100, about 1:105, about 1:110, about 1:115, about 1:120, about 1:125, about 1:130, about 1:135, about 1:140, or about 1:145, wherein the ratio of the amount of sodium caprylate to the amount of the proteins in the composition is from about 0.100 mmol to about 0.200 mmol of sodium caprylate for each one gram of the proteins, and wherein the composition does not contain sodium N-acetyl tryptophanate. In some embodiments, provided herein is a composition comprising docetaxel, proteins comprising a human serum albumin, and sodium caprylate, wherein the docetaxel and the human serum albumin in the composition have a ratio by weight of about 1:80, about 1:85, about 1:90, about 1:95, about 1:100, about 1:105, about 1:110, about 1:115, about 1:120, about 1:125, about 1:130, about 1:135, about 1:140, or about 1:145, wherein the ratio of the amount of sodium caprylate to the amount of the proteins in the composition is from about 0.140 mmol to about 0.180 mmol of sodium caprylate for each one gram of the proteins, and wherein the composition does not contain sodium N-acetyl tryptophanate.

In some embodiments, provided herein is a composition comprising docetaxel, proteins comprising a human serum albumin, and sodium caprylate, wherein the docetaxel and the human serum albumin in the composition have a ratio by weight of about 1:120, wherein the ratio of the amount of sodium caprylate to the amount of the proteins in the composition is from about 0.100 mmol to about 0.200 mmol of sodium caprylate for each one gram of the proteins, and wherein the composition does not contain sodium N-acetyl tryptophanate. In some embodiments, provided herein is a composition comprising docetaxel, proteins comprising a human serum albumin, and sodium caprylate, wherein the docetaxel and the human serum albumin in the composition have a ratio by weight of about 1:120, wherein the ratio of the amount of sodium caprylate to the amount of the proteins in the composition is from about 0.140 mmol to about 0.180 mmol of sodium caprylate for each one gram of the proteins, and wherein the composition does not contain sodium N-acetyl tryptophanate.

In some embodiments, provided herein is a composition comprising docetaxel, proteins comprising a human serum albumin, and sodium caprylate, wherein the docetaxel and the human serum albumin in the composition have a ratio by weight of about 1:100, wherein the ratio of the amount of sodium caprylate to the amount of the proteins in the composition is from about 0.100 mmol to about 0.200 mmol of sodium caprylate for each one gram of the proteins, and wherein the composition does not contain sodium N-acetyl tryptophanate. In some embodiments, provided herein is a composition comprising docetaxel, proteins comprising a human serum albumin, and sodium caprylate, wherein the docetaxel and the human serum albumin in the composition have a ratio by weight of about 1:100, wherein the ratio of the amount of sodium caprylate to the amount of the proteins in the composition is from about 0.140 mmol to about 0.180 mmol of sodium caprylate for each one gram of the proteins, and wherein the composition does not contain sodium N-acetyl tryptophanate.

As used herein, the term “human serum albumin” refers to native and recombinant human serum albumin. Native human serum albumin and other plasma proteins can be precipitated from human plasma by varying the pH and adding ethanol, in what is known as the Cohn fractionation process (see, e.g. Cohn E J et al., J. Am. Chem. Soc. 1946; 68:459-475). By controlling the pH and ethanol content, semi-purified fractions of plasma proteins can be produced. One of the last proteins to precipitate in the Cohn process is native human serum albumin. After precipitation, a wet paste of crude native human serum albumin is obtained. Subsequent bioprocessing steps (purification, filtration, pasteurization, etc.) can be used to produce a purified, stabilized form of native human serum albumin for commercial use (Lin J J et al., Pharmaceutical Research 2000; 17:391-6). Recombinant human serum albumin is a highly purified animal-, virus-, and prion-free product as alternative to native human serum albumin, to which it is structurally equivalent (Bosse D et al., J. Clin. Pharmacol. 2005; 45:57-67). Recombinant human serum albumin has been produced by various hosts, both prokaryotic and eukaryotic (Chen Z et al., Biochimica et Biophysica Acta 2013; 1830:5515-5525).

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see. e.g., Tullis, JAMA, 237, 355-360, 460-463, (1977) and Houser et al., Surgery. Gynecology and Obstetrics, 150, 811-816 (1980)) and in conjunction with exchange transfusion in the treatment of neonatal hyperbilirubinemia (see. e.g., Finlayson, Seminars in Thrombosis and Hemostasis, 6, 85-120, (1980)).

Human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (1981), Vorum, Dan. Med. Bull., 46, 379-99 (1999), Kragh-Hansen, Dan. Med Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (1992), and Carter et al., Adv. Protein. Chem., 45, 153-203 (1994)).

In some embodiments, the human serum albumin is a native human serum albumin. In some embodiments, the human serum albumin is a native human serum albumin obtained from pools of human plasma. In some embodiments, the human serum albumin is a native human serum albumin obtained from pools of human plasma by low temperature controlled fractionation according to the Cohn process.

As used herein, the term “proteins” refers to plasma proteins from human plasma. Plasma proteins, also termed blood proteins or serum proteins, are proteins present in human blood plasma. Human serum albumin accounts for 55% of blood proteins. Globulins make up 38% of blood proteins and transport ions, hormones, and lipids assisting in immune function. Fibrinogen comprises 7% of blood proteins; conversion of fibrinogen to insoluble fibrin is essential for blood clotting. The remainder of the plasma proteins (1%) are regulatory proteins, such as enzymes, proenzymes, and hormones.

In vitro studies showed that docetaxel is about 94% protein bound, mainly to al-acid glycoprotein, albumin, and lipoproteins. In three cancer patients, the in vitro binding to plasma proteins was found to be approximately 97%. See Docetaxel Prescribing Information.

As used herein the term “docetaxel” refers to a compound that has the CAS No. 114977-28-5 and the following chemical structure:

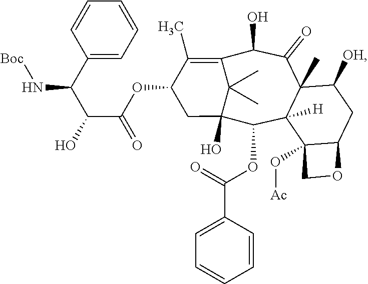

or a pharmaceutically acceptable salt thereof.

Docetaxel is a white to almost-white powder. It is highly lipophilic and practically insoluble in water.

Further, docetaxel is a microtubule inhibitor indicated for breast cancer, non-small cell lung cancer, hormone refractory prostate cancer, gastric adenocarcinoma, and squamous cell carcinoma of the head and neck cancer.

In some embodiments, the term “docetaxel” encompasses a pharmaceutically acceptable salt of docetaxel.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxy benzoate, methoxy benzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), napthalene-2-sulfonate, ethanedisulfonate, hydrogen bisulfide, bitartrate, gluconate, glucuronate, para-bromophenylsulfonate, carbonate, pyrosulfate, sulfite, bisulfite, monohydrogen phosphate, dihydrogen phosphate, metaphosphate, pyrophosphate, chloride, bromide, iodide, decanoate, caprylate, caprate, propiolate, suberate, sebacate, butyne-1,4-dioate, hexyne-1,6-dioate, terephthalate, sulfonate, xylenesulfonate, phenylpropionate, phenylbutyrate, β-hydroxy butyrate, glycolate, propanesulfonate, naphthalene-1-sulfonate, naphthalene-2-sulfonate and 2,5-dihydroxy benzoate. Suitable bases include pharmaceutically acceptable inorganic bases and pharmaceutically acceptable organic bases. Representative pharmaceutically acceptable base addition salts include hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the docetaxel can be a docetaxel with 1, 2, or 3 equivalents of the water solvate. In some embodiments, the docetaxel can be a docetaxel with three equivalents of the water solvate. In some embodiments, docetaxel is the docetaxel trihydrate. In some embodiments, docetaxel is the docetaxel monohydrate. In some embodiments, docetaxel is the docetaxel anhydrous. In some embodiments, the docetaxel can be a docetaxel with one equivalent of the acetone solvate. In some embodiments, the docetaxel can be any one of docetaxel solvates disclosed, for example, in WO2010091650 or US2012007167, the disclosures of which are incorporated herein by reference in its entirety.

In some embodiments, docetaxel is crystalline. In some embodiments, docetaxel is any one of the crystalline forms disclosed, for example, in WO2012115402, U.S. Pat. No. 8,410,294, US20100197944, US20100099897, U.S. Pat. No. 8,357,811, US20100160653, or US20070142457, the disclosures of which are incorporated herein by reference in their entirety.

In some embodiments, docetaxel in amorphous. In some embodiments, Docetaxel is any one of the amorphous forms disclosed, for example, in WO2008102374, the disclosure of which is incorporated herein by reference in its entirety.

As used herein, the term “sodium caprylate” refers to a compound that has the following chemical structure:

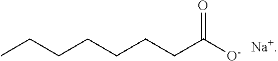

Sodium caprylate can also be called as sodium n-octanoate or sodium octanoate. The CAS Registry No. for sodium caprylate is 1984-06-1.

As used herein, the term “sodium N-acetyltryptophanate” refers to a compound that has the following chemical structure:

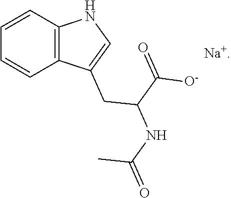 [SMILES: CC(=O)NC(Cc1c[nH]c2ccccc12)C(=O)[O-]]

Sodium N-acetyltryptophanate can also be called as sodium N-acetyl-DL-tryptophanate or sodium acetyltryptophanate. The CAS Registry No. for sodium acetyltryptophanate is 62307-74-8.

Sodium caprylate and sodium N-acetyltryptophanate are the stabilizers used in the intravenous Human Albumin (human serum albumin) solution for infusion (e.g. Human Albumin; prepared as a 5%, 20%, or 25% protein solution). These products are pasteurized at 60° C. for at least 10 hours for the inactivation of hepatitis, HIV, or other viruses. Sodium caprylate and sodium N-acetyltryptophanate are the stabilizers used in pasteurization process.

The intravenous Human Albumin (human serum albumin) for infusion (e.g. Human Albumin USP; prepared as a 5%, 20%, or 25% protein solution) is a sterile aqueous solution of albumin obtained from large pools of adult human venous plasma by low temperature controlled fractionation according to the Cohn process. Human Albumin solution for infusion is a clear, slightly viscous liquid; it is almost colorless or slightly yellow or green. Human Albumin solution for infusion contains protein, of which at least 96% is human albumin. Human Albumin 5% solution for infusion is mildly hypooncotic to normal human plasma and contains, in each 100 mL, 5 grams of protein, of which at least 96% is human albumin. Human Albumin 25% solution for infusion is hyperoncotic to normal human plasma and contains, in each 100 mL, 25 grams of protein, of which at least 96% is human albumin. The solution contains no preservative.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use.

In some embodiments, the composition is a solid formulation. For example, the solid formulation can be produced in a uniform manner by lyophilization. A skilled artisan would recognize other methods, such as rotary evaporation, that can also produce solid formulations. In some embodiments, the pH of the solid formulation is neutral (e.g., pH of the composition is from about 5 to about 8, from about 5.5 to about 7.5, or from about 6 to about 7.5, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the composition is an aqueous formulation. In some embodiments, the aqueous formulation is substantially free of solvent other than water. In some embodiments, the aqueous formulation is free of solvent other than water. In some embodiments, the pH of the aqueous formulation (e.g., clear aqueous solution) is neutral (e.g., pH of the composition is from about 5 to about 8, from about 5.5 to about 7.5, from about 6 to about 7.5, from about 6.5 to about 7.5, or from about 6 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent. In some embodiments, the aqueous solution contains less than 0.01%, by weight, of any non-water solvent.

In some embodiments, the aqueous formulation can be substantially free of a surfactant selected from the group consisting of CREMOPHOR® surfactants and Polysorbate 80. In some embodiments, the aqueous formulation can be free of a surfactant selected from the group consisting of CREMOPHOR R; surfactants and Polysorbate 80.

As used herein, the term “substantially free of surfactant” refers to a formulation containing less than 0.0005%, less than 0.0003%, or less than 0.0001% of a surfactant selected from the group consisting of CREMOPHOR® surfactants and Polysorbate 80.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

As used herein, the term “clear aqueous solution” refers to an aqueous solution containing Docetaxel and HSA that is transparent and optically clear upon visual observation and essentially free of visible particles or precipitation of undissolved docetaxel.

The term “essentially free of visible particles or precipitation of undissolved docetaxel” can be assessed as follows: after a clear aqueous solution is filtered with a 0.22 micron filter, the amount of docetaxel in the filtered aqueous solution is at least 95% of the total amount of docetaxel in the aqueous solution before filtration. The total amount of docetaxel in the aqueous solution before filtration includes the particles or precipitation of undissolved docetaxel in the aqueous solution or with the aqueous solution. The amount of the docetaxel in an aqueous solution can be measured by the methods using HPLC. The methods of measuring the amount of the docetaxel in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

As used herein, the term “micron” refers to a unit of measure of one one-thousandth of a millimeter. In some embodiments, the term “micron” refers to a micrometer.

As used herein, the term “aqueous solution” refers to a solution, wherein at least one solvent is water and the weight % of water in the mixture of solvents is at least 50%, at least 60%, at least 70% or at least 90%. In some embodiments, aqueous solution is a solution in which water is the only solvent.

As used herein, the term “aqueous solvent” refers to a liquid comprising at least 50%, at least 60%, at least 70%, at least 90% or at least 95% water. In some embodiments, aqueous solvent is water.

In some embodiments, the aqueous formulation is a clear aqueous solution reconstituted from the solid formulation (e.g. the sterile lyophilized powder) in water. In some embodiments, the aqueous formulation is a clear aqueous solution reconstituted from the solid formulation (e.g. the sterile lyophilized powder) in 0.9% saline solution. In some embodiments, the aqueous formulation is a clear aqueous solution reconstituted from the solid formulation (e.g. the sterile lyophilized powder) in 5% dextrose water solution.

In some embodiments, the aqueous formulation is a clear aqueous solution reconstituted from the solid formulation (e.g. the sterile lyophilized powder) in an aqueous solvent, wherein the aqueous formulation has pH value from about 5 to about 8. In some embodiments, the aqueous formulation is a clear aqueous solution reconstituted from the solid formulation (e.g. the sterile lyophilized powder) in water, wherein the aqueous formulation has pH value from about 5 to about 8. In some embodiments, the aqueous formulation is a clear aqueous solution reconstituted from the solid formulation (e.g. the sterile lyophilized powder) in 0.9% saline solution, wherein the aqueous formulation has pH value from about 5 to about 8. In some embodiments, the aqueous formulation is a clear aqueous solution reconstituted from the solid formulation (e.g. the sterile lyophilized powder) in 5% dextrose water solution, wherein the aqueous formulation has pH value from about 5 to about 8.

In some embodiments, the aqueous formulation is a clear aqueous solution reconstituted from the solid formulation (e.g. the sterile lyophilized powder) in an aqueous solvent, wherein the aqueous formulation has pH value from about 6 to about 7.5. In some embodiments, the aqueous formulation is a clear aqueous solution reconstituted from the solid formulation (e.g. the sterile lyophilized powder) in water, wherein the aqueous formulation has pH value from about 6 to about 7.5. In some embodiments, the aqueous formulation is a clear aqueous solution reconstituted from the solid formulation (e.g. the sterile lyophilized powder) in 0.9% saline solution, wherein the aqueous formulation has pH value from about 6 to about 7.5. In some embodiments, the aqueous formulation is a clear aqueous solution reconstituted from the solid formulation (e.g. the sterile lyophilized powder) in 5% dextrose water solution, wherein the aqueous formulation has pH value from about 6 to about 7.5.

In some embodiments, the aqueous formulation is a clear aqueous solution reconstituted from the solid formulation (e.g. the sterile lyophilized powder) in an aqueous solvent, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8. In some embodiments, the aqueous formulation is a clear aqueous solution reconstituted from the solid formulation (e.g. the sterile lyophilized powder) in water, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8. In some embodiments, the aqueous formulation is a clear aqueous solution reconstituted from the solid formulation (e.g. the sterile lyophilized powder) in 0.9% saline solution, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8. In some embodiments, the aqueous formulation is a clear aqueous solution reconstituted from the solid formulation (e.g., the sterile lyophilized powder) in 5% dextrose solution in water, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8.

In some aspects of the aforementioned embodiments, the concentration of the reconstituted solid (e.g., the sterile lyophilized powder comprising docetaxel, HSA, and sodium caprylate) in the aqueous formulation is about 25 mg, about 30 mg, about 35 mg, about 40 mg, about 45 mg, about 50 mg, about 55 mg, about 60 mg, about 65 mg, about 70 mg, about 75 mg, about 80 mg, about 100 mg, about 150 mg, or 200 mg per 1 ml of the aqueous solvent. In some aspects of the aforementioned embodiments, the concentration of the reconstituted solid (e.g., the sterile lyophilized powder comprising docetaxel, HSA, and sodium caprylate) in the aqueous formulation is from about 10 mg per 1 ml to about 250 mg per 1 ml of the aqueous solvent. In some aspects of the aforementioned embodiments, the concentration of the reconstituted solid (e.g., the sterile lyophilized powder comprising docetaxel, HSA, and sodium caprylate) in the aqueous formulation is from about 25 mg per 1 ml to about 150 mg per 1 ml of the aqueous solvent. In some aspects of the aforementioned embodiments, the concentration of the reconstituted solid (e.g., the sterile lyophilized powder comprising docetaxel, HSA, and sodium caprylate) in the aqueous formulation is from about 25 mg per 1 ml to about 100 mg per 1 ml of the aqueous solvent. In some aspects of the aforementioned embodiments, the concentration of the reconstituted solid (e.g., the sterile lyophilized powder comprising docetaxel, HSA, and sodium caprylate) in the aqueous formulation is from about 25 mg per 1 ml to about 80 mg per 1 ml of the aqueous solvent. In some aspects of the aforementioned embodiments, the concentration of the reconstituted solid (e.g., the sterile lyophilized powder comprising docetaxel, HSA, and sodium caprylate) in the aqueous formulation is from about 30 mg per 1 ml to about 70 mg per 1 ml of the aqueous solvent. In some aspects of the aforementioned embodiments, the concentration of the reconstituted solid (e.g., the sterile lyophilized powder comprising docetaxel, HSA, and sodium caprylate) in the aqueous formulation is from about 30 mg per 1 ml to about 50 mg per 1 ml of the aqueous solvent.

In some embodiments, the aqueous formulation has pH value from about 5 to about 8. In some embodiments, the aqueous formulation has pH value from about 5.5 to about 7.8. In some embodiments, the aqueous formulation has pH value from about 6 to about 7.5. In some embodiments, the aqueous formulation has pH value from about 6.5 to about 7.5. In some embodiments, the aqueous formulation has pH value from about 6 to about 6.5. In some embodiments, the aqueous formulation has pH value from about 6.5 to about 7. In some embodiments, the aqueous formulation has pH value from about 7 to about 7.5. In some embodiments, the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, the aqueous formulation is a clear aqueous solution, wherein the aqueous formulation has pH value from about 5 to about 8, and wherein the aqueous formulation is substantially free of solvent other than water. In some embodiments, the aqueous formulation is a clear aqueous solution, wherein the aqueous formulation has pH value from about 5 to about 8, and wherein the aqueous formulation is free of solvent other than water. In some embodiments, the aqueous formulation is a clear aqueous solution, wherein the aqueous formulation has pH value from about 6 to about 7.5, and wherein the aqueous formulation is substantially free of solvent other than water. In some embodiments, the aqueous formulation is a clear aqueous solution, wherein the aqueous formulation has pH value from about 6 to about 7.5, and wherein the aqueous formulation is free of solvent other than water.

In some embodiments, after a clear aqueous solution is filtered by a 0.22 micron filter, the amount of docetaxel in the filtered aqueous solution is at least 96% of the total amount of docetaxel in the aqueous solution before filtration. In some embodiments, after a clear aqueous solution is filtered by a 0.22 micron filter, the amount of docetaxel in the filtered aqueous solution is at least 97% of the total amount of docetaxel in the aqueous solution before filtration. In some embodiments, after a clear aqueous solution is filtered by a 0.22 micron filter, the amount of docetaxel in the filtered aqueous solution is at least 98% of the total amount of docetaxel in the aqueous solution before filtration. In some embodiments, after a clear aqueous solution is filtered by a 0.22 micron filter, the amount of docetaxel in the filtered aqueous solution is at least 99% of the total amount of docetaxel in the aqueous solution before filtration. In some embodiments, after a clear aqueous solution is filtered by a 0.22 micron filter, the amount of docetaxel in the filtered aqueous solution is at least 99.5% of the total amount of docetaxel in the aqueous solution before filtration. In some embodiments, the aqueous formulation is substantially free of solvent other than water. In some embodiments, the aqueous formulation is free of solvent other than water.

In some embodiments, after the aqueous formulation (e.g. a clear aqueous solution) is filtered by a 0.22 micron filter, the amount of docetaxel in the filtered aqueous solution is at least 96%, 97%, 98%, 99%, or 99.5% of the total amount of docetaxel in the aqueous solution before filtration, wherein the clear aqueous solution has pH value from about 5 to about 8, and wherein the clear aqueous solution is substantially free of solvent other than water. In some embodiments, after the aqueous formulation (e.g. a clear aqueous solution) is filtered by a 0.22 micron filter, the amount of docetaxel in the filtered aqueous solution is at least 96%, 97%, 98%, 99%, or 99.5% of the total amount of docetaxel in the aqueous solution before filtration, wherein the clear aqueous solution has pH value from about 6 to about 7.5, and wherein the clear aqueous solution is substantially free of solvent other than water.

In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours. In some embodiments, the aqueous formulation is a clear aqueous solution for at least 1 hour, 2 hours, 3 hours, 4 hours, 5 hours, 6 hours, 7 hours, 8 hours, 10) hours, 12 hours, or 24 hours at a temperature from about 20° C. to about 25° C. In some embodiments, the aqueous formulation is a clear aqueous solution for at least 2 hours at a temperature from about 20° C. to about 25° C. In some embodiments, the aqueous formulation is a clear aqueous solution for at least 3 hours at a temperature from about 20° C. to about 25° C. In some embodiments, the aqueous formulation is a clear aqueous solution for at least 4 hours at a temperature from about 20° C. to about 25° C. In some embodiments, the aqueous formulation is a clear aqueous solution for at least 5 hours at a temperature from about 20° C. to about 25° C. In some embodiments, the aqueous formulation is a clear aqueous solution for at least 6 hours at a temperature from about 20° C. to about 25° C. In some embodiments, the aqueous formulation is a clear aqueous solution for at least 8 hours at a temperature from about 20° C. to about 25° C. In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours at a temperature from about 1° C. to about 10° C. In some embodiments, the aqueous formulation is a clear aqueous solution for at least 12 hours at a temperature from about 1° C. to about 10° C. In some embodiments, the aqueous formulation is a clear aqueous solution for at least 24 hours at a temperature from about 1° C. to about 10° C. In some embodiments, the aqueous formulation is substantially free of solvent other than water. In some embodiments, the aqueous formulation is free of solvent other than water.

In some embodiments, the aqueous formulation is a clear aqueous solution for at least 2 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 3 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 4 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 5 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 6 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 8 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 12 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 24 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is substantially free of solvent other than water. In some embodiments, the aqueous formulation is free of solvent other than water.

In some embodiments, the concentration of docetaxel in the aqueous formulation is from about 0.1 mg per 1 ml to about 2 mg per 1 ml of the aqueous solvent. In some embodiments, the concentration of docetaxel in the aqueous formulation is from about 0.1 mg per 1 ml to about 1 mg per 1 ml of the aqueous solvent. In some embodiments, the concentration of docetaxel in the aqueous formulation is from about 0.2 mg per 1 ml to about 0.9 mg per 1 ml of the aqueous solvent. In some embodiments, the concentration of docetaxel in the aqueous formulation is from about 0.25 mg per 1 ml to about 0.8 mg per 1 ml of the aqueous solvent. In some embodiments, the concentration of docetaxel in the aqueous formulation is from about 0.3 mg per 1 ml to about 0.75 mg per 1 ml of the aqueous solvent. In some embodiments, the concentration of docetaxel in the aqueous formulation is from about 0.3 mg per 1 ml to about 0.65 mg per 1 ml of the aqueous solvent. In some embodiments, the concentration of docetaxel in the aqueous formulation is from about 0.33 mg per 1 ml to about 0.5 mg per 1 ml of the aqueous solvent. In some embodiments, the concentration of docetaxel in the aqueous formulation is from about 0.2 mg per 1 ml to about 0.3 mg per 1 ml of the aqueous solvent. In some embodiments, the concentration of docetaxel in the aqueous formulation is from about 0.3 mg per 1 ml to about 0.4 mg per 1 ml of the aqueous solvent. In some embodiments, the concentration of docetaxel in the aqueous formulation is from about 0.4 mg per 1 ml to about 0.5 mg per 1 ml of the aqueous solvent. In some embodiments, the concentration of docetaxel in the aqueous formulation is about 0.25 mg per 1 ml of the aqueous solvent. In some embodiments, the concentration of docetaxel in the aqueous formulation is about 0.3 mg per 1 ml of the aqueous solvent. In some embodiments, the concentration of docetaxel in the aqueous formulation is about 0.33 mg per 1 ml of the aqueous solvent. In some embodiments, the concentration of docetaxel in the aqueous formulation is about 0.4 mg per 1 ml of the aqueous solvent. In some embodiments, the concentration of docetaxel in the aqueous formulation is about 0.45 mg per 1 ml of the aqueous solvent. In some embodiments, the concentration of docetaxel in the aqueous formulation is about 0.5 mg per 1 ml of the aqueous solvent. In some embodiments, the aqueous formulation is substantially free of solvent other than water. In some embodiments, the aqueous formulation is free of solvent other than water.

In some embodiments, the aqueous formulation is a clear aqueous solution, wherein the concentration of docetaxel in the aqueous formulation is from about 0.1 mg per 1 ml to about 2 mg per 1 ml of the aqueous solvent. In some embodiments, the aqueous formulation is a clear aqueous solution, wherein the concentration of docetaxel in the aqueous formulation is from about 0.1 mg per 1 ml to about 1 mg per 1 ml of the aqueous solvent. In some embodiments, the aqueous formulation is a clear aqueous solution, wherein the concentration of docetaxel in the aqueous formulation is from about 0.2 mg per 1 ml to about 0.8 mg per 1 ml of the aqueous solvent. In some embodiments, the aqueous formulation is a clear aqueous solution, wherein the concentration of docetaxel in the aqueous formulation is from about 0.3 mg per 1 ml to about 0.75 mg per 1 ml of the aqueous solvent. In some embodiments, the aqueous formulation is a clear aqueous solution, wherein the concentration of docetaxel in the aqueous formulation is from about 0.3 mg per 1 ml to about 0.65 mg per 1 ml of the aqueous solvent. In some embodiments, the aqueous formulation is a clear aqueous solution, wherein the concentration of docetaxel in the aqueous formulation is from about 0.33 mg per 1 ml to about 0.5 mg per 1 ml of the aqueous solvent. In some embodiments, the aqueous formulation is a clear aqueous solution, wherein the concentration of docetaxel in the aqueous formulation is from about 0.25 mg per 1 ml to about 0.35 mg per 1 ml of the aqueous solvent. In some embodiments, the aqueous formulation is a clear aqueous solution, wherein the concentration of docetaxel in the aqueous formulation is from about 0.35 mg per 1 ml to about 0.5 mg per 1 ml of the aqueous solvent. In some embodiments, the aqueous formulation is a clear aqueous solution, wherein the concentration of docetaxel in the aqueous formulation is about 0.3 mg per 1 ml of the aqueous solvent. In some embodiments, the aqueous formulation is a clear aqueous solution, wherein the concentration of docetaxel in the aqueous formulation is about 0.33 mg per 1 ml of the aqueous solvent. In some embodiments, the aqueous formulation is a clear aqueous solution, wherein the concentration of docetaxel in the aqueous formulation is about 0.4 mg per 1 ml of the aqueous solvent. In some embodiments, the aqueous formulation is a clear aqueous solution, wherein the concentration of docetaxel in the aqueous formulation is about 0.5 mg per 1 ml of the aqueous solvent. In some embodiments, the aqueous formulation is substantially free of solvent other than water. In some embodiments, the aqueous formulation is free of solvent other than water.

In some embodiments, the aqueous formulation is a clear aqueous solution, wherein the concentration of docetaxel in the aqueous formulation is from about 0.1 mg per 1 ml to about 1 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.2 mg per 1 ml to about 0.8 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.3 mg per 1 ml to about 0.75 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.3 mg per 1 ml to about 0.65 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.33 mg per 1 ml to about 0.5 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.25 mg per 1 ml to about 0.35 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.25 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.3 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.33 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.4 mg per 1 ml of the aqueous solvent, and wherein the aqueous formulation is substantially free of solvent other than water.

In some embodiments, provided herein is a composition comprising docetaxel, proteins comprising a human serum albumin, and sodium caprylate, wherein the docetaxel and the human serum albumin in the composition have a ratio by weight from about 1:80 to about 1:200, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.1 mg per 1 ml to about 1 mg per 1 ml of the aqueous solvent, and wherein the aqueous formulation is substantially free of solvent other than water. In some embodiments, provided herein is a composition comprising docetaxel, proteins comprising human serum albumin, and sodium caprylate, wherein the docetaxel and the human serum albumin in the composition have a ratio by weight from about 1:90 to about 1:150, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.1 mg per 1 ml to about 1 mg per 1 ml of the aqueous solvent, and wherein the aqueous formulation is substantially free of solvent other than water. In some embodiments, provided herein is a composition comprising docetaxel, proteins comprising a human serum albumin and sodium caprylate, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.1 mg per 1 ml to about 1 mg per 1 ml of the aqueous solvent, and wherein the aqueous formulation is substantially free of solvent other than water.

In some embodiments, provided herein is a composition comprising docetaxel, proteins comprising a human serum albumin, and sodium caprylate, wherein the docetaxel and the human serum albumin in the composition have a ratio by weight from about 1:80 to about 1:200, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.25 mg per 1 ml to about 0.65 mg per 1 ml of the aqueous solvent, and wherein the aqueous formulation is substantially free of solvent other than water. In some embodiments, provided herein is a composition comprising docetaxel, proteins comprising a human serum albumin, and sodium caprylate, wherein the docetaxel and the human serum albumin in the composition have a ratio by weight from about 1:90 to about 1:150, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.25 mg per 1 ml to about 0.65 mg per 1 ml of the aqueous solvent, and wherein the aqueous formulation is substantially free of solvent other than water. In some embodiments, provided herein is a composition comprising docetaxel, proteins comprising a human serum albumin and sodium caprylate, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.25 mg per 1 ml to about 0.65 mg per 1 ml of the aqueous solvent, and wherein the aqueous formulation is substantially free of solvent other than water.

In some embodiments, provided herein is a composition comprising docetaxel, proteins comprising a human serum albumin, and sodium caprylate, wherein the docetaxel and the human serum albumin in the composition have a ratio by weight from about 1:80 to about 1:200, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is about 0.25 mg per 1 ml, about 0.3 mg per 1 ml, about 0.33 mg per 1 ml, about 0.4 mg per 1 ml, or about 0.5 mg per 1 ml of the aqueous solvent, and wherein the aqueous formulation is substantially free of solvent other than water. In some embodiments, provided herein is a composition comprising docetaxel, proteins comprising a human serum albumin and sodium caprylate, wherein the docetaxel and the human serum albumin in the composition have a ratio by weight from about 1:90 to about 1:150, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is about 0.25 mg per 1 ml, about 0.3 mg per 1 ml, about 0.33 mg per 1 ml, about 0.4 mg per 1 ml, or about 0.5 mg per 1 ml of the aqueous solvent, and wherein the aqueous formulation is substantially free of solvent other than water. In some embodiments, provided herein is a composition comprising docetaxel, proteins comprising a human serum albumin, and sodium caprylate, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is about 0.25 mg per 1 ml, about 0.3 mg per 1 ml, about 0.33 mg per 1 ml, about 0.4 mg per 1 ml, or about 0.5 mg per 1 ml of the aqueous solvent, and wherein the aqueous formulation is substantially free of solvent other than water.

In some embodiments, provided herein is a composition comprising docetaxel, proteins comprising a human serum albumin, and sodium caprylate, wherein at least 96% of the proteins in the composition is human serum albumin, wherein the docetaxel and the human serum albumin in the composition have a ratio by weight from about 1:80 to about 1:200, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.3 mg per 1 ml to about 0.4 mg per 1 ml of the aqueous solvent, and wherein the aqueous formulation is substantially free of solvent other than water. In some embodiments, provided herein is a composition comprising docetaxel, proteins comprising a human serum albumin, and sodium caprylate, wherein at least 96% of the proteins in the composition is human serum albumin, wherein the docetaxel and the human serum albumin in the composition have a ratio by weight from about 1:90 to about 1:150, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.3 mg per 1 ml to about 0.4 mg per 1 ml of the aqueous solvent, and wherein the aqueous formulation is substantially free of solvent other than water. In some embodiments, provided herein is a composition comprising docetaxel, proteins comprising a human serum albumin, and sodium caprylate, wherein at least 96% of the proteins in the composition is human serum albumin, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, wherein the ratio of the amount of sodium caprylate to the amount of the proteins in the composition is from about 0.140 mmol to about 0.180 mmol of sodium caprylate for each one gram of the proteins, wherein the composition does not contain sodium N-acetyl tryptophanate, wherein the composition is an aqueous formulation, wherein the concentration of docetaxel in the aqueous formulation is from about 0.3 mg per 1 ml to about 0.4 mg per 1 ml of the aqueous solvent, and wherein the aqueous formulation is substantially free of solvent other than water.

Also, provided herein is a pharmaceutical composition comprising the composition comprising docetaxel, proteins comprising a human serum albumin, and sodium caprylate as described herein, and a pharmaceutically acceptable carrier.

In some embodiments, the pharmaceutical composition further comprises at least one anti-cancer drug (e.g., any one of the anti-cancer drugs as described herein).

As used herein, the term “pharmaceutically acceptable carrier” is meant any solution used to solubilize and deliver an agent to a subject. A desirable pharmaceutically acceptable carrier is saline or water. Other pharmaceutically acceptable carrier and their formulation are known to one skilled in the art and described, for example, in Remington's Pharmaceutical Sciences. (20th edition), ed. A. Gennaro, 2003, Lippincon Williams & Wilkins.

Pharmaceutically acceptable carriers that may be used in the pharmaceutical compositions of the present application include, but are not limited to, ion exchangers, alumina, aluminum stearate, lecithin, serum proteins (other than HSA), buffer substances such as phosphates, glycine, sorbic acid, potassium sorbate, salts or electrolytes, such as protamine sulfate, disodium hydrogen phosphate, potassium hydrogen phosphate, sodium chloride, zinc salts, colloidal silica, magnesium trisilicate, and cellulose-based substances.

In some embodiments, the pharmaceutical composition is free of a surfactant selected from the group consisting of CREMOPHOR® surfactants and Polysorbate 80. In some embodiments, the pharmaceutical composition is substantially free of a surfactant selected from the group consisting of CREMOPHOR® surfactants and Polysorbate 80.

Also, provided herein is a method of treating a proliferative disease comprising the step of administering to a subject in need thereof a therapeutically effective amount of a pharmaceutical composition comprising the composition comprising docetaxel, proteins comprising a human serum albumin, and sodium caprylate as described herein, and a pharmaceutically acceptable carrier.

As used herein, the terms “individual”, “patient”, or “subject” are used interchangeably and refer to any animal, including mammals, preferably mice, rats, other rodents, rabbits, dogs, cats, swine, cattle, sheep, horses, or primates, and most preferably humans.

As used herein, the term “proliferative disease” refers to a disease caused by excessive proliferation of cells and turnover of cellular matrix. Non-limiting examples of proliferative diseases include cancer, atherosclerosis, arthritis (e.g. rheumatoid arthritis), psoriasis, fibrosis (e.g. pulmonary fibrosis, idiopathic pulmonary fibrosis), scleroderma and cirrhosis (e.g. cirrhosis of the liver).

Also, provided herein is a method of treating a cancer, the method comprising the step of administering to a subject in need thereof of a therapeutically effective amount of a pharmaceutical composition comprising the composition comprising docetaxel, proteins comprising a human serum albumin, and sodium caprylate as described herein, and a pharmaceutically acceptable carrier.

In some embodiments, the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

In some embodiments, cancer is selected from sarcoma, angiosarcoma, fibrosarcoma, rhabdomyosarcoma, liposarcoma, myxoma, rhabdomyoma, fibroma, lipoma, teratoma, non-small cell lung cancer (NSCLC), bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

As used herein, an “effective amount,” “therapeutically effective amount,” or a “pharmaceutically-effective amount” in reference to the compounds or compositions of the instant invention refers to the amount sufficient to induce a desired biological, pharmacological, or therapeutic outcome in a subject. That result can be reduction, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In cancer treatment, the result will generally include the reduction, mitigation, limitation, and/or, delay of the deleterious physiological manifestations, growth or metastases of neoplasms.

In some embodiments, the cancer is a solid tumor cancer. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma. In some embodiments, the cancer is a breast cancer. In some embodiments, the cancer is a non-small cell lung cancer. In some embodiments, the cancer is a prostate cancer. In some embodiments, the cancer is a gastric cancer. In some embodiments, the cancer is a head and neck cancer. In some embodiments the cancer is an ovarian cancer. In some embodiments, the cancer is a pancreatic cancer. In some embodiments, the cancer is a Kaposi's sarcoma.

In some embodiments, the method of treating cancer (e.g. any one of cancers described herein) comprises the step of administering to a subject in need thereof of a therapeutically effective amount of a composition comprising docetaxel, proteins comprising a human serum albumin, and sodium caprylate as described herein, and a therapeutically effective amount of at least one inhibitor of the following kinases for the treatment of cancer: PIM, Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf.

In some embodiments, the method of treating cancer (e.g. any one of cancers described herein) comprises the step of administering to a subject in need thereof of a therapeutically effective amount of a pharmaceutical composition comprising the composition comprising docetaxel, proteins comprising a human serum albumin, and sodium caprylate as described herein, and a therapeutically effective amount of at least one anti-cancer drug. Examples of an anti-cancer drug include aberaterone, aberaterone acetate, abarelix, aldesleukin, alemtuzumab, alitretinoin, allopurinol, altretamine, anastrozole, arsenic trioxide, asparaginase, azacitidine, bavituximab, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, enzalutamide, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

In some embodiments, a composition comprising docetaxel, proteins comprising a human serum albumin, and sodium caprylate as described herein and an anti-cancer drug are administered simultaneously.

In some embodiments, a composition comprising docetaxel, proteins comprising a human serum albumin, and sodium caprylate as described herein and an anti-cancer drug are administered consecutively.

The composition comprising docetaxel, proteins comprising a human serum albumin, and sodium caprylate described herein can be administered to an individual, such as human, via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the nanoparticle composition is administrated intravenously. In some embodiments, the composition comprising docetaxel, human serum albumin and sodium caprylate described herein can be administered to an individual, such as human, via intravenous route.

The methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of docetaxel will be approximately those already employed in clinical therapies wherein Docetaxel is administered alone or in combination with other chemotherapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for Docetaxel.

Also, provided herein is a composition consisting essentially of docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate. In some embodiments, at least 96% of the proteins in the composition is human serum albumin. In some embodiments, the pH of a composition is neutral (e.g., pH of the composition is from about 5 to about 8, from about 5.5 to about 7.5, or from about 6 to about 7.5, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the docetaxel and the human serum albumin in the composition are in a ratio by weight from about 1:70 to about 1:250. In some embodiments, the docetaxel and the human serum albumin in the composition are in a ratio by weight from about 1:80 to about 1:200. In some embodiments, the docetaxel and the human serum albumin in the composition are in a ratio by weight from about 1:90 to about 1:200. In some embodiments, the docetaxel and the human serum albumin in the composition are in a ratio by weight from about 1:95 to about 1:200. In some embodiments, the docetaxel and the human serum albumin in the composition are in a ratio by weight from about 1:100 to about 1:200. In some embodiments, the docetaxel and the human serum albumin in the composition are in a ratio by weight from about 1:80 to about 1:180. In some embodiments, the docetaxel and the human serum albumin in the composition are in a ratio by weight from about 1:90 to about 1:180. In some embodiments, the docetaxel and the human serum albumin in the composition are in a ratio by weight from about 1:100 to about 1:180. In some embodiments, the docetaxel and the human serum albumin in the composition are in a ratio by weight from about 1:80 to about 1:150. In some embodiments, the docetaxel and the human serum albumin in the composition are in a ratio by weight from about 1:90 to about 1:150. In some embodiments, the docetaxel and the human serum albumin in the composition are in a ratio by weight from about 1:95 to about 1:150. In some embodiments, the docetaxel and the human serum albumin in the composition are in a ratio by weight from about 1:100 to about 1:150. In some embodiments, the docetaxel and the human serum albumin in the composition are in a ratio by weight from about 1:80 to about 1:140. In some embodiments, the docetaxel and the human serum albumin in the composition are in a ratio by weight from about 1:90 to about 1:140. In some embodiments, the docetaxel and the human serum albumin in the composition are in a ratio by weight from about 1:95 to about 1:140. In some embodiments, the docetaxel and the human serum albumin in the composition are in a ratio by weight from about 1:100 to about 1:140. In some embodiments, the docetaxel and the human serum albumin in the composition are in a ratio by weight from about 1:80 to about 1:130. In some embodiments, the docetaxel and the human serum albumin in the composition are in a ratio by weight from about 1:90 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:100 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:95 to about 1:125. In some embodiments, the docetaxel and the human serum albumin in the composition are in a ratio by weight from about 1:100 to about 1:125. In some embodiments, the docetaxel and the human serum albumin in the composition are in a ratio by weight from about 1:90 to about 1:120. In some embodiments, the docetaxel and the human serum albumin in the composition are in a ratio by weight from about 1:100 to about 1:120. In some embodiments, the docetaxel and the human serum albumin in the composition are in a ratio by weight from about 1:90 to about 1:110. In some embodiments, the docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:170, about 1:180, about 1:190, about 1:200, about 1:210, about 1:220, or about 1:250). In some embodiments, the docetaxel and the human serum albumin in the composition are in a ratio by weight of about 1:90. In some embodiments, the docetaxel and the human serum albumin in the composition are in a ratio by weight of about 1:95. In some embodiments, the docetaxel and the human serum albumin in the composition are in a ratio by weight of about 1:100. In some embodiments, the docetaxel and the human serum albumin in the composition are in a ratio by weight of about 1:105. In some embodiments, the docetaxel and the human serum albumin in the composition are in a ratio by weight of about 1:110. In some embodiments, the docetaxel and the human serum albumin in the composition are in a ratio by weight of about 1:115. In some embodiments, the docetaxel and the human serum albumin in the composition are in a ratio by weight of about 1:120. In some embodiments, the docetaxel and the human serum albumin in the composition are in a ratio by weight of about 1:125. In some embodiments, the docetaxel and the human serum albumin in the composition are in a ratio by weight of about 1:130. In some embodiments, the docetaxel and the human serum albumin in the composition are in a ratio by weight of about 1:135. In some embodiments, the docetaxel and the human serum albumin in the composition are in a ratio by weight of about 1:140.

In some embodiments, the human serum albumin is a native human serum albumin. In some embodiments, the human serum albumin is a native human serum albumin obtained from pools of human plasma. In some embodiments, the human serum albumin is a native human serum albumin obtained from pools of human plasma by low temperature controlled fractionation according to the Cohn process.

In some embodiments, the ratio of the amount of sodium caprylate to the amount of the proteins in the composition is from about 0.100 mmol to about 0.200 mmol of sodium caprylate per one gram of the proteins. In some embodiments, the ratio of the amount of sodium caprylate to the amount of the proteins in the composition is from about 0.120 mmol to about 0.190 mmol of sodium caprylate per one gram of the proteins. In some embodiments, the ratio of the amount of sodium caprylate to the amount of the proteins in the composition is from about 0.140 mmol to about 0.180 mmol of sodium caprylate per one gram of the proteins. In some embodiments, the ratio of the amount of sodium caprylate to the amount of the proteins in the composition is from about 0.145 mmol to about 0.175 mmol of sodium caprylate per one gram of the proteins. In some embodiments, the ratio of the amount of sodium caprylate to the amount of the proteins in the composition is from about 0.150 mmol to about 0.170 mmol of sodium caprylate per one gram of the proteins. In some embodiments, the ratio of the amount of sodium caprylate to the amount of the proteins in the composition is about 0.160 mmol of sodium caprylate per one gram of the proteins. In some embodiments, the ratio of the amount of sodium caprylate to the amount of the proteins in the composition is about 0.155 mmol of sodium caprylate per one gram of the proteins. In some embodiments, the ratio of the amount of sodium caprylate to the amount of the proteins in the composition is about 0.165 mmol of sodium caprylate per one gram of the proteins.

In some embodiments, the docetaxel can be a pharmaceutically acceptable salt of docetaxel.

In some embodiments, the docetaxel can be a Docetaxel with 1, 2, or 3 equivalents of the water solvate. In some embodiments, the docetaxel can be a Docetaxel with three equivalents of the water solvate. In some embodiments, docetaxel is the docetaxel trihydrate. In some embodiments, docetaxel is the docetaxel monohydrate. In some embodiments, docetaxel is the docetaxel anhydrous. In some embodiments, the docetaxel can be a docetaxel with one equivalent of the acetone solvate. In some embodiments, the docetaxel can be any one of docetaxel solvates disclosed, for example, in WO2010091650 or US2012007167, the disclosures of which are incorporated herein by reference in its entirety.

In some embodiments, docetaxel is crystalline. In some embodiments, docetaxel is any one of the crystalline forms disclosed, for example, in WO2012115402, U.S. Pat. No. 8,410,294, US20100197944, US20100099897, U.S. Pat. No. 8,357,811, US20100160653, or US20070142457, the disclosures of which are incorporated herein by reference in their entirety.

In some embodiments, docetaxel in amorphous. In some embodiments, Docetaxel is any one of the amorphous forms disclosed, for example, in WO2008102374, the disclosure of which is incorporated herein by reference in its entirety.

As used herein, the term “sodium caprylate” is a compound that has the CAS No. 198 4-06-1. Sodium caprylate can also be called as sodium n-octanoate or sodium octanoate.

As used herein, the term “sodium N-acetyltryptophanate” is a compound that has the CAS No. 62307-74-8. Sodium N-acetyltryptophanate can also be called as sodium N-acetyl-DL-tryptophanate or sodium acetyltryptophanate.

Sodium caprylate and Sodium N-acetyltryptophanate are the stabilizers used in the intravenous Human Albumin (human serum albumin) solution for infusion (e.g. Human Albumin; prepared as a 5%, 20%, or 25% protein solution). These products are pasteurized at 60° C. for at least 10 hours for the inactivation of hepatitis, HIV, or other viruses. Sodium caprylate and Sodium N-acetyltryptophanate are the stabilizers used in pasteurization process.

The intravenous Human Albumin (human serum albumin) for infusion (e.g. Human Albumin; prepared as a 5%, 20%, or 25% protein solution) is a sterile aqueous solution of albumin obtained from large pools of adult human venous plasma by low temperature controlled fractionation according to the Cohn process. Human Albumin solution for infusion is a clear, slightly viscous liquid; it is almost colorless or slightly yellow or green. Human Albumin solution for infusion contains protein, of which at least 96% is human albumin. Human Albumin 5% solution for infusion is mildly hypooncotic to normal human plasma and contains, in each 100 mL, 5 grams of protein, of which at least 96% is human albumin. Human Albumin 25% solution for infusion is hyperoncotic to normal human plasma and contains, in each 100 mL, 25 grams of protein, of which at least 96% is human albumin. The solution contains no preservative.

In some embodiments, the composition is a solid formulation. For example, the solid formulation can be produced in a uniform manner by lyophilization. A skilled artisan would recognize other methods, such as rotary evaporation, that can also produce solid formulations. In some embodiments, the pH of the solid formulation is neutral (e.g., pH of the composition is from about 5 to about 8, from about 5.5 to about 7.5, or from about 6 to about 7.5, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the composition is an aqueous formulation. In some embodiments, the aqueous formulation is substantially free of solvent other than water. In some embodiments, the aqueous formulation is free of solvent other than water. In some embodiments, the pH of the aqueous formulation (e.g., clear aqueous solution) is neutral (e.g., pH of the composition is from about 5 to about 8, from about 5.5 to about 7.5, from about 6 to about 7.5, from about 6.5 to about 7.5, or from about 6 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent. In some embodiments, the aqueous solution contains less than 0.01%, by weight, of any non-water solvent.

In some embodiments, the aqueous formulation can be substantially free of a surfactant selected from the group consisting of CREMOPHOR® surfactants and Polysorbate 80. In some embodiments, the aqueous formulation can be free of a surfactant selected from the group consisting of CREMOPHOR® surfactants and Polysorbate 80.

As used herein, the term “substantially free of surfactant” refers to a formulation containing less than 0.0005%, less than 0.0003%, or less than 0.0001% of surfactants and/or less than 0.0005%, less than 0.0003%, or less than 0.0001% of a surfactant selected from the group consisting of CREMOPHOR® surfactants and Polysorbate 80.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

As used herein, the term “clear aqueous solution” refers to an aqueous solution containing Docetaxel and HSA that is transparent and optically clear upon visual observation and essentially free of visible particles or precipitation of undissolved docetaxel.

The term “essentially free of visible particles or precipitation of undissolved docetaxel” can be assessed as follows: after a clear aqueous solution is filtered with a 0.22 micron filter, the amount of docetaxel in the filtered aqueous solution is at least 95% of the total amount of docetaxel in the aqueous solution before filtration. The total amount of docetaxel in the aqueous solution before filtration includes the particles or precipitation of undissolved docetaxel in the aqueous solution or with the aqueous solution. The amount of the docetaxel in an aqueous solution can be measured by the methods using HPLC. The methods of measuring the amount of the docetaxel in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

As used herein, the term “micron” refers to a unit of measure of one one-thousandth of a millimeter. In some embodiments, the term “micron” refers to a micrometer.

As used herein, the term “aqueous solution” refers to a solution, wherein at least one solvent is water and the weight % of water in the mixture of solvents is at least 50%, at least 60%, at least 70% or at least 90%. In some embodiments, aqueous solution is a solution in which water is the only solvent.

As used herein, the term “aqueous solvent” refers to a liquid comprising water, for example, the liquid comprising at least 50%, at least 60%, at least 70%, at least 90% or at least 95% water. In some embodiments, aqueous solvent is water.

In some embodiments, the aqueous formulation is a clear aqueous solution reconstituted from the solid formulation (e.g. the sterile lyophilized powder) in water. In some embodiments, the aqueous formulation is a clear aqueous solution reconstituted from the solid formulation (e.g. the sterile lyophilized powder) in 0.9% saline solution. In some embodiments, the aqueous formulation is a clear aqueous solution reconstituted from the solid formulation (e.g. the sterile lyophilized powder) in 5% dextrose solution in water.

In some embodiments, the aqueous formulation has pH value from about 5 to about 8. In some embodiments, the aqueous formulation has pH value from about 5.5 to about 7.8. In some embodiments, the aqueous formulation has pH value from about 6 to about 7.5. In some embodiments, the aqueous formulation has pH value from about 6.5 to about 7.5. In some embodiments, the aqueous formulation has pH value from about 6 to about 6.5. In some embodiments, the aqueous formulation has pH value from about 6.5 to about 7. In some embodiments, the aqueous formulation has pH value from about 7 to about 7.5. In some embodiments, the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, after a clear aqueous solution is filtered by a 0.22 micron filter, the amount of docetaxel in the filtered aqueous solution is at least 96% of the total amount of docetaxel in the aqueous solution before filtration. In some embodiments, after a clear aqueous solution is filtered by a 0.22 micron filter, the amount of docetaxel in the filtered aqueous solution is at least 97% of the total amount of docetaxel in the aqueous solution before filtration. In some embodiments, after a clear aqueous solution is filtered by a 0.22 micron filter, the amount of docetaxel in the filtered aqueous solution is at least 98% of the total amount of docetaxel in the aqueous solution before filtration. In some embodiments, after a clear aqueous solution is filtered by a 0.22 micron filter, the amount of docetaxel in the filtered aqueous solution is at least 99% of the total amount of docetaxel in the aqueous solution before filtration. In some embodiments, after a clear aqueous solution is filtered by a 0.22 micron filter, the amount of docetaxel in the filtered aqueous solution is at least 99.5% of the total amount of docetaxel in the aqueous solution before filtration. In some embodiments, the aqueous formulation is substantially free of solvent other than water. In some embodiments, the aqueous formulation is free of solvent other than water.

In some embodiments, after the aqueous formulation (e.g. a clear aqueous solution) is filtered by a 0.22 micron filter, the amount of docetaxel in the filtered aqueous solution is at least 96%, 97%, 98%, 99%, or 99.5% of the total amount of docetaxel in the aqueous solution before filtration, wherein the clear aqueous solution has pH value from about 5 to about 8, and wherein the clear aqueous solution is substantially free of solvent other than water. In some embodiments, after the aqueous formulation (e.g. a clear aqueous solution) is filtered by a 0.22 micron filter, the amount of docetaxel in the filtered aqueous solution is at least 96%, 97%, 98%, 99%, or 99.5% of the total amount of docetaxel in the aqueous solution before filtration, wherein the clear aqueous solution has pH value from about 6 to about 7.5, and wherein the clear aqueous solution is substantially free of solvent other than water.

In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours. In some embodiments, the aqueous formulation is a clear aqueous solution for at least 1 hour, 2 hours, 3 hours, 4 hours, 5 hours, 6 hours, 7 hours, 8 hours, 10 hours, 12 hours, or 24 hours at a temperature from about 20° C. to about 25° C. In some embodiments, the aqueous formulation is a clear aqueous solution for at least 2 hours at a temperature from about 20° C. to about 25° C. In some embodiments, the aqueous formulation is a clear aqueous solution for at least 4 hours at a temperature from about 20° C. to about 25° C. In some embodiments, the aqueous formulation is a clear aqueous solution for at least 5 hours at a temperature from about 20° C. to about 25° C. In some embodiments, the aqueous formulation is a clear aqueous solution for at least 6 hours at a temperature from about 20° C. to about 25° C. In some embodiments, the aqueous formulation is a clear aqueous solution for at least 8 hours at a temperature from about 20° C. to about 25° C. In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours at a temperature from about 1° C. to about 10° C. In some embodiments, the aqueous formulation is a clear aqueous solution for at least 12 hours at a temperature from about 1° C. to about 10° C. In some embodiments, the aqueous formulation is a clear aqueous solution for at least 24 hours at a temperature from about 1° C. to about 10° C. In some embodiments, the aqueous formulation is substantially free of solvent other than water. In some embodiments, the aqueous formulation is free of solvent other than water.

In some embodiments, the concentration of docetaxel in the aqueous formulation is from about 0.1 mg per 1 ml to about 2 mg per 1 ml of the aqueous solvent. In some embodiments, the concentration of docetaxel in the aqueous formulation is from about 0.1 mg per 1 ml to about 1 mg per 1 ml of the aqueous solvent. In some embodiments, the concentration of docetaxel in the aqueous formulation is from about 0.2 mg per 1 ml to about 0.9 mg per 1 ml of the aqueous solvent. In some embodiments, the concentration of docetaxel in the aqueous formulation is from about 0.25 mg per 1 ml to about 0.8 mg per 1 ml of the aqueous solvent. In some embodiments, the concentration of docetaxel in the aqueous formulation is from about 0.3 mg per 1 ml to about 0.75 mg per 1 ml of the aqueous solvent. In some embodiments, the concentration of docetaxel in the aqueous formulation is from about 0.3 mg per 1 ml to about 0.65 mg per 1 ml of the aqueous solvent. In some embodiments, the concentration of docetaxel in the aqueous formulation is from about 0.33 mg per 1 ml to about 0.5 mg per 1 ml of the aqueous solvent. In some embodiments, the concentration of docetaxel in the aqueous formulation is from about 0.2 mg per 1 ml to about 0.3 mg per 1 ml of the aqueous solvent. In some embodiments, the concentration of docetaxel in the aqueous formulation is from about 0.3 mg per 1 ml to about 0.4 mg per 1 ml of the aqueous solvent. In some embodiments, the concentration of docetaxel in the aqueous formulation is from about 0.4 mg per 1 ml to about 0.5 mg per 1 ml of the aqueous solvent. In some embodiments, the concentration of docetaxel in the aqueous formulation is about 0.25 mg per 1 ml of the aqueous solvent. In some embodiments, the concentration of docetaxel in the aqueous formulation is about 0.3 mg per 1 ml of the aqueous solvent. In some embodiments, the concentration of docetaxel in the aqueous formulation is about 0.33 mg per 1 ml of the aqueous solvent. In some embodiments, the concentration of docetaxel in the aqueous formulation is about 0.4 mg per 1 ml of the aqueous solvent. In some embodiments, the concentration of docetaxel in the aqueous formulation is about 0.45 mg per 1 ml of the aqueous solvent. In some embodiments, the concentration of docetaxel in the aqueous formulation is about 0.5 mg per 1 ml of the aqueous solvent. In some embodiments, the aqueous formulation is substantially free of solvent other than water. In some embodiments, the aqueous formulation is free of solvent other than water.

In some embodiments, the aqueous formulation is a clear aqueous solution, wherein the concentration of docetaxel in the aqueous formulation is from about 0.1 mg per 1 ml to about 2 mg per 1 ml of the aqueous solvent. In some embodiments, the aqueous formulation is a clear aqueous solution, wherein the concentration of docetaxel in the aqueous formulation is from about 0.1 mg per 1 ml to about 1 mg per 1 ml of the aqueous solvent. In some embodiments, the aqueous formulation is a clear aqueous solution, wherein the concentration of docetaxel in the aqueous formulation is from about 0.2 mg per 1 ml to about 0.8 mg per 1 ml of the aqueous solvent. In some embodiments, the aqueous formulation is a clear aqueous solution, wherein the concentration of docetaxel in the aqueous formulation is from about 0.3 mg per 1 ml to about 0.75 mg per 1 ml of the aqueous solvent. In some embodiments, the aqueous formulation is a clear aqueous solution, wherein the concentration of docetaxel in the aqueous formulation is from about 0.3 mg per 1 ml to about 0.65 mg per 1 ml of the aqueous solvent. In some embodiments, the aqueous formulation is a clear aqueous solution, wherein the concentration of docetaxel in the aqueous formulation is from about 0.33 mg per 1 ml to about 0.5 mg per 1 ml of the aqueous solvent. In some embodiments, the aqueous formulation is a clear aqueous solution, wherein the concentration of docetaxel in the aqueous formulation is from about 0.25 mg per 1 ml to about 0.35 mg per 1 ml of the aqueous solvent. In some embodiments, the aqueous formulation is a clear aqueous solution, wherein the concentration of docetaxel in the aqueous formulation is from about 0.35 mg per 1 ml to about 0.5 mg per 1 ml of the aqueous solvent. In some embodiments, the aqueous formulation is a clear aqueous solution, wherein the concentration of docetaxel in the aqueous formulation is about 0.3 mg per 1 ml of the aqueous solvent. In some embodiments, the aqueous formulation is a clear aqueous solution, wherein the concentration of docetaxel in the aqueous formulation is about 0.33 mg per 1 ml of the aqueous solvent. In some embodiments, the aqueous formulation is a clear aqueous solution, wherein the concentration of docetaxel in the aqueous formulation is about 0.4 mg per 1 ml of the aqueous solvent. In some embodiments, the aqueous formulation is a clear aqueous solution, wherein the concentration of docetaxel in the aqueous formulation is about 0.5 mg per 1 ml of the aqueous solvent. In some embodiments, the aqueous formulation is substantially free of solvent other than water. In some embodiments, the aqueous formulation is free of solvent other than water.

Also, provided herein is a pharmaceutical composition comprising the composition consisting essentially of docetaxel, proteins comprising a human serum albumin, and sodium caprylate as described herein, and a pharmaceutically acceptable carrier.

Also, provided herein is a method of treating a proliferative disease comprising the step of administering to a subject in need thereof a pharmaceutical composition comprising the composition consisting essentially of docetaxel, proteins comprising a human serum albumin, and sodium caprylate as described herein, and a pharmaceutically acceptable carrier.

Also, provided herein is a method of treating a cancer (e.g., any one of cancers described herein), the method comprising the step of administering to a subject in need thereof of a therapeutically effective amount of a pharmaceutical composition comprising the composition consisting essentially of docetaxel, proteins comprising a human serum albumin, and sodium caprylate as described herein, and a pharmaceutically acceptable carrier.

In some embodiments, the cancer is any one of cancers described herein.

In some embodiments, the cancer is a solid tumor cancer. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma. In some embodiments, the cancer is a breast cancer. In some embodiments, the cancer is a non-small cell lung cancer. In some embodiments, the cancer is a prostate cancer. In some embodiments, the cancer is a gastric cancer. In some embodiments, the cancer is a head and neck cancer. In some embodiments the cancer is an ovarian cancer. In some embodiments, the cancer is a pancreatic cancer. In some embodiments, the cancer is a Kaposi's sarcoma.

The methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of docetaxel will be approximately those already employed in clinical therapies wherein Docetaxel is administered alone or in combination with other chemotherapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for Docetaxel.

Also provided herein is a composition comprising docetaxel, proteins comprising a human serum albumin, and sodium N-acetyltryptophanate, wherein the docetaxel and the human serum albumin in the composition have a ratio by weight from about 1:80 to less than 1:150. In some embodiments, at least 96% of the proteins in the composition is human serum albumin. In some embodiments, the pH of a composition is neutral (e.g., pH of the composition is from about 5 to about 8, from about 5.5 to about 7.5, or from about 6 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the docetaxel and the human serum albumin in the composition are in a ratio by weight from about 1:85 to less than 1:150. In some embodiments, the docetaxel and the human serum albumin in the composition are in a ratio by weight from about 1:90 to less than 1:150. In some embodiments, the docetaxel and the human serum albumin in the composition are in a ratio by weight from about 1:100 to less than 1:150. In some embodiments, the docetaxel and the human serum albumin in the composition are in a ratio by weight from about 1:95 to less than 1:150. In some embodiments, the docetaxel and the human serum albumin in the composition are in a ratio by weight from about 1:110 to less than 1:150. In some embodiments, the docetaxel and the human serum albumin in the composition are in a ratio by weight from about 1:80 to about 1:145. In some embodiments, the docetaxel and the human serum albumin in the composition are in a ratio by weight from about 1:90 to about 1:145. In some embodiments, the docetaxel and the human serum albumin in the composition are in a ratio by weight from about 1:100 to about 1:145. In some embodiments, the docetaxel and the human serum albumin in the composition are in a ratio by weight from about 1:95 to about 1:145. In some embodiments, the docetaxel and the human serum albumin in the composition are in a ratio by weight from about 1:110 to about 1:145. In some embodiments, the docetaxel and the human serum albumin in the composition are in a ratio by weight from about 1:80 to about 1:140. In some embodiments, the docetaxel and the human serum albumin in the composition are in a ratio by weight from about 1:90 to about 1:140. In some embodiments, the docetaxel and the human serum albumin in the composition are in a ratio by weight from about 1:100 to about 1:140. In some embodiments, the docetaxel and the human serum albumin in the composition are in a ratio by weight from about 1:95 to about 1:140. In some embodiments, the docetaxel and the human serum albumin in the composition are in a ratio by weight from about 1:110 to about 1:140. In some embodiments, the docetaxel and the human serum albumin in the composition are in a ratio by weight from about 1:80 to about 1:135. In some embodiments, the docetaxel and the human serum albumin in the composition are in a ratio by weight from about 1:90 to about 1:135. In some embodiments, the docetaxel and the human serum albumin in the composition are in a ratio by weight from about 1:95 to less than 1:135. In some embodiments, the docetaxel and the human serum albumin in the composition are in a ratio by weight from about 1:100 to about 1:135. In some embodiments, the docetaxel and the human serum albumin in the composition are in a ratio by weight from about 1:80 to about 1:130. In some embodiments, the docetaxel and the human serum albumin in the composition are in a ratio by weight from about 1:85 to about 1:130. In some embodiments, the docetaxel and the human serum albumin in the composition are in a ratio by weight from about 1:90 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:100 to about 1:130. In some embodiments, the docetaxel and the human serum albumin in the composition are in a ratio by weight from about 1:80 to about 1:125. In some embodiments, the docetaxel and the human serum albumin in the composition are in a ratio by weight from about 1:85 to about 1:125. In some embodiments, the docetaxel and the human serum albumin in the composition are in a ratio by weight from about 1:90 to about 1:125. In some embodiments, the docetaxel and the human serum albumin in the composition are in a ratio by weight from about 1:95 to about 1:125. In some embodiments, the docetaxel and the human serum albumin in the composition are in a ratio by weight from about 1:100 to about 1:125. In some embodiments, the docetaxel and the human serum albumin in the composition are in a ratio by weight from about 1:80 to about 1:120. In some embodiments, the docetaxel and the human serum albumin in the composition are in a ratio by weight from about 1:90 to about 1:120. In some embodiments, the docetaxel and the human serum albumin in the composition are in a ratio by weight from about 1:95 to about 1:120. In some embodiments, the docetaxel and the human serum albumin in the composition are in a ratio by weight from about 1:100 to about 1:120. In some embodiments, the docetaxel and the human serum albumin in the composition are in a ratio by weight from about 1:80 to about 1:110. In some embodiments, the docetaxel and the human serum albumin in the composition are in a ratio by weight from about 1:90 to about 1:110. In some embodiments, the docetaxel and the human serum albumin in the composition are in a ratio by weight from about 1:90 to about 1:115. In some embodiments, the docetaxel and the human serum albumin in the composition are in a ratio by weight from about 1:90 to about 1:100. In some embodiments, the docetaxel and the human serum albumin in the composition are in a ratio by weight from about 1:100 to about 1:110. In some embodiments, the docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, or about 1:145. In some embodiments, the docetaxel and the human serum albumin in the composition are in a ratio by weight of about 1:90. In some embodiments, the docetaxel and the human serum albumin in the composition are in a ratio by weight of about 1:95. In some embodiments, the docetaxel and the human serum albumin in the composition are in a ratio by weight of about 1:100. In some embodiments, the docetaxel and the human serum albumin in the composition are in a ratio by weight of about 1:105. In some embodiments, the docetaxel and the human serum albumin in the composition are in a ratio by weight of about 1:110. In some embodiments, the docetaxel and the human serum albumin in the composition are in a ratio by weight of about 1:115. In some embodiments, the docetaxel and the human serum albumin in the composition are in a ratio by weight of about 1:120.

In some embodiments, the human serum albumin is a native human serum albumin. In some embodiments, the human serum albumin is a native human serum albumin obtained from pools of human plasma. In some embodiments, the human serum albumin is a native human serum albumin obtained from pools of human plasma by low temperature controlled fractionation according to the Cohn process.

In some embodiments, the composition comprises both sodium N-acetyltryptophanate and sodium caprylate. In some embodiments, the composition comprises both sodium N-acetyltryptophanate and sodium caprylate in a molar ratio of about 1:1. In some embodiments, the composition comprises only sodium N-acetyltryptophanate with no sodium caprylate.

In some embodiments, the composition comprises from about 0.064 mmol to about 0.096 mmol sodium N-acetyltryptophanate and from about 0.064 mmol to about 0.096 mmol sodium caprylate per gram of the proteins in the composition. In some embodiments, the composition comprises from about 0.064 mmol to about 0.096 mmol sodium N-acetyltryptophanate per gram of the proteins in the composition. In some embodiments, the composition comprises from about 0.064 mmol to about 0.096 mmol sodium caprylate per gram of the proteins in the composition. In some embodiments, the composition comprises from about 0.07 mmol to about 0.09 mmol sodium N-acetyltryptophanate and from about 0.07 mmol to about 0.09 mmol sodium caprylate per gram of the proteins in the composition. In some embodiments, the composition comprises from about 0.07 mmol to about 0.09 mmol sodium N-acetyltryptophanate per gram of the proteins in the composition. In some embodiments, the composition comprises from about 0.07 mmol to about 0.09 mmol sodium caprylate per gram of the proteins in the composition. In some embodiments, the composition comprises about 0.08 mmol sodium N-acetyltryptophanate and about 0.08 mmol sodium caprylate per gram of the proteins in the composition. In some embodiments, the composition comprises about 0.08 mmol sodium N-acetyltryptophanate per gram of the proteins in the composition. In some embodiments, the composition comprises about 0.08 mmol sodium caprylate per gram of the proteins in the composition.

In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight from about 1:80 to less than 1:150, and wherein the composition comprises both sodium N-acetyltryptophanate and sodium caprylate. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight from about 1:80 to less than 1:150, and wherein the composition comprises from about 0.064 mmol to about 0.096 mmol sodium N-acetyltryptophanate and from about 0.064 mmol to about 0.096 mmol sodium caprylate per gram of the proteins in the composition. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight from about 1:80 to less than 1:150, and wherein the composition comprises about 0.08 mmol sodium N-acetyltryptophanate and about 0.08 mmol sodium caprylate per gram of the proteins in the composition.

In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight from about 1:90 to about 1:140, and wherein the composition comprises both sodium N-acetyltryptophanate and sodium caprylate. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight from about 1:90 to about 1:140, and wherein the composition comprises from about 0.064 mmol to about 0.096 mmol sodium N-acetyltryptophanate and from about 0.064 mmol to about 0.096 mmol sodium caprylate per gram of the proteins in the composition. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight from about 1:90 to about 1:140, and wherein the composition comprises from about 0.07 mmol to about 0.09 mmol sodium N-acetyltryptophanate and from about 0.07 mmol to about 0.09 mmol sodium caprylate per gram of the proteins in the composition. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight from about 1:90 to about 1:140, and wherein the composition comprises about 0.08 mmol sodium N-acetyltryptophanate and about 0.08 mmol sodium caprylate per gram of the proteins in the composition.

In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, and wherein the composition comprises both sodium N-acetyltryptophanate and sodium caprylate. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, and wherein the composition comprises from about 0.064 mmol to about 0.096 mmol sodium N-acetyltryptophanate and from about 0.064 mmol to about 0.096 mmol sodium caprylate per gram of the proteins in the composition. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, and wherein the composition comprises from about 0.07 mmol to about 0.09 mmol sodium N-acetyltryptophanate and from about 0.07 mmol to about 0.09 mmol sodium caprylate per gram of the proteins in the composition. In some embodiments, provided herein is a composition comprising docetaxel, proteins comprising a human serum albumin, and sodium N-acetyltryptophanate, wherein at least 96% of the proteins in the composition is human serum albumin, wherein the docetaxel and the human serum albumin in the composition have a ratio by weight of about 1:90, about 1:95, 1:100, about 1:105, about 1:110, about 1:115, about 1:120, about 1:125, about 1:130, about 1:135, about 1:140, or about 1:145, and wherein the composition comprises about 0.08 mmol sodium N-acetyltryptophanate and about 0.08 mmol sodium caprylate per gram of the proteins in the composition.

As used herein, the term “proteins” refers to plasma proteins from human plasma. Plasma proteins, also termed blood proteins or serum proteins, are proteins present in human blood plasma.

In some embodiments, the docetaxel can be a pharmaceutically acceptable salt of docetaxel. In some embodiments, the docetaxel can be a Docetaxel with 1, 2, or 3 equivalents of the water solvate. In some embodiments, the docetaxel can be a Docetaxel with three equivalents of the water solvate. In some embodiments, docetaxel is the docetaxel trihydrate. In some embodiments, docetaxel is the docetaxel monohydrate. In some embodiments, docetaxel is the docetaxel anhydrous. In some embodiments, the docetaxel can be a docetaxel with one equivalent of the acetone solvate. In some embodiments, the docetaxel can be any one of docetaxel solvates disclosed, for example, in WO2010091650 or US2012007167, the disclosures of which are incorporated herein by reference in its entirety.

In some embodiments, docetaxel is crystalline. In some embodiments, docetaxel is any one of the crystalline forms disclosed, for example, in WO2012115402, U.S. Pat. No. 8,410,294, US20100197944, US20100099897, U.S. Pat. No. 8,357,811, US20100160653, or US20070142457, the disclosures of which are incorporated herein by reference in their entirety.

In some embodiments, docetaxel in amorphous. In some embodiments, docetaxel is any one of the amorphous forms disclosed, for example, in WO2008102374, the disclosure of which is incorporated herein by reference in its entirety.

As used herein, the term “sodium caprylate” refers to a compound that has the following chemical structure:

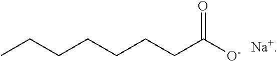

Sodium caprylate can also be called as sodium n-octanoate or sodium octanoate. The CAS Registry No. for sodium caprylate is 1984-06-1.

As used herein, the term “sodium N-acetyltryptophanate” refers to a compound that has the following chemical structure:

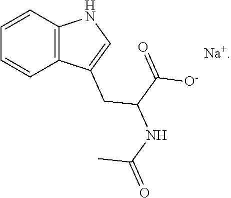 [SMILES: CC(=O)NC(Cc1c[nH]c2ccccc12)C(=O)[O-]]

Sodium N-acetyltryptophanate can also be called as sodium N-acetyl-DL-tryptophanate or sodium acetyltryptophanate. The CAS Registry No. for sodium acetyltryptophanate is 62307-74-8.

In some embodiments, the composition is a solid formulation. For example, the solid formulation can be produced in a uniform manner by lyophilization. A skilled artisan would recognize other methods, such as rotary evaporation, that can also produce solid formulations. In some embodiments, the pH of the solid formulation is neutral (e.g., pH of the composition is from about 5 to about 8, from about 5.5 to about 7.5, or from about 6 to about 7.5, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the composition is an aqueous formulation. In some embodiments, the aqueous formulation is substantially free of solvent other than water. In some embodiments, the aqueous formulation is free of solvent other than water. In some embodiments, the pH of the aqueous formulation (e.g., clear aqueous solution) is neutral (e.g., pH of the composition is from about 5 to about 8, from about 5.5 to about 7.5, from about 6 to about 7.5, from about 6.5 to about 7.5, or from about 6 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent. In some embodiments, the aqueous solution contains less than 0.01%, by weight, of any non-water solvent.

In some embodiments, the aqueous formulation can be substantially free of a surfactant selected from the group consisting of CREMOPHOR® surfactants and Polysorbate 80. In some embodiments, the aqueous formulation can be free of a surfactant selected from the group consisting of CREMOPHOR® surfactants and Polysorbate 80.

As used herein, the term “substantially free of surfactant” refers to a formulation containing less than 0.0005%, less than 0.0003%, or less than 0.0001% of a surfactant selected from the group consisting of CREMOPHOR® surfactants and Polysorbate 80.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

As used herein, the term “clear aqueous solution” refers to an aqueous solution containing Docetaxel and HSA that is transparent and optically clear upon visual observation and essentially free of visible particles or precipitation of undissolved docetaxel.

The term “essentially free of visible particles or precipitation of undissolved docetaxel” can be assessed as follows: after a clear aqueous solution is filtered with a 0.22 micron filter, the amount of docetaxel in the filtered aqueous solution is at least 95% of the total amount of docetaxel in the aqueous solution before filtration. The total amount of docetaxel in the aqueous solution before filtration includes the particles or precipitation of undissolved docetaxel in the aqueous solution or with the aqueous solution. The amount of the docetaxel in an aqueous solution can be measured by the methods using HPLC. The methods of measuring the amount of the docetaxel in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

As used herein, the term “micron” refers to a unit of measure of one one-thousandth of a millimeter. In some embodiments, the term “micron” refers to a micrometer.

As used herein, the term “aqueous solution” refers to a solution, wherein at least one solvent is water and the weight % of water in the mixture of solvents is, for example, at least 50%, at least 60%, at least 70% or at least 90%. In some embodiments, aqueous solution is a solution in which water is the only solvent.

As used herein, the term “aqueous solvent” refers to a liquid comprising at least 50%, at least 60%, at least 70%, at least 90% or at least 95% water. In some embodiments, aqueous solvent is water.

In some embodiments, the aqueous formulation is a clear aqueous solution reconstituted from the solid formulation (e.g. the sterile lyophilized powder) in an aqueous solvent. In some embodiments, the aqueous formulation is a clear aqueous solution reconstituted from the solid formulation (e.g. the sterile lyophilized powder) in water. In some embodiments, the aqueous formulation is a clear aqueous solution reconstituted from the solid formulation (e.g. the sterile lyophilized powder) in 0.9% saline solution. In some embodiments, the aqueous formulation is a clear aqueous solution reconstituted from the solid formulation (e.g. the sterile lyophilized powder) in 5% dextrose solution in water.

In some embodiments, the aqueous formulation is a clear aqueous solution reconstituted from the solid formulation (e.g. the sterile lyophilized powder) in an aqueous solvent, wherein the aqueous formulation has pH value from about 5 to about 8. In some embodiments, the aqueous formulation is a clear aqueous solution reconstituted from the solid formulation (e.g. the sterile lyophilized powder) in water, wherein the aqueous formulation has pH value from about 5 to about 8. In some embodiments, the aqueous formulation is a clear aqueous solution reconstituted from the solid formulation (e.g. the sterile lyophilized powder) in 0.9% saline solution, wherein the aqueous formulation has pH value from about 5 to about 8. In some embodiments, the aqueous formulation is a clear aqueous solution reconstituted from the solid formulation (e.g. the sterile lyophilized powder) in 5% Dextrose water solution, wherein the aqueous formulation has pH value from about 5 to about 8.

In some embodiments, the aqueous formulation is a clear aqueous solution reconstituted from the solid formulation (e.g. the sterile lyophilized powder) in an aqueous solvent, wherein the aqueous formulation has pH value from about 6 to about 7.5. In some embodiments, the aqueous formulation is a clear aqueous solution reconstituted from the solid formulation (e.g. the sterile lyophilized powder) in water, wherein the aqueous formulation has pH value from about 6 to about 7.5. In some embodiments, the aqueous formulation is a clear aqueous solution reconstituted from the solid formulation (e.g. the sterile lyophilized powder) in 0.9% saline solution, wherein the aqueous formulation has pH value from about 6 to about 7.5. In some embodiments, the aqueous formulation is a clear aqueous solution reconstituted from the solid formulation (e.g. the sterile lyophilized powder) in 5% Dextrose water solution, wherein the aqueous formulation has pH value from about 6 to about 7.5.

In some embodiments, the aqueous formulation is a clear aqueous solution reconstituted from the solid formulation (e.g. the sterile lyophilized powder) in an aqueous solvent, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8. In some embodiments, the aqueous formulation is a clear aqueous solution reconstituted from the solid formulation (e.g. the sterile lyophilized powder) in water, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8. In some embodiments, the aqueous formulation is a clear aqueous solution reconstituted from the solid formulation (e.g. the sterile lyophilized powder) in 0.9% saline solution, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8. In some embodiments, the aqueous formulation is a clear aqueous solution reconstituted from the solid formulation (e.g. the sterile lyophilized powder) in 5% dextrose water solution, and wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or about 8.

In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is about 25 mg, about 30 mg, about 35 mg, about 40 mg, about 45 mg, about 50 mg, about 55 mg, about 60 mg, about 65 mg, about 70 mg, about 75 mg, about 80 mg, about 100 mg, about 150 mg, or 200 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 10 mg per 1 ml to about 250 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 25 mg per 1 ml to about 150 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 25 mg per 1 ml to about 100 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 25 mg per 1 ml to about 80 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 30 mg per 1 ml to about 70 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 30 mg per 1 ml to about 50 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 25 mg per 1 ml to about 45 mg per 1 ml of the aqueous solvent. In some aspects of the aforementioned embodiments, the concentration of the reconstituted solid (e.g. the sterile lyophilized powder comprising docetaxel, HSA, sodium N-acetyltryptophanate, and sodium caprylate) in the aqueous formulation is from about 30 mg per 1 ml to about 40 mg per 1 ml of the aqueous solvent.

In some embodiments, the aqueous formulation has pH value from about 5 to about 8. In some embodiments, the aqueous formulation has pH value from about 5.5 to about 7.8. In some embodiments, the aqueous formulation has pH value from about 6 to about 7.5. In some embodiments, the aqueous formulation has pH value from about 6.5 to about 7.5. In some embodiments, the aqueous formulation has pH value from about 6 to about 6.5. In some embodiments, the aqueous formulation has pH value from about 6.5 to about 7. In some embodiments, the aqueous formulation has pH value from about 7 to about 7.5. In some embodiments, the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, the aqueous formulation is a clear aqueous solution, wherein the aqueous formulation has pH value from about 5 to about 8, and wherein the aqueous formulation is substantially free of solvent other than water. In some embodiments, the aqueous formulation is a clear aqueous solution, wherein the aqueous formulation has pH value from about 6 to about 7.5, and wherein the aqueous formulation is substantially free of solvent other than water.

In some embodiments, after a clear aqueous solution is filtered by a 0.22 micron filter, the amount of docetaxel in the filtered aqueous solution is at least 96% of the total amount of docetaxel in the aqueous solution before filtration. In some embodiments, after a clear aqueous solution is filtered by a 0.22 micron filter, the amount of docetaxel in the filtered aqueous solution is at least 97% of the total amount of docetaxel in the aqueous solution before filtration. In some embodiments, after a clear aqueous solution is filtered by a 0.22 micron filter, the amount of docetaxel in the filtered aqueous solution is at least 98% of the total amount of docetaxel in the aqueous solution before filtration. In some embodiments, after a clear aqueous solution is filtered by a 0.22 micron filter, the amount of docetaxel in the filtered aqueous solution is at least 99% of the total amount of docetaxel in the aqueous solution before filtration. In some embodiments, after a clear aqueous solution is filtered by a 0.22 micron filter, the amount of docetaxel in the filtered aqueous solution is at least 99.5% of the total amount of docetaxel in the aqueous solution before filtration. In some embodiments, the aqueous formulation is substantially free of solvent other than water. In some embodiments, the aqueous formulation is free of solvent other than water.

In some embodiments, after the aqueous formulation (e.g. a clear aqueous solution) is filtered by a 0.22 micron filter, the amount of docetaxel in the filtered aqueous solution is at least about 96%, about 97%, about 98%, about 99%, or about 99.5% of the total amount of docetaxel in the aqueous solution before filtration, wherein the clear aqueous solution has pH value from about 5 to about 8, and wherein the clear aqueous solution is substantially free of solvent other than water. In some embodiments, after the aqueous formulation (e.g. a clear aqueous solution) is filtered by a 0.22 micron filter, the amount of docetaxel in the filtered aqueous solution is at least about 96%, about 97%, about 98%, about 99%, or about 99.5% of the total amount of docetaxel in the aqueous solution before filtration, wherein the clear aqueous solution has pH value from about 6 to about 7.5, and wherein the clear aqueous solution is substantially free of solvent other than water.

In some embodiments, the aqueous formulation is a clear aqueous solution for at least about 1 hour, about 2 hours, about 3 hours, about 4 hours, about 5 hours, about 6 hours, about 7 hours, about 8 hours, about 10 hours, about 12 hours, about 24 hours, or 72 hours. In some embodiments, the aqueous formulation is a clear aqueous solution for at least 1 hour, 2 hours, 3 hours, 4 hours, 5 hours, 6 hours, 7 hours, 8 hours, 10 hours, 12 hours, or about 24 hours at a temperature from about 20° C. to about 25° C. In some embodiments, the aqueous formulation is a clear aqueous solution for at least 2 hours at a temperature from about 20° C. to about 25° C. In some embodiments, the aqueous formulation is a clear aqueous solution for at least 3 hours at a temperature from about 20° C. to about 25° C. In some embodiments, the aqueous formulation is a clear aqueous solution for at least 4 hours at a temperature from about 20° C. to about 25° C. In some embodiments, the aqueous formulation is a clear aqueous solution for at least 5 hours at a temperature from about 20° C. to about 25° C. In some embodiments, the aqueous formulation is a clear aqueous solution for at least 6 hours at a temperature from about 20° C. to about 25° C. In some embodiments, the aqueous formulation is a clear aqueous solution for at least 8 hours at a temperature from about 20° C. to about 25° C. In some embodiments, the aqueous formulation is a clear aqueous solution for at least about 1 hour, about 2 hours, about 3 hours, about 4 hours, about 5 hours, about 6 hours, about 7 hours, about 8 hours, about 10 hours, about 12 hours, about 24 hours, or about 72 hours at a temperature from about 1° C. to about 10° C. In some embodiments, the aqueous formulation is a clear aqueous solution for at least 12 hours at a temperature from about 1° C. to about 10° C. In some embodiments, the aqueous formulation is a clear aqueous solution for at least 24 hours at a temperature from about 1° C. to about 10° C. In some embodiments, the aqueous formulation is substantially free of solvent other than water. In some embodiments, the aqueous formulation is free of solvent other than water.

In some embodiments, the aqueous formulation is a clear aqueous solution for at least 2 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 3 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 4 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 5 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 6 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 8 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 12 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 24 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is substantially free of solvent other than water. In some embodiments, the aqueous formulation is free of solvent other than water.

In some embodiments, the concentration of docetaxel in the aqueous formulation is from about 0.1 mg per 1 ml to about 2 mg per 1 ml of the aqueous solvent. In some embodiments, the concentration of docetaxel in the aqueous formulation is from about 0.1 mg per 1 ml to about 1 mg per 1 ml of the aqueous solvent. In some embodiments, the concentration of docetaxel in the aqueous formulation is from about 0.2 mg per 1 ml to about 0.9 mg per 1 ml of the aqueous solvent. In some embodiments, the concentration of docetaxel in the aqueous formulation is from about 0.25 mg per 1 ml to about 0.8 mg per 1 ml of the aqueous solvent. In some embodiments, the concentration of docetaxel in the aqueous formulation is from about 0.3 mg per 1 ml to about 0.75 mg per 1 ml of the aqueous solvent. In some embodiments, the concentration of docetaxel in the aqueous formulation is from about 0.3 mg per 1 ml to about 0.65 mg per 1 ml of the aqueous solvent. In some embodiments, the concentration of docetaxel in the aqueous formulation is from about 0.33 mg per 1 ml to about 0.5 mg per 1 ml of the aqueous solvent. In some embodiments, the concentration of docetaxel in the aqueous formulation is from about 0.2 mg per 1 ml to about 0.3 mg per 1 ml of the aqueous solvent. In some embodiments, the concentration of docetaxel in the aqueous formulation is from about 0.3 mg per 1 ml to about 0.4 mg per 1 ml of the aqueous solvent. In some embodiments, the concentration of docetaxel in the aqueous formulation is from about 0.4 mg per 1 ml to about 0.5 mg per 1 ml of the aqueous solvent. In some embodiments, the concentration of docetaxel in the aqueous formulation is about 0.25 mg per 1 ml of the aqueous solvent. In some embodiments, the concentration of docetaxel in the aqueous formulation is about 0.3 mg per 1 ml of the aqueous solvent. In some embodiments, the concentration of docetaxel in the aqueous formulation is about 0.33 mg per 1 ml of the aqueous solvent. In some embodiments, the concentration of docetaxel in the aqueous formulation is about 0.4 mg per 1 ml of the aqueous solvent. In some embodiments, the concentration of docetaxel in the aqueous formulation is about 0.45 mg per 1 ml of the aqueous solvent. In some embodiments, the concentration of docetaxel in the aqueous formulation is about 0.5 mg per 1 ml of the aqueous solvent. In some embodiments, the aqueous formulation is substantially free of solvent other than water. In some embodiments, the aqueous formulation is free of solvent other than water.

In some embodiments, the aqueous formulation is a clear aqueous solution, wherein the concentration of docetaxel in the aqueous formulation is from about 0.1 mg per 1 ml to about 2 mg per 1 ml of the aqueous solvent. In some embodiments, the aqueous formulation is a clear aqueous solution, wherein the concentration of docetaxel in the aqueous formulation is from about 0.1 mg per 1 ml to about 1 mg per 1 ml of the aqueous solvent. In some embodiments, the aqueous formulation is a clear aqueous solution, wherein the concentration of docetaxel in the aqueous formulation is from about 0.2 mg per 1 ml to about 0.8 mg per 1 ml of the aqueous solvent. In some embodiments, the aqueous formulation is a clear aqueous solution, wherein the concentration of docetaxel in the aqueous formulation is from about 0.3 mg per 1 ml to about 0.75 mg per 1 ml of the aqueous solvent. In some embodiments, the aqueous formulation is a clear aqueous solution, wherein the concentration of docetaxel in the aqueous formulation is from about 0.3 mg per 1 ml to about 0.65 mg per 1 ml of the aqueous solvent. In some embodiments, the aqueous formulation is a clear aqueous solution, wherein the concentration of docetaxel in the aqueous formulation is from about 0.33 mg per 1 ml to about 0.5 mg per 1 ml of the aqueous solvent. In some embodiments, the aqueous formulation is a clear aqueous solution, wherein the concentration of docetaxel in the aqueous formulation is from about 0.25 mg per 1 ml to about 0.45 mg per 1 ml of the aqueous solvent. In some embodiments, the aqueous formulation is a clear aqueous solution, wherein the concentration of docetaxel in the aqueous formulation is from about 0.25 mg per 1 ml to about 0.35 mg per 1 ml of the aqueous solvent. In some embodiments, the aqueous formulation is a clear aqueous solution, wherein the concentration of docetaxel in the aqueous formulation is from about 0.35 mg per 1 ml to about 0.5 mg per 1 ml of the aqueous solvent. In some embodiments, the aqueous formulation is a clear aqueous solution, wherein the concentration of docetaxel in the aqueous formulation is from about 0.3 mg per 1 ml to about 0.4 mg per 1 ml of the aqueous solvent. In some embodiments, the aqueous formulation is a clear aqueous solution, wherein the concentration of docetaxel in the aqueous formulation is about 0.3 mg per 1 ml of the aqueous solvent. In some embodiments, the aqueous formulation is a clear aqueous solution, wherein the concentration of docetaxel in the aqueous formulation is about 0.33 mg per 1 ml of the aqueous solvent. In some embodiments, the aqueous formulation is a clear aqueous solution, wherein the concentration of docetaxel in the aqueous formulation is about 0.4 mg per 1 ml of the aqueous solvent. In some embodiments, the aqueous formulation is a clear aqueous solution, wherein the concentration of docetaxel in the aqueous formulation is about 0.5 mg per 1 ml of the aqueous solvent. In some embodiments, the aqueous formulation is substantially free of solvent other than water. In some embodiments, the aqueous formulation is free of solvent other than water.

In some embodiments, the aqueous formulation is a clear aqueous solution, wherein the concentration of docetaxel in the aqueous formulation is from about 0.1 mg per 1 ml to about 1 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.2 mg per 1 ml to about 0.8 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.3 mg per 1 ml to about 0.75 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.3 mg per 1 ml to about 0.65 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.33 mg per 1 ml to about 0.5 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.25 mg per 1 ml to about 0.45 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.25 mg per 1 ml to about 0.35 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is from about 0.3 mg per 1 ml to about 0.4 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.25 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.3 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.33 mg per 1 ml of the aqueous solvent, and wherein the aqueous formulation is substantially free of solvent other than water. In some embodiments, the aqueous formulation is a clear aqueous solution, wherein the concentration of docetaxel in the aqueous formulation is about 0.4 mg per 1 ml of the aqueous solvent, and wherein the aqueous formulation is substantially free of solvent other than water.

In some embodiments, provided herein is a composition comprising docetaxel, proteins comprising a human serum albumin, and sodium N-acetyltryptophanate, wherein the docetaxel and the human serum albumin in the composition have a ratio by weight from about 1:80 to less than about 1:150, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.1 mg per 1 ml to about 1 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight from about 1:90 to about 1:140, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.1 mg per 1 ml to about 1 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.1 mg per 1 ml to about 1 mg per 1 ml of the aqueous solvent, and wherein the aqueous formulation is substantially free of solvent other than water.

In some embodiments, provided herein is a composition comprising docetaxel, proteins comprising a human serum albumin, and sodium N-acetyltryptophanate, wherein the docetaxel and the human serum albumin in the composition have a ratio by weight from about 1:80 to less than about 1:150, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.25 mg per 1 ml to about 0.65 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight from about 1:90 to about 1:140, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.25 mg per 1 ml to about 0.65 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight of about 1:90, about 1:95, 1:100, about 1:105, about 1:110, about 1:115, about 1:120, about 1:125, about 1:130, about 1:135, about 1:140, or about 1:145, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.25 mg per 1 ml to about 0.65 mg per 1 ml of the aqueous solvent, and wherein the aqueous formulation is substantially free of solvent other than water.

In some embodiments, provided herein is a composition comprising docetaxel, proteins comprising a human serum albumin, and sodium N-acetyltryptophanate, wherein the docetaxel and the human serum albumin in the composition have a ratio by weight from about 1:80 to less than about 1:150, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.3 mg per 1 ml to about 0.45 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight from about 1:90 to about 1:140, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.3 mg per 1 ml to about 0.45 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight of about 1:90, about 1:95, 1:100, about 1:105, about 1:110, about 1:115, about 1:120, about 1:125, about 1:130, about 1:135, about 1:140, or about 1:145, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.3 mg per 1 ml to about 0.45 mg per 1 ml of the aqueous solvent, and wherein the aqueous formulation is substantially free of solvent other than water.

In some embodiments, provided herein is a composition comprising docetaxel, proteins comprising a human serum albumin, and sodium N-acetyltryptophanate, wherein the docetaxel and the human serum albumin in the composition have a ratio by weight from about 1:80 to less than about 1:150, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is about 0.25 mg per 1 ml, about 0.3 mg per 1 ml, about 0.33 mg per 1 ml, about 0.4 mg per 1 ml, or about 0.5 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight from about 1:90 to about 1:140, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is about 0.25 mg per 1 ml, about 0.3 mg per 1 ml, about 0.33 mg per 1 ml, about 0.4 mg per 1 ml, or about 0.5 mg per 1 ml of the aqueous solvent, and wherein the aqueous formulation is substantially free of solvent other than water. In some embodiments, provided herein is a composition comprising docetaxel, proteins comprising a human serum albumin, and sodium N-acetyltryptophanate, wherein the docetaxel and the human serum albumin in the composition have a ratio by weight of about 1:90, about 1:95, about 1:100, about 1:105, about 1:110, about 1:115, about 1:120, about 1:125, about 1:130, about 1:135, about 1:140, or about 1:145, and wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is about 0.25 mg per 1 ml, about 0.3 mg per 1 ml, about 0.33 mg per 1 ml, about 0.4 mg per 1 ml, or about 0.5 mg per 1 ml of the aqueous solvent, and wherein the aqueous formulation is substantially free of solvent other than water.

In some embodiments, provided herein is a composition comprising docetaxel, proteins comprising a human serum albumin, sodium N-acetyltryptophanate, and sodium caprylate, wherein at least 96% of the proteins in the composition is human serum albumin, wherein the docetaxel and the human serum albumin in the composition have a ratio by weight from about 1:80 to less than about 1:150, wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.3 mg per 1 ml to about 0.4 mg per 1 ml of the aqueous solvent, and wherein the aqueous formulation is substantially free of solvent other than water. In some embodiments, provided herein is a composition comprising docetaxel, proteins comprising a human serum albumin, sodium N-acetyltryptophanate, and sodium caprylate, wherein at least 96% of the proteins in the composition is human serum albumin, wherein the docetaxel and the human serum albumin in the composition have a ratio by weight from about 1:90 to about 1:140, wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.3 mg per 1 ml to about 0.4 mg per 1 ml of the aqueous solvent, and wherein the aqueous formulation is substantially free of solvent other than water. In some embodiments, provided herein is a composition comprising docetaxel, proteins comprising a human serum albumin, sodium N-acetyltryptophanate, and sodium caprylate, wherein at least 96% of the proteins in the composition is human serum albumin, wherein the docetaxel and the human serum albumin in the composition have a ratio by weight of about 80, about 85, about 1:90, about 1:95, about 1:100, about 1:105, about 1:110, about 1:115, about 1:120, about 1:125, about 1:130, about 1:135, about 1:140, or about 1:145, wherein the composition comprises from about 0.064 mmol to about 0.096 mmol sodium N-acetyltryptophanate and from about 0.064 mmol to about 0.096 mmol sodium caprylate per gram of the proteins in the composition, wherein the composition is an aqueous formulation, wherein the concentration of docetaxel in the aqueous formulation is from about 0.3 mg per 1 ml to about 0.4 mg per 1 ml of the aqueous solvent, and wherein the aqueous formulation is substantially free of solvent other than water.

Also, provided herein is a pharmaceutical composition comprising the composition comprising docetaxel, proteins comprising a human serum albumin, and sodium N-acetyltryptophanate as described herein, and a pharmaceutically acceptable carrier.

In some embodiments, the pharmaceutical composition further comprises at least one anti-cancer drug (e.g., any one of the anti-cancer drugs as described herein).

In some embodiments, the pharmaceutical composition is free of a surfactant selected from the group consisting of CREMOPHOR® surfactants and Polysorbate 80. In some embodiments, the pharmaceutical composition is substantially free of a surfactant selected from the group consisting of CREMOPHOR® surfactants and Polysorbate 80.

Also, provided herein is a method of treating a cancer, the method comprising the step of administering to a subject in need thereof of a therapeutically effective amount of a pharmaceutical composition comprising the composition comprising the composition comprising docetaxel, proteins comprising a human serum albumin, and sodium N-acetyltryptophanate as described herein, and a pharmaceutically acceptable carrier.

In some embodiments, the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

In some embodiments, cancer is selected from sarcoma, angiosarcoma, fibrosarcoma, rhabdomyosarcoma, liposarcoma, myxoma, rhabdomyoma, fibroma, lipoma, teratoma, non-small cell lung cancer (NSCLC), bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

In some embodiments, the cancer is a solid tumor cancer. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma. In some embodiments, the cancer is a breast cancer. In some embodiments, the cancer is a non-small cell lung cancer. In some embodiments, the cancer is a prostate cancer. In some embodiments, the cancer is a gastric cancer. In some embodiments, the cancer is a head and neck cancer. In some embodiments the cancer is an ovarian cancer. In some embodiments, the cancer is a pancreatic cancer. In some embodiments, the cancer is a Kaposi's sarcoma.

In some embodiments, the method of treating cancer (e.g., any one of cancers described herein) comprises the step of administering to a subject in need thereof a therapeutically effective amount of a pharmaceutical composition comprising the composition comprising docetaxel, proteins comprising a human serum albumin, and sodium N-acetyltryptophanate as described herein, and a therapeutically effective amount of at least one inhibitor of the kinases for the treatment of cancer described herein.

In some embodiments, the method of treating cancer (e.g. any one of cancers described herein) comprises the step of administering to a subject in need thereof a therapeutically effective amount of a pharmaceutical composition comprising the composition comprising docetaxel, proteins comprising a human serum albumin, and sodium N-acetyltryptophanate as described herein, and a therapeutically effective amount of at least one anti-cancer drug described herein.

In some embodiments, a composition comprising docetaxel, proteins comprising a human serum albumin, and sodium N-acetyltryptophanate as described herein and an anti-cancer drug are administered simultaneously.

In some embodiments, a composition comprising docetaxel, proteins comprising a human serum albumin, and sodium N-acetyltryptophanate as described herein and an anti-cancer drug are administered consecutively.

The composition comprising docetaxel, proteins comprising a human serum albumin, and sodium N-acetyltryptophanate described herein can be administered to an individual, such as human, via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the nanoparticle composition is administrated intravenously. In some embodiments, the composition comprising docetaxel, human serum albumin and sodium N-acetyltryptophanate described herein can be administered to an individual, such as human, via intravenous route.

The methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of docetaxel will be approximately those already employed in clinical therapies wherein Docetaxel is administered alone or in combination with other chemotherapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for Docetaxel.

### Kits

The present invention also includes pharmaceutical kits useful, for example, in the treatment or prevention of any one of diseases or disorders referred to herein, which include one or more containers containing a pharmaceutical composition comprising a composition of docetaxel and the human serum albumin as described herein. Such kits can further include, if desired, one or more of various conventional pharmaceutical kit components, such as, for example, containers with one or more pharmaceutically acceptable carriers (e.g., water, 0.9% saline, or 5% dextrose), additional containers, etc., as will be readily apparent to those skilled in the art. Instructions, either as inserts or as labels, indicating quantities of the components to be administered (e.g., dosage amounts as described herein), guidelines for administration, and/or guidelines for mixing the components, can also be included in the kit.

### Methods of Making

Also, provided herein are several methods to prepare a composition comprising docetaxel, proteins comprising a human serum albumin, and sodium caprylate as described herein, or a composition comprising docetaxel, proteins comprising a human serum albumin, and sodium N-acetyltryptophanate as described herein.

In some embodiments, the present disclosure provides a method of preparing a composition comprising docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate.

In some embodiments, the present disclosure provides a method of preparing a composition comprising docetaxel, proteins comprising a human serum albumin, and sodium N-acetyltryptophanate, wherein the docetaxel and the human serum albumin in the composition have a ratio by weight from about 1:80 to less than 1:150.

In some embodiments, the present disclosure provides a method of preparing a composition comprising docetaxel, proteins comprising a human serum albumin, sodium caprylate, and sodium N-acetyltryptophanate, wherein the docetaxel and the human serum albumin in the composition have a ratio by weight from about 1:80 to less than 1:150.

In some embodiments, at least 96% of the proteins in the composition is human serum albumin.

In some embodiments, the method comprises mixing an organic solution of docetaxel in a polar water-miscible organic solvent and a first aqueous solution containing human serum albumin to form a second aqueous solution, wherein the second aqueous solution is a clear aqueous solution.

In some embodiments, the method further comprises removing said polar water-miscible organic solvent and water from the second aqueous solution.

A non-limiting preferred method is as follows.

### Formation of the Organic Solution

In some embodiments, docetaxel is dissolved in a polar organic solvent (e.g., an alcohol such as methanol, ethanol, isopropanol, and/or n-butanol; THF, CH₃CN; DMF; or mixtures thereof) to form an organic solution.

As used herein, the term “organic solution” refers to a solution wherein at least one solvent is a non-aqueous solvent and the weight % of the non-aqueous solvent in the mixture of solvents is at least 50%, at least 60%, at least 70% or at least 90%. In some embodiments, organic solution is a solution in which does not comprise water as a solvent.

In some embodiments, the terms “organic solvent” and “non-aqueous solvent” are used interchangeably and refer to a liquid comprising is at least 50%, at least 60%, at least 70%, at least 90%, or at least 95% of a solvent other than water.

The polar organic solvent is miscible in water. In some embodiments, the polar organic solvent is an alcohol. In some embodiments, the polar organic solvent is ethanol or methanol, or mixtures thereof. In some embodiments, the polar organic solvent can be ethanol. In some embodiments, the polar organic solvent is methanol.

In some embodiments, the amount of polar organic solvent is from about 0.005 mL to about 10 mL per 1 mg of docetaxel. In some embodiments, the amount of polar organic solvent is from about 0.01 mL to about 5 mL per 1 mg of docetaxel. In some embodiments, the amount of polar organic solvent is from about 0.05 mL to about 5 mL per 1 mg of docetaxel. In some embodiments, the amount of polar organic solvent is from about 0.1 mL to about 2.0 mL per 1 mg of docetaxel.

### Formation of the First Aqueous Solution

In some embodiments, a defined amount of human serum albumin is dissolved in an amount of water to form a first aqueous solution.

In some embodiments, the first aqueous solution is an intravenous Human Albumin (human serum albumin) solution for infusion (e.g. Human Albumin USP; prepared as a 5%, 20%, or 25% protein solution). In some embodiments, the first aqueous solution is prepared from an intravenous Human Albumin (human serum albumin) solution for infusion (e.g. Human Albumin USP; prepared as a 5%, 20%, or 25% protein solution). In some embodiments, the first aqueous solution is prepared from an intravenous Human Albumin (human serum albumin) solution for infusion (e.g. Human Albumin USP; prepared as a 5%, 20%, or 25% protein solution) by diluted with water.

In some embodiments, the first aqueous solution is an intravenous Human Albumin (human serum albumin) solution for infusion (e.g. Human Albumin; prepared as a 5%, 20%, or 25% protein solution) with sodium caprylate as stabilizer. In some embodiments, the first aqueous solution is prepared from an intravenous Human Albumin (human serum albumin) solution for infusion (e.g. Human Albumin; prepared as a 5%, 20%, or 25% protein solution) with sodium caprylate as stabilizer. In some embodiments, the first aqueous solution is prepared from an intravenous Human Albumin (human serum albumin) solution for infusion (e.g. Human Albumin; prepared as a 5%, 20%, or 25% protein solution) with sodium caprylate as stabilizer by diluted with water. In some embodiments, the first aqueous solution is an intravenous Human Albumin (human serum albumin) solution for infusion (e.g. Human Albumin USP; prepared as a 5%, 20%, or 25% protein solution) with sodium caprylate and sodium N-acetyltryptophanate as stabilizers. In some embodiments, the first aqueous solution is prepared from an intravenous Human Albumin (human serum albumin) solution for infusion (e.g. Human Albumin USP; prepared as a 5%, 20%, or 25% protein solution) with sodium caprylate and sodium N-acetyltryptophanate as stabilizers. In some embodiments, the first aqueous solution is prepared from an intravenous Human Albumin (human serum albumin) solution for infusion (e.g. Human Albumin; prepared as a 5%, 20%, or 25% protein solution) with sodium caprylate and sodium N-acetyltryptophanate as stabilizers by diluted with water.

In some embodiments, the amount of aqueous solvent (e.g., water, saline, dextrose solution, or a buffer (e.g., any one of buffers described herein)) to prepare the first aqueous solution is from about 1 mL to about 1000 L, from about 5 mL to about 500 L, from about 10 mL to about 100 L, from about 100 mL to about 50 L, from about 200 mL to about 20 L, or from about 1 L to about 10 L.

In some embodiments, the amount of HSA prepare the first aqueous solution is from about 100 mg to about 500 kg, from about 150 mg to about 100 kg, from about 200 mg to about 10 kg, from about 300 mg to about 5 kg, from about 400 mg to about 2 kg, or from about 500 mg to about 1 kg.

In some embodiments, the amount of aqueous solvent in the first aqueous solution is from about 0.002 mL to about 10 mL per 1 mg of human serum albumin. In some embodiments, the amount of aqueous solvent in the first aqueous solution is from about 0.005 mL to about 1 mL per 1 mg of human serum albumin. In some embodiments, the amount of aqueous solvent in the first aqueous solution is from about 0.01 mL to about 0.2 mL per 1 mg of human serum albumin. In some embodiments, the amount of aqueous solvent in the first aqueous solution is from about 0.01 mL to about 0.1 mL per 1 mg of human serum albumin. In some embodiments, the amount of aqueous solvent in the first aqueous solution is from about 0.01 mL to about 0.05 mL per 1 mg of human serum albumin. In some embodiments, the amount of aqueous solvent in the first aqueous solution is from about 0.01 mL to about 0.02 mL per 1 mg of human serum albumin. In some embodiments, the amount of aqueous solvent in the first aqueous solution is about 0.01 mL, about 0.015 mL, about 0.02 mL, about 0.025 mL, about 0.03 mL, about 0.035 mL, about 0.04 mL, about 0.045 mL, or about 0.05 mL per 1 mg of human serum albumin.

In some embodiments, the resulting composition comprising docetaxel, proteins comprising a human serum albumin, and sodium caprylate can have any molar ratio or any ratio by weight of the docetaxel to the human serum albumin as described herein.

In some embodiments, the resulting composition comprising docetaxel, proteins comprising a human serum albumin, and sodium N-acetyltryptophanate can have any molar ratio or any ratio by weight of the docetaxel to the human serum albumin as described herein.

In some embodiments, the resulting composition comprising docetaxel, proteins comprising a human serum albumin, sodium N-acetyltryptophanate, and sodium caprylate can have any molar ratio or any ratio by weight of the docetaxel to the human serum albumin as described herein.

In some embodiments, the preparation of the organic solution and the preparation of the first aqueous solution are performed concurrently.

In some embodiments, the preparation of the organic solution and the preparation of the first aqueous solution are performed sequentially. In some embodiments, the preparation of the organic solution is performed before the preparation of the first aqueous solution. In some embodiments, the preparation of the first aqueous solution is performed before the preparation of the organic solution.

In some embodiments, the range of pH in the first aqueous solution is from about 3 to about 9, from about 4 to about 8, from about 5 to about 8, from about 5 to about 7, from about 6 to about 7, from about 3 to about 5, from about 3 to about 7, from about 4 to about 6, or from about 6 to about 6.5. In some embodiments, the pH of the first aqueous solution is about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, or about 8.

### Formation of the Second Aqueous Solution

In some embodiments, the organic solution of docetaxel is mixed with the first aqueous solution of human serum albumin to form a second aqueous solution. In some embodiments, the second aqueous solution is a clear aqueous solution.

In some embodiments, the volume ratio of the amount of water to the amount of the polar organic solvent is in a range from about 1:1 to about 1000:1. In some embodiments, the volume ratio of the amount of water to the amount of the polar organic solvent is in a range from about 1.5:1 to about 100:1. In some embodiments, the volume ratio of the amount of water to the amount of the polar organic solvent is in a range from about 1.5:1 to about 20:1. In some embodiments, the volume ratio of the amount of water to the amount of the polar organic solvent is in a range from about 1.5:1 to about 10:1. In some embodiments, the volume ratio of the amount of water to the amount of the polar organic solvent is in a range from about 2:1 to about 10:1. In some embodiments, the volume ratio of the amount of water to the amount of the polar organic solvent is about 1.5:1, about 2:1, about 2.2:1, about 2.3:1, about 2.4:1, about 2.5:1, about 3:1, about 4:1, about 5:1, about 6:1, about 7:1, about 8:1, about 9:1, or about 10:1.

In some embodiments, the organic solution is added to the first aqueous solution to form a second aqueous solution. In some embodiments, the organic solution is added dropwise to the first aqueous solution to form a second aqueous solution. In some embodiments, the first aqueous solution is added to the organic solution to form a second aqueous solution. In some embodiments, the mixing is performed with agitation. In some embodiments, the mixing is performed with stirring. In some embodiments, the mixing is performed with shaking.

In some embodiments, the addition is done at the temperature from about 0° C. to about 35° C. In some embodiments, the addition is done at the temperature from about 0° C. to about 25° C. In some embodiments, the addition is done at the temperature from about 0° C. to about 10° C. In some embodiments, the addition is done at the temperature from about 0° C. to about 5° C. In some embodiments, the addition is done at the temperature about 0° C. In some embodiments, the addition is done at the temperature about 1° C. In some embodiments, the addition is done at the temperature about 2° C. In some embodiments, the addition is done at the temperature about 3° C. In some embodiments, the addition is done at the temperature about 4° C. In some embodiments, the addition is done at the temperature about 5° C. In some embodiments, the addition is done at the temperature about 10° C.

In some embodiments, the time of addition is in a range from about 0.1 min to about 24 hours. In some embodiments, the time of addition is in a range from about 1 min to about 2 hour. In some embodiments, the time of addition is in a range from about 1 min to about 1 hour. In some embodiments, the time of addition is in a range from about 5 min to about 30 min.

In some embodiments, the range of pH in the first aqueous solution is from about 3 to about 9, from about 4 to about 8, from about 5 to about 8, from about 5 to about 7, from about 6 to about 7, from about 3 to about 5, from about 3 to about 7, from about 4 to about 6, or from about 6 to about 6.5. In some embodiments, the pH of the first aqueous solution is about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, or about 8.

### Removal of Organic Solvent

In some embodiments, upon completion of mixing of the organic solution with the first aqueous solution to form the second aqueous solution, the polar organic solvent is removed from the second aqueous solution.

In some embodiments, the polar organic solvent is removed under reduced pressure. In some embodiments, the polar organic solvent is removed using rotary evaporation. In some embodiments, the polar organic solvent is removed under a vacuum.

In some embodiments, the removal of the polar organic solvent yields a clear aqueous solution. In some embodiments, water is removed from the aqueous under a vacuum. In some embodiments, water is removed from the aqueous solution using rotary evaporation. In some embodiments, water is removed from the aqueous solution by lyophilization.

In some embodiments, the solvents including both water and organic solvent are removed from the second aqueous solution simultaneously to provide a solid composition. In some embodiments, the solvents are removed under a vacuum. In some embodiments, the solvents are removed using rotary evaporation. In some embodiments, the solvents are removed by lyophilization. In some embodiments, the second aqueous solution was filtered before removal of the solvents.

Removal of Water from the Second Aqueous Solution

In some embodiments, upon removal of the organic solvent from the second aqueous solution, the water can be removed from the second aqueous solution to provide a solid.

In some embodiments, the second aqueous solution is filtered before removal of water. For example, the second aqueous solution can be filtered by a 0.22 micron filter before removal of water.

As used herein, the term “micron” refers to a unit of measure of one one-thousandth of a millimeter.

In some embodiments, the water is removed under a vacuum. In some embodiments, the water is removed using rotary evaporation. In some embodiments, the water is removed by lyophilization.

### Reconstitution of the Solid

In some embodiments, the solid composition (e.g., the solid composition prepared by removing organic solvent from the second aqueous solution and removing water from the second aqueous solution) is mixed with water. In some embodiments, the solid composition (e.g., the solid composition prepared by removing organic solvent from the second aqueous solution and removing water from the second aqueous solution) is mixed with an aqueous solution. In some embodiments, the aqueous solution is a saline solution. In some embodiments, the aqueous solution is a 5% Dextrose water solution. In some embodiments, the mixing is the addition of water or the water solution to the solid. In some embodiments, the mixing is the addition of the solid to water or the water solution. In some embodiments, the mixing reconstitutes the solid. In some embodiments, the mixing yields a clear aqueous solution. In some embodiments, the range of pH in the reconstituted solution is from about 5 to about 8, from about 5 to about 7, from about 6 to about 7, from about 6.5 to about 7.5, from about 4 to about 6, or from about 6 to about 6.5. In some embodiments, the pH of the reconstituted solution is about 5, about 6, about 6.4, about 6.5, about 6.6, about 6.7, about 6.8, about 6.9, about 7, about 7.1, about 7.5, or about 8.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

### Composition Prepared by the Process

In some embodiments, the present disclosure provides a composition comprising docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate, produced by a method comprising the steps of:
(i) obtaining an organic solution of docetaxel in a polar water-miscible organic solvent:
(ii) obtaining a first aqueous solution of human serum albumin with sodium caprylate as stabilizer in the aqueous solution, which does not contain sodium N-acetyltryptophanate; and
(iii) mixing the organic solution of docetaxel and the first aqueous solution of human serum albumin to obtain a second aqueous solution comprising the composition comprising docetaxel, human serum albumin and sodium caprylate.

In some embodiments, at least 96% of the proteins in the composition is human serum albumin.

In some embodiments, the docetaxel and the human serum albumin in the composition are in a ratio by weight from about 1:70 to about 1:250. In some embodiments, the docetaxel and the human serum albumin in the composition are in a ratio by weight from about 1:80 to about 1:200. In some embodiments, the docetaxel and the human serum albumin in the composition are in a ratio by weight from about 1:90 to about 1:200. In some embodiments, the docetaxel and the human serum albumin in the composition are in a ratio by weight from about 1:95 to about 1:200. In some embodiments, the docetaxel and the human serum albumin in the composition are in a ratio by weight from about 1:100 to about 1:200. In some embodiments, the docetaxel and the human serum albumin in the composition are in a ratio by weight from about 1:80 to about 1:180. In some embodiments, the docetaxel and the human serum albumin in the composition are in a ratio by weight from about 1:90 to about 1:180. In some embodiments, the docetaxel and the human serum albumin in the composition are in a ratio by weight from about 1:100 to about 1:180. In some embodiments, the docetaxel and the human serum albumin in the composition are in a ratio by weight from about 1:80 to about 1:150. In some embodiments, the docetaxel and the human serum albumin in the composition are in a ratio by weight from about 1:90 to about 1:150. In some embodiments, the docetaxel and the human serum albumin in the composition are in a ratio by weight from about 1:95 to about 1:150. In some embodiments, the docetaxel and the human serum albumin in the composition are in a ratio by weight from about 1:100 to about 1:150. In some embodiments, the docetaxel and the human serum albumin in the composition are in a ratio by weight from about 1:80 to about 1:140. In some embodiments, the docetaxel and the human serum albumin in the composition are in a ratio by weight from about 1:90 to about 1:140. In some embodiments, the docetaxel and the human serum albumin in the composition are in a ratio by weight from about 1:95 to about 1:140. In some embodiments, the docetaxel and the human serum albumin in the composition are in a ratio by weight from about 1:100 to about 1:140. In some embodiments, the docetaxel and the human serum albumin in the composition are in a ratio by weight from about 1:80 to about 1:130. In some embodiments, the docetaxel and the human serum albumin in the composition are in a ratio by weight from about 1:90 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:100 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:95 to about 1:125. In some embodiments, the docetaxel and the human serum albumin in the composition are in a ratio by weight from about 1:100 to about 1:125. In some embodiments, the docetaxel and the human serum albumin in the composition are in a ratio by weight from about 1:90 to about 1:120. In some embodiments, the docetaxel and the human serum albumin in the composition are in a ratio by weight from about 1:100 to about 1:120. In some embodiments, the docetaxel and the human serum albumin in the composition are in a ratio by weight from about 1:90 to about 1:110. In some embodiments, the docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:170, about 1:180, about 1:190, about 1:200, about 1:210, about 1:220, or about 1:250.

In some embodiments, the human serum albumin is a native human serum albumin. In some embodiments, the human serum albumin is a native human serum albumin obtained from pools of human plasma. In some embodiments, the human serum albumin is a native human serum albumin obtained from pools of human plasma by low temperature controlled fractionation according to the Cohn process.

In some embodiments, the ratio of the amount of sodium caprylate to the amount of the proteins in the composition is from about 0.100 mmol to about 0.200 mmol of sodium caprylate per one gram of the proteins. In some embodiments, the ratio of the amount of sodium caprylate to the amount of the proteins in the composition is from about 0.120 mmol to about 0.190 mmol of sodium caprylate per one gram of the proteins. In some embodiments, the ratio of the amount of sodium caprylate to the amount of the proteins in the composition is from about 0.140 mmol to about 0.180 mmol of sodium caprylate per one gram of the proteins. In some embodiments, the ratio of the amount of sodium caprylate to the amount of the proteins in the composition is from about 0.145 mmol to about 0.175 mmol of sodium caprylate per one gram of the proteins. In some embodiments, the ratio of the amount of sodium caprylate to the amount of the proteins in the composition is from about 0.150 mmol to about 0.170 mmol of sodium caprylate per one gram of the proteins. In some embodiments, the ratio of the amount of sodium caprylate to the amount of the proteins in the composition is about 0.160 mmol of sodium caprylate per one gram of the proteins. In some embodiments, the ratio of the amount of sodium caprylate to the amount of the proteins in the composition is about 0.155 mmol of sodium caprylate per one gram of the proteins. In some embodiments, the ratio of the amount of sodium caprylate to the amount of the proteins in the composition is about 0.165 mmol of sodium caprylate per one gram of the proteins.

In some embodiments, the range of pH in the first aqueous solution is from about 5 to about 8, from about 5.5 to about 7.8, from about 6 to about 7.5, from about 6.5 to about 7, or from about 7 to about 7.5. In some embodiments, the pH of the first aqueous solution is about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, or about 8.

In some embodiments, the range of pH in the second aqueous solution is from about 5 to about 8, from about 5.5 to about 7.8, from about 6 to about 7.5, from about 6.5 to about 7, or from about 7 to about 7.5. In some embodiments, the pH of the first aqueous solution is about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, or about 8.

In some embodiments, the docetaxel can be a pharmaceutically acceptable salt of docetaxel. In some embodiments, the docetaxel can be a docetaxel with three equivalents of the water solvate. In some embodiments, docetaxel can be any one of crystal forms, amorphous forms, solvates and hydrates as described herein.

In some embodiments, the first aqueous solution of human serum albumin is an intravenous Human Albumin (human serum albumin) solution for infusion (e.g. Human Albumin; prepared as a 5%, 20%, or 25% protein solution) with sodium caprylate as stabilizer. In some embodiments, the first aqueous solution of human serum albumin is prepared from an intravenous Human Albumin (human serum albumin) solution for infusion (e.g. Human Albumin; prepared as a 5%, 20%, or 25% protein solution) with sodium caprylate as stabilizer. In some embodiments, the first aqueous solution of human serum albumin is prepared from an intravenous Human Albumin (human serum albumin) solution for infusion (e.g. Human Albumin; prepared as a 5%, 20%, or 25% protein solution) with sodium caprylate as stabilizer by diluted with water. Sodium caprylate is the only stabilizer used for the solutions of human serum albumin for infusion.

In some embodiments, the present disclosure provides a composition comprising docetaxel, proteins comprising a human serum albumin, sodium N-acetyltryptophanate, and sodium caprylate, wherein the docetaxel and the human serum albumin in the composition have a ratio by weight from about 1:80 to less than about 1:150, produced by a method comprising the steps of:
(i) obtaining an organic solution of docetaxel in a polar water-miscible organic solvent:
(ii) obtaining a first aqueous solution of human serum albumin with sodium N-acetyltryptophanate and sodium caprylate as stabilizers in the aqueous solution; and
(iii) mixing the organic solution of docetaxel and the first aqueous solution of human serum albumin to obtain a second aqueous solution comprising the composition comprising docetaxel, human serum albumin, sodium N-acetyltryptophanate and sodium caprylate.

In some embodiments, at least 96% of the proteins in the composition is human serum albumin.

In some embodiments, the docetaxel and the human serum albumin in the composition are in a ratio by weight from about 1:85 to less than 1:150. In some embodiments, the docetaxel and the human serum albumin in the composition are in a ratio by weight from about 1:90 to less than 1:150. In some embodiments, the docetaxel and the human serum albumin in the composition are in a ratio by weight from about 1:100 to less than 1:150. In some embodiments, the docetaxel and the human serum albumin in the composition are in a ratio by weight from about 1:95 to less than 1:150. In some embodiments, the docetaxel and the human serum albumin in the composition are in a ratio by weight from about 1:110 to less than 1:150. In some embodiments, the docetaxel and the human serum albumin in the composition are in a ratio by weight from about 1:80 to about 1:145. In some embodiments, the docetaxel and the human serum albumin in the composition are in a ratio by weight from about 1:90 to about 1:145. In some embodiments, the docetaxel and the human serum albumin in the composition are in a ratio by weight from about 1:100 to about 1:145. In some embodiments, the docetaxel and the human serum albumin in the composition are in a ratio by weight from about 1:95 to about 1:145. In some embodiments, the docetaxel and the human serum albumin in the composition are in a ratio by weight from about 1:110 to about 1:145. In some embodiments, the docetaxel and the human serum albumin in the composition are in a ratio by weight from about 1:80 to about 1:140. In some embodiments, the docetaxel and the human serum albumin in the composition are in a ratio by weight from about 1:90 to about 1:140. In some embodiments, the docetaxel and the human serum albumin in the composition are in a ratio by weight from about 1:100 to about 1:140. In some embodiments, the docetaxel and the human serum albumin in the composition are in a ratio by weight from about 1:95 to about 1:140. In some embodiments, the docetaxel and the human serum albumin in the composition are in a ratio by weight from about 1:110 to about 1:140. In some embodiments, the docetaxel and the human serum albumin in the composition are in a ratio by weight from about 1:80 to about 1:135. In some embodiments, the docetaxel and the human serum albumin in the composition are in a ratio by weight from about 1:90 to about 1:135. In some embodiments, the docetaxel and the human serum albumin in the composition are in a ratio by weight from about 1:95 to less than 1:135. In some embodiments, the docetaxel and the human serum albumin in the composition are in a ratio by weight from about 1:100 to about 1:135. In some embodiments, the docetaxel and the human serum albumin in the composition are in a ratio by weight from about 1:80 to about 1:130. In some embodiments, the docetaxel and the human serum albumin in the composition are in a ratio by weight from about 1:85 to about 1:130. In some embodiments, the docetaxel and the human serum albumin in the composition are in a ratio by weight from about 1:90 to about 1:130. In some embodiments, the docetaxel and the human serum albumin in the composition are in a ratio by weight from about 1:95 to about 1:130. In some embodiments, the docetaxel and the human serum albumin in the composition are in a ratio by weight from about 1:100 to about 1:130. In some embodiments, the docetaxel and the human serum albumin in the composition are in a ratio by weight from about 1:80 to about 1:125. In some embodiments, the docetaxel and the human serum albumin in the composition are in a ratio by weight from about 1:85 to about 1:125. In some embodiments, the docetaxel and the human serum albumin in the composition are in a ratio by weight from about 1:90 to about 1:125. In some embodiments, the docetaxel and the human serum albumin in the composition are in a ratio by weight from about 1:95 to about 1:125. In some embodiments, the docetaxel and the human serum albumin in the composition are in a ratio by weight from about 1:100 to about 1:125. In some embodiments, the docetaxel and the human serum albumin in the composition are in a ratio by weight from about 1:80 to about 1:120. In some embodiments, the docetaxel and the human serum albumin in the composition are in a ratio by weight from about 1:90 to about 1:120. In some embodiments, the docetaxel and the human serum albumin in the composition are in a ratio by weight from about 1:95 to about 1:120. In some embodiments, the docetaxel and the human serum albumin in the composition are in a ratio by weight from about 1:100 to about 1:120. In some embodiments, the docetaxel and the human serum albumin in the composition are in a ratio by weight from about 1:80 to about 1:110. In some embodiments, the docetaxel and the human serum albumin in the composition are in a ratio by weight from about 1:90 to about 1:110. In some embodiments, the docetaxel and the human serum albumin in the composition are in a ratio by weight from about 1:90 to about 1:115. In some embodiments, the docetaxel and the human serum albumin in the composition are in a ratio by weight from about 1:90 to about 1:100. In some embodiments, the docetaxel and the human serum albumin in the composition are in a ratio by weight from about 1:100 to about 1:110. In some embodiments, the docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, or about 1:145.

In some embodiments, the human serum albumin is a native human serum albumin. In some embodiments, the human serum albumin is a native human serum albumin obtained from pools of human plasma. In some embodiments, the human serum albumin is a native human serum albumin obtained from pools of human plasma by low temperature controlled fractionation according to the Cohn process.

In some embodiments, the composition comprises both sodium N-acetyltryptophanate and sodium caprylate. In some embodiments, the composition comprises both sodium N-acetyltryptophanate and sodium caprylate in a molar ratio of about 1:1. In some embodiments, the composition comprises from about 0.064 mmol to about 0.096 mmol sodium N-acetyltryptophanate and from about 0.064 mmol to about 0.096 mmol sodium caprylate per gram of the proteins in the composition. In some embodiments, the composition comprises from about 0.064 mmol to about 0.096 mmol sodium N-acetyltryptophanate per gram of the proteins in the composition. In some embodiments, the composition comprises from about 0.064 mmol to about 0.096 mmol sodium caprylate per gram of the proteins in the composition. In some embodiments, the composition comprises about 0.08 mmol sodium N-acetyltryptophanate and about 0.08 mmol sodium caprylate per gram of the proteins in the composition. In some embodiments, the composition comprises about 0.08 mmol sodium N-acetyltryptophanate per gram of the proteins in the composition. In some embodiments, the composition comprises about 0.08 mmol sodium caprylate per gram of the proteins in the composition.

In some embodiments, the range of pH in the first aqueous solution is from about 5 to about 8, from about 5.5 to about 7.8, from about 6 to about 7.5, from about 6.5 to about 7, or from about 7 to about 7.5. In some embodiments, the pH of the first aqueous solution is about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, or about 8.

In some embodiments, the range of pH in the second aqueous solution is from about 5 to about 8, from about 5.5 to about 7.8, from about 6 to about 7.5, from about 6.5 to about 7, or from about 7 to about 7.5. In some embodiments, the pH of the first aqueous solution is about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, or about 8.

In some embodiments, the docetaxel can be a pharmaceutically acceptable salt of docetaxel. In some embodiments, the docetaxel can be a docetaxel with three equivalents of the water solvate. In some embodiments, docetaxel can be any one of crystal forms, amorphous forms, solvates and hydrates as described herein.

In some embodiments, the first aqueous solution of human serum albumin is an intravenous Human Albumin (human serum albumin) solution for infusion (e.g. Human Albumin USP; prepared as a 5%, 20%, or 25% protein solution) with sodium N-acetyltryptophanate and sodium caprylate as stabilizers. In some embodiments, the first aqueous solution of human serum albumin is prepared from an intravenous Human Albumin (human serum albumin) solution for infusion (e.g. Human Albumin USP; prepared as a 5%, 20%, or 25% protein solution) with sodium N-acetyltryptophanate and sodium caprylate as stabilizers. In some embodiments, the first aqueous solution of human serum albumin is prepared from an intravenous Human Albumin (human serum albumin) solution for infusion (e.g. Human Albumin USP; prepared as a 5%, 20%, or 25% protein solution) with sodium N-acetyltryptophanate and sodium caprylate as stabilizers by diluted with water.

In some embodiments, the amount of the polar water-miscible organic solvent in the organic solution is from about 0.01 mL to about 5 mL per 1 mg of docetaxel.

In some embodiments, the amount of the polar water-miscible organic solvent in the organic solution is from about from about 0.1 mL to about 2.0 mL per 1 mg of docetaxel.

In some embodiments, the amount of aqueous solvent in the first aqueous solution is from about 0.005 mL to about 0.05 mL per 1 mg of human serum albumin.

In some embodiments, the amount of aqueous solvent in the first aqueous solution is from about 0.01 mL to about 0.05 mL per 1 mg of human serum albumin.

In some embodiments, the amount of aqueous solvent in the first aqueous solution is from about 0.015 mL to about 0.04 mL per 1 mg of human serum albumin.

In some embodiments, the polar water-miscible organic solvent is an alcohol selected from the group consisting of methanol, ethanol, isopropanol, n-butanol, and mixtures thereof.

In some embodiments, the polar water-miscible organic solvent is selected from methanol, ethanol, and mixtures thereof.

In some embodiments, the polar water-miscible organic solvent is methanol.

In some embodiments, the aqueous solvent is water.

In some embodiments, the mixing comprises adding the organic solution to the first aqueous solution. In some embodiments, wherein the mixing comprises adding the first aqueous solution to the organic solution. In some embodiments, the adding is carried out dropwise. In some embodiments, the adding is carried out for a period of time from several minutes to several hours. In some embodiments, the adding is carried out for a period of time from 2 min to 24 hours. In some embodiments, the adding is carried out for a period of time from 2 min minutes to 12 hours, from 2 min to 6 hours, from 3 min to 3 hours, from 2 min to 1 hour, from 2 min to 30 min, or from 2 min to 25 min.

In some embodiments, the mixing is carried out at a temperature from about 0° C. to about 25° C. In some embodiments, mixing is carried out at ambient temperature (e.g., about 25° C.). In some embodiments, the mixing is carried out at a temperature from about 0° C. to about 5° C. In some embodiments, the mixing is carried out at about 0° C.

In some embodiments, the volume ratio of the amount of aqueous solvent to the amount of the organic solvent in the second aqueous solution is in a range from about 1:1 to about 1000:1. In some embodiments, the volume ratio of the amount of aqueous solvent to the amount of the organic solvent in the second aqueous solution is in a range from about 1.5:1 to about 100:1. In some embodiments, the volume ratio of the amount of aqueous solvent to the amount of the organic solvent in the second aqueous solution is in a range from about 1.5:1 to about 20:1. In some embodiments, the volume ratio of the amount of aqueous solvent to the amount of the organic solvent in the second aqueous solution is in a range from about 1.5:1 to about 10:1. In some embodiments, the volume ratio of the amount of aqueous solvent to the amount of the organic solvent in the second aqueous solution is in a range from about 2:1 to about 10:1. In some embodiments, the volume ratio of the amount of aqueous solvent to the amount of the organic solvent in the second aqueous solution is about 1.5:1, about 2:1, about 2.2:1, about 2.3:1, about 2.4:1, about 2.5:1, about 3:1, about 4:1, about 5:1, about 6:1, about 7:1, about 8:1, about 9:1, or about 10:1. In some embodiments, the aqueous solvent is water. In some embodiments, the aqueous solvent is water and the organic solvent is an alcohol. In some embodiments, the aqueous solvent is water and the organic solvent is methanol.

In some embodiments, the methods further comprise the step of removing the polar water-miscible organic solvent from the second aqueous solution to obtain a third aqueous solution comprising the composition comprising docetaxel and human serum albumin. In some embodiments, the methods further comprise the step of removing aqueous solvent from the third aqueous solution to obtain the composition comprising docetaxel and human serum albumin.

In some embodiments, the methods further comprise the step(s) of removing the organic solvent (e.g. methanol) and the aqueous solvent (e.g., water) from the second aqueous solution to obtain the composition comprising docetaxel, proteins comprising a human serum albumin, and sodium caprylate.

In some embodiments, the methods further comprise the step(s) of removing the organic solvent (e.g. methanol) and the aqueous solvent (e.g., water) from the second aqueous solution to obtain the composition comprising docetaxel, proteins comprising a human serum albumin, sodium N-acetyltryptophanate and sodium caprylate.

In some embodiments, the removing is carried out in vacuum (e.g., using the rotovap). In some embodiments, the removing is carried out by lyophilization.

In some embodiments, the composition is a solid formulation.

In some embodiments, the composition is an aqueous formulation. In some embodiments, the aqueous formulation is substantially free of solvent other than water. In some embodiments, the aqueous formulation is free of a surfactant, which is selected from the group consisting of CREMOPHOR® surfactants and Polysorbate 80. In some embodiments, the aqueous formulation is a clear aqueous solution. In some embodiments, the aqueous formulation is a clear aqueous solution for at least 1 hours, at least 2 hours, at least 3 hours, at least 4 hours, at least 5 hours, at least 6 hours, at least 8 hours, or at least 24 hours.

In some embodiments, the concentration of docetaxel in the aqueous formulation is from about 0.1 mg per 1 ml to about 1 mg per 1 ml of the aqueous solvent. In some embodiments, the concentration of docetaxel in the aqueous formulation is from about 0.3 mg per 1 ml to about 0.75 mg per 1 ml of the aqueous solvent. In some embodiments, the concentration of docetaxel in the aqueous formulation is from about 0.3 mg per 1 ml to about 0.5 mg per 1 ml of the aqueous solvent. In some embodiments, the concentration of docetaxel in the aqueous formulation is from about 0.3 mg per 1 ml to about 0.4 mg per 1 ml of the aqueous solvent. In some embodiments, the concentration of docetaxel in the aqueous formulation is about 0.3 mg per 1 ml of the aqueous solvent. In some embodiments, the concentration of docetaxel in the aqueous formulation is about 0.33 mg per 1 ml of the aqueous solvent. In some embodiments, the concentration of docetaxel in the aqueous formulation is about 0.4 mg per 1 ml of the aqueous solvent. In some embodiments, the concentration of docetaxel in the aqueous formulation is about 0.5 mg per 1 ml of the aqueous solvent.

In some embodiments, the aqueous formulation is a clear aqueous solution, wherein the aqueous formulation is substantially free of solvent other than water, and wherein the concentration of docetaxel in the aqueous formulation is from about 0.1 mg per 1 ml to about 1 mg per 1 ml of the aqueous solvent. In some embodiments, the aqueous formulation is a clear aqueous solution, wherein the aqueous formulation is substantially free of solvent other than water, and wherein the concentration of docetaxel in the aqueous formulation is from about 0.3 mg per 1 ml to about 0.75 mg per 1 ml of the aqueous solvent. In some embodiments, the aqueous formulation is a clear aqueous solution, wherein the aqueous formulation is substantially free of solvent other than water, and wherein the concentration of docetaxel in the aqueous formulation is from about 0.25 mg per 1 ml to about 0.45 mg per 1 ml of the aqueous solvent. In some embodiments, the aqueous formulation is a clear aqueous solution, wherein the aqueous formulation is substantially free of solvent other than water, and wherein the concentration of docetaxel in the aqueous formulation is from about 0.3 mg per 1 ml to about 0.4 mg per 1 ml of the aqueous solvent. In some embodiments, the aqueous formulation is a clear aqueous solution, wherein the aqueous formulation is substantially free of solvent other than water, and wherein the concentration of docetaxel in the aqueous formulation is about 0.25 mg per 1 ml, about 0.3 mg per 1 ml, about 0.33 mg per 1 ml, about 0.4 mg per 1 ml, or about 0.5 mg per 1 ml of the aqueous solvent.

In some embodiments, the present disclosure provides a pharmaceutical composition comprising the composition as prepared by a process as described herein, and a pharmaceutically acceptable carrier.

In some embodiments, the present disclosure provides a method of treating a cancer, the method comprising the step of administering to a subject in need thereof a therapeutically effective amount of the pharmaceutical composition as described herein.

In some embodiments, the cancer is a solid tumor. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma.

### EXAMPLES

### Materials and Methods

HPLC analysis: The HPLC system used herein is a SHIMADZU LC-10AT vp series system, which consists of a SHIMADZU LC-10AT vp pump, a manual injector, a SHIMADZU CTO-10AS vp column oven, a SHIMADZU SPD-10A vp wavelength detector, and a SHIMADZU LC solution workstation. Waters XTERRA RP10 column (4.6 mm×150 mm, 5 μm) is used as an analytical HPLC column. The column oven temperature is 30° C. Mobile phase is composed of methanol and water (70:30, v/v) and pumped at a flow rate of 1 ml/minute. The effluent is detected at a wavelength of 233 nm using a UV detector. The sample injection amount is 20 μl.

### Example 1: Composition Comprising Docetaxel and Human Serum Albumin (HSA)

The ratio by weight of docetaxel to HSA prepared was about 1:110, about 1:120, or about 1:150.

Docetaxel (2 mg) was dissolved in methanol (1.3 ml) in a glass vial to give a clear solution. A solution of HSA (220 mg, 1.1 ml) (20% Human Albumin solution for infusion with sodium caprylate (0.140˜0.180 mmol per gram of proteins) as the only stabilizer in the solution) was added into 1.9 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 7 hours at room temperature (about 20˜25° C.).

Docetaxel (2 mg) was dissolved in methanol (1.3 ml) in a glass vial to give a clear solution. A solution of HSA (240 mg, 1.2 ml) (20% Human Albumin solution for infusion with sodium caprylate (0.140˜0.180 mmol per gram of proteins) as the only stabilizer in the solution) was added into 1.8 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 7 hours at room temperature (about 20˜25° C.). After 24 hours at room temperature (about 20˜25)° C., white precipitation was formed in the aqueous solution.

Docetaxel (2 mg) was dissolved in methanol (1.3 ml) in a glass vial to give a clear solution. A solution of HSA (300 mg, 1.5 ml) (20% Human Albumin solution for infusion with sodium caprylate (0.140˜0.180 mmol per gram of proteins) as the only stabilizer in the solution) was added into 1.5 ml of water to give a HSA solution (3 ml) in a round bottom flask. The methanol solution of docetaxel was added slowly dropwise into the flask of the HSA solution with rapid stirring at (° C.) Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 7 hours at room temperature (about 20˜25° C.). After 24 hours at room temperature (about 20˜25° C.), white precipitation was formed in the aqueous solution.

### Example 2: Composition Comprising Docetaxel and Human Serum Albumin (HSA)

The ratio by weight of docetaxel to HSA prepared was about 1:120 or about 1:150.

Docetaxel (2 mg) was dissolved in methanol (1.3 ml) in a glass vial to give a clear solution. A solution of HSA (240 mg, 1.2 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) was added into 1.8 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 3 hours at room temperature (about 20˜25° C.). After 4 hours at room temperature (about 20˜25° C.), white precipitation was formed in the aqueous solution.

Docetaxel (2 mg) was dissolved in methanol (1.3 ml) in a glass vial to give a clear solution. A solution of HSA (300 mg, 1.5 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) was added into 1.5 ml of water to give a HSA solution (3 ml) in a round bottom flask. The methanol solution of docetaxel was added slowly dropwise into the flask of the HSA solution with rapid stirring at (° C.) Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 6 hours at room temperature (about 20˜25° C.). After 7 hours at room temperature (about 20˜25° C.), white precipitation was formed in the aqueous solution.

### Example 3: Composition Comprising Docetaxel and Human Serum Albumin (HSA)

The ratio by weight of docetaxel to HSA prepared was about 1:120.

Docetaxel (15 mg) was dissolved in methanol (7.7 ml) in a glass vial to give a clear solution. A solution of HSA (1800 mg, 9 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) was added into 9 ml of water to give a HSA solution (18 ml) in a round bottom flask. The methanol solution of docetaxel was added slowly dropwise into the flask of the HSA solution with rapid stirring at (° C.) Upon completion of the addition, a clear solution was obtained. Then, the methanol in the solution was removed under vacuum to give a clear solution. The clear aqueous solution was filtered by a 0.22 micron aqueous phase filter. The resulting aqueous solution was lyophilized overnight to give a white solid. A sample of 50 mg of the lyophilized solid was reconstituted by adding 1 mL water to give a clear solution. The clear aqueous solution stayed clear without any solid precipitation up to 6 hours at room temperature (about 20˜25° C.). After 7 hours at room temperature (about 20˜25° C.), white precipitation was formed in the aqueous solution. Another sample of 50 mg of the lyophilized solid was reconstituted by adding 1 mL water to give a clear solution. The clear aqueous solution stayed clear without any solid precipitation up to 24 hours in refrigerator (about 2˜5° C.).

Docetaxel (15 mg) was dissolved in methanol (7.7 ml) in a glass vial to give a clear solution. A solution of HSA (1800 mg, 9 ml) (20% Human Albumin solution for infusion with sodium caprylate (0.140˜0.180 mmol per gram of proteins) as the only stabilizer in the solution) was added into 9 ml of water to give a HSA solution (18 ml) in a round bottom flask. The methanol solution of docetaxel was added slowly dropwise into the flask of the HSA solution with rapid stirring at 0° C. Upon completion of the addition, a clear solution 10 was obtained. Then, the methanol in the solution was removed under vacuum to give a clear solution. The clear aqueous solution was filtered by a 0.22 micron aqueous phase filter. The resulting aqueous solution was lyophilized overnight to give a white solid. A sample of 50 mg of the lyophilized solid was reconstituted by adding 1 mL water to give a clear solution. The clear aqueous solution stayed clear without any solid precipitation up to 7 hours at room temperature (about 20˜25° C.). After 8 hours at room temperature (about 20˜25° C.), white precipitation was formed in the aqueous solution. Another sample of 50 mg of the lyophilized solid was reconstituted by adding 1 mL water to give a clear solution. The clear aqueous solution stayed clear without any solid precipitation up to 24 hours in refrigerator (about 2˜5° C.).

### Example 4: Composition Comprising Docetaxel and Human Serum Albumin (HSA)

The ratio by weight of docetaxel to HSA prepared was about 1:120, about 1:130 about 140, or about 1:150.

Docetaxel (2 mg) was dissolved in methanol (1.3 ml) in a glass vial to give a clear solution. A solution of HSA (240 mg, 1.2 ml) (20% Human Albumin solution for infusion with sodium caprylate (0.140˜0.180 mmol per gram of proteins) as the only stabilizer in the solution) was added into 1.8 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 4 hours at room temperature (about 20˜25° C.). After 5 hours at room temperature (about 20˜25° C.), white precipitation was formed in the aqueous solution. Another sample of 100 mg of the lyophilized solid was reconstituted by adding 2 mL water to give a clear solution. The clear aqueous solution stayed clear without any solid precipitation up to 3 days in refrigerator (about 2˜5° C.).

Docetaxel (2 mg) was dissolved in methanol (1.3 ml) in a glass vial to give a clear solution. A solution of HSA (260 mg, 1.3 ml) (20% Human Albumin solution for infusion with sodium caprylate (0.140˜0.180 mmol per gram of proteins) as the only stabilizer in the solution) was added into 1.7 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 7 hours at room temperature (about 20˜25° C.). After 8 hours at room temperature (about 20˜25° C.), white precipitation was formed in the aqueous solution. Another sample of 100 mg of the lyophilized solid was reconstituted by adding 2 mL water to give a clear solution. The clear aqueous solution stayed clear without any solid precipitation up to 3 days in refrigerator (about 2˜5° C.).

Docetaxel (2 mg) was dissolved in methanol (1.3 ml) in a glass vial to give a clear solution. A solution of HSA (280 mg, 1.4 ml) (20% Human Albumin solution for infusion with sodium caprylate (0.140˜0.180 mmol per gram of proteins) as the only stabilizer in the solution) was added into 1.6 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 10 hours at room temperature (about 20˜25° C.). After 11 hours at room temperature (about 20˜25° C.), white precipitation was formed in the aqueous solution. Another sample of 100 mg of the lyophilized solid was reconstituted by adding 2 mL water to give a clear solution. The clear aqueous solution stayed clear without any solid precipitation up to 3 days in refrigerator (about 2˜5° C.).

Docetaxel (2 mg) was dissolved in methanol (1.3 ml) in a glass vial to give a clear solution. A solution of HSA (300 mg, 1.5 ml) (20% Human Albumin solution for infusion with sodium caprylate (0.140˜0.180 mmol per gram of proteins) as the only stabilizer in the solution) was added into 1.5 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 100 mg of the lyophilized solid was reconstituted by adding 2 mL water to give a clear solution. The clear aqueous solution stayed clear without any solid precipitation up to 9 hours at room temperature (about 20˜25° C.). After 9 hours at room temperature (about 20˜25° C.), white precipitation was formed in the aqueous solution. Another sample of 100 mg of the lyophilized solid was reconstituted by adding 2 mL water to give a clear solution. The clear aqueous solution stayed clear without any solid precipitation up to 3 days in refrigerator (about 2˜5° C.).

### Example 5: Composition Comprising Docetaxel and Human Serum Albumin (HSA)

The ratio by weight of docetaxel to HSA prepared was about 1:120.

Docetaxel (30 mg) was dissolved in methanol (15.4 ml) in a glass vial to give a clear solution. A solution of HSA (3600 mg, 18 ml) (20% Human Albumin solution for infusion with sodium caprylate (0.140˜0.180 mmol per gram of proteins) as the only stabilizer in the solution) was added into 18 ml of water to give a HSA solution (36 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting aqueous solution was lyophilized overnight to give a white solid.

A sample of 100 mg of the lyophilized solid was reconstituted by adding 2 mL water to give a clear solution. The clear aqueous solution stayed clear without any solid precipitation up to 9 hours at room temperature (about 20˜25° C.).

A sample of 500 mg of the lyophilized solid was reconstituted by adding 10 mL water to give a clear solution. The clear aqueous solution stayed clear without any solid precipitation up to 9 hours at room temperature (about 20˜25° C.).

A sample of 180 mg of the lyophilized solid was reconstituted by adding 2 mL water to give a cloudy solution with white precipitation. A sample of 140 mg of the lyophilized solid was reconstituted by adding 2 mL water to give a cloudy solution with white precipitation.

### Example 6: Composition Comprising Docetaxel and Human Serum Albumin (HSA)

The ratio by weight of docetaxel to HSA prepared was about 1:90, about 1:100, about 110, or about 1:120.

Docetaxel (8 mg) was dissolved in methanol (3.1 ml) in a glass vial to give a clear solution. A solution of HSA (720 mg, 3.6 ml) (20% Human Albumin solution for infusion with sodium caprylate (0.140˜0.180 mmol per gram of proteins) as the only stabilizer in the solution) was added into 3.6 ml of water to give a HSA solution (7.2 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 70 mg of the lyophilized solid was reconstituted by adding 2 mL water to give a clear solution. The clear aqueous solution stayed clear without any solid precipitation up to 4 hours at room temperature (about 20˜25° C.). After 5 hours at room temperature (about 20˜25° C.), white precipitation was formed in the aqueous solution. A sample of 80 mg of the lyophilized solid was reconstituted by adding 2 mL water to give a clear solution. The clear aqueous solution stayed clear without any solid precipitation up to 4 hours at room temperature (about 20˜25° C.). After 5 hours at room temperature (about 20˜25° C.), white precipitation was formed in the aqueous solution. A sample of 100 mg of the lyophilized solid was reconstituted by adding 2 mL water to give a cloudy solution with white precipitation. A sample of 120 mg of the lyophilized solid was reconstituted by adding 2 mL water to give a cloudy solution with white precipitation.

Docetaxel (8 mg) was dissolved in methanol (3.4 ml) in a glass vial to give a clear solution. A solution of HSA (800 mg, 4.0 ml) (20% Human Albumin solution for infusion with sodium caprylate (0.140˜0.180 mmol per gram of proteins) as the only stabilizer in the solution) was added into 4.0 ml of water to give a HSA solution (8 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 70 mg of the lyophilized solid was reconstituted by adding 2 mL water to give a clear solution. The clear aqueous solution stayed clear without any solid precipitation up to 4 hours at room temperature (about 20˜25° C.). After 5 hours at room temperature (about 20˜25° C.), white precipitation was formed in the aqueous solution. A sample of 80 mg of the lyophilized solid was reconstituted by adding 2 mL water to give a clear solution. The clear aqueous solution stayed clear without any solid precipitation up to 4 hours at room temperature (about 20˜25° C.). After 5 hours at room temperature (about 20˜25° C.), white precipitation was formed in the aqueous solution. A sample of 100 mg of the lyophilized solid was reconstituted by adding 2 mL water to give a clear solution. The clear aqueous solution stayed clear without any solid precipitation up to 5 hours at room temperature (about 20˜25° C.). After 6 hours at room temperature (about 20˜25° C.), white precipitation was formed in the aqueous solution. A sample of 120 mg of the lyophilized solid was reconstituted by adding 2 mL water to give a cloudy solution with white precipitation.

Docetaxel (8 mg) was dissolved in methanol (3.8 ml) in a glass vial to give a clear solution. A solution of HSA (880 mg, 4.4 ml) (20% Human Albumin solution for infusion with sodium caprylate (0.140˜0.180 mmol per gram of proteins) as the only stabilizer in the solution) was added into 4.4 ml of water to give a HSA solution (8.8 ml) in a round bottom flask. The methanol solution of docetaxel was added slowly dropwise into the flask of the HSA solution with rapid stirring at 0) ° C. Upon completion of the addition, a clear solution was obtained. Then, the methanol in the solution was removed under vacuum to give a clear solution. The resulting clear aqueous solution was lyophilized overnight to give a white solid. A sample of 70 mg of the lyophilized solid was reconstituted by adding 2 mL water to give a clear solution. The clear aqueous solution stayed clear without any solid precipitation up to 4 hours at room temperature (about 20˜25)° C. After 5 hours at room temperature (about 20˜25° C.), white precipitation was formed in the aqueous solution. A sample of 80 mg of the lyophilized solid was reconstituted by adding 2 mL water to give a clear solution. The clear aqueous solution stayed clear without any solid precipitation up to 4 hours at room temperature (about 20˜25)° C. After 5 hours at room temperature (about 20˜25° C.), white precipitation was formed in the aqueous solution. A sample of 100 mg of the lyophilized solid was reconstituted by adding 2 mL water to give a clear solution. The clear aqueous solution stayed clear without any solid precipitation up to 4 hours at room temperature (about 20˜25° C.). After 5 hours at room temperature (about 20˜25° C.), white precipitation was formed in the aqueous solution. A sample of 120 mg of the lyophilized solid was reconstituted by adding 2 mL water to give a cloudy solution with white precipitation.

Docetaxel (8 mg) was dissolved in methanol (4.1 ml) in a glass vial to give a clear solution. A solution of HSA (960 mg, 4.8 ml) (20% Human Albumin solution for infusion with sodium caprylate (0.140˜0.180 mmol per gram of proteins) as the only stabilizer in the solution) was added into 4.8 ml of water to give a HSA solution (9.6 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid. A sample of 70 mg of the lyophilized solid was reconstituted by adding 2 mL water to give a clear solution. The clear aqueous solution stayed clear without any solid precipitation up to 5 hours at room temperature (about 20˜25° C.). After 24 hours at room temperature (about 20˜25° C.), white precipitation was formed in the aqueous solution. A sample of 80 mg of the lyophilized solid was reconstituted by adding 2 mL water to give a clear solution. The clear aqueous solution stayed clear without any solid precipitation up to 5 hours at room temperature (about 20˜25° C.). After 24 hours at room temperature (about 20˜25° C.), white precipitation was formed in the aqueous solution. A sample of 100 mg of the lyophilized solid was reconstituted by adding 2 mL water to give a clear solution. The clear aqueous solution stayed clear without any solid precipitation up to 5 hours at room temperature (about 20˜25)° C. After 24 hours at room temperature (about 20˜25° C.), white precipitation was formed in the aqueous solution. A sample of 120 mg of the lyophilized solid was reconstituted by adding 2 mL water to give a clear solution. The clear aqueous solution stayed clear without any solid precipitation up to 5 hours at room temperature (about 20˜25° C.). After 24 hours at room temperature (about 20˜25° C.), white precipitation was formed in the aqueous solution.

### Example 7: Composition Comprising Docetaxel and Human Serum Albumin (HSA)

The ratio by weight of docetaxel to HSA prepared was about 1:120.

Docetaxel (3 mg) was dissolved in methanol (1.3 ml) in a glass vial to give a clear solution. A solution of HSA (360 mg, 1.8 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) was added into 1.2 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid, which was reconstituted by adding 7.5 mL water to give a clear solution. The clear aqueous solution stayed clear without any solid precipitation up to 7 hours at room temperature (about 20˜25)° C. After 8 hours at room temperature (about 20˜25° C.), white precipitation was formed in the aqueous solution.

The above procedure was repeated using Docetaxel (3 mg) and a solution of HSA (360 mg, 1.8 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) to give a white solid, which was reconstituted by adding 9 mL water to give a clear solution. The clear aqueous solution stayed clear without any solid precipitation up to 7 hours at room temperature (about 20˜25° C.). After 8 hours at room temperature (about 20˜25° C.), white precipitation was formed in the aqueous solution.

Docetaxel (3 mg) was dissolved in methanol (1.3 ml) in a glass vial to give a clear solution. A solution of HSA (360 mg, 1.8 ml) (20% Human Albumin solution for infusion with sodium caprylate (0.140˜0.180 mmol per gram of proteins) as the only stabilizer in the solution) was added into 1.2 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid, which was reconstituted by adding 7.5 mL water to give a clear solution. The clear aqueous solution stayed clear without any solid precipitation up to 7 hours at room temperature (about 20˜25)° C. After 8 hours at room temperature (about 20˜25° C.), white precipitation was formed in the aqueous solution.

The above procedure was repeated using Docetaxel (3 mg) and a solution of HSA (360 mg, 1.8 ml) (20% Human Albumin solution for infusion with sodium caprylate (0.140˜0.180 mmol per gram of proteins) as the only stabilizer in the solution) to give a white solid, which was reconstituted by adding 9 mL water to give a clear solution. The clear aqueous solution stayed clear without any solid precipitation up to 6 hours at room temperature (about 20˜25° C.). After 7 hours at room temperature (about 20˜25° C.), white precipitation was formed in the aqueous solution.

### Example 8: Composition Comprising Docetaxel and Human Serum Albumin (HSA)

The ratio by weight of docetaxel to HSA prepared was about 1:100.

Docetaxel (3 mg) was dissolved in methanol (1.3 ml) in a glass vial to give a clear solution. A solution of HSA (300 mg, 1.5 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) was added into 1.5 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid, which was reconstituted by adding 9 mL water to give a clear solution. The clear aqueous solution stayed clear without any solid precipitation up to 9 hours at room temperature (about 20˜25° C.).

The above procedure was repeated using Docetaxel (3 mg) and a solution of HSA (300 mg, 1.5 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) to give a white solid, which was reconstituted by adding 10 mL water to give a clear solution. The clear aqueous solution stayed clear without any solid precipitation up to 9 hours at room temperature (about 20˜25° C.).

Docetaxel (3 mg) was dissolved in methanol (1.3 ml) in a glass vial to give a clear solution. A solution of HSA (300 mg, 1.5 ml) (20% Human Albumin solution for infusion with sodium caprylate (0.140˜0.180 mmol per gram of proteins) as the only stabilizer in the solution) was added into 1.5 ml of water to give a HSA solution (3 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid, which was reconstituted by adding 9 mL water to give a clear solution. The clear aqueous solution stayed clear without any solid precipitation up to 9 hours at room temperature (about 20˜25° C.).

The above procedure was repeated using Docetaxel (3 mg) and a solution of HSA (300 mg, 1.5 ml) (20% Human Albumin solution for infusion with sodium caprylate (0.140˜0.180 mmol per gram of proteins) as the only stabilizer in the solution) to give a white solid, which was reconstituted by adding 10 mL water to give a clear solution. The clear aqueous solution stayed clear without any solid precipitation up to 9 hours at room temperature (about 20˜25° C.).

### Example 9: Measure pH Value of the Clear Aqueous Solution of Composition Comprising Docetaxel and Human Serum Albumin (HSA)

500 mg of the lyophilized solid of the composition comprising docetaxel and HSA (the ratio by weight about 1:120) from Example 5 was dissolved in 10 ml of water to give a clear aqueous solution. The clear aqueous solution was kept at about 25° C., and measured for pH value. The pH value of the clear aqueous solution is 7.10 (3 measurements: 7.10, 7.10, and 7.09).

400 mg of the lyophilized solid of the composition comprising docetaxel and HSA (the ratio by weight about 1:120) from Example 5 was dissolved in 10 ml of water to give a clear aqueous solution. The clear aqueous solution was kept at about 25° C., and measured for pH value. The pH value of the clear aqueous solution is 7.08 (3 measurements: 7.07, 7.08, and 7.08).

300 mg of the lyophilized solid of the composition comprising docetaxel and HSA (the ratio by weight about 1:120) from Example 5 was dissolved in 10 ml of water to give a clear aqueous solution. The clear aqueous solution was kept at about 25° C., and measured for pH value. The pH value of the clear aqueous solution is 7.08 (3 measurements: 7.07, 7.08, and 7.08).

500 mg of the lyophilized solid of the composition comprising docetaxel and HSA (the ratio by weight about 1:120) from Example 5 was dissolved in 10 ml of 0.9% saline solution, which had pH value about 5.41, to give a clear aqueous solution. The clear aqueous solution was kept at about 25° C., and measured for pH value. The pH value of the clear aqueous solution is 6.94 (3 measurements: 6.94, 6.95, and 6.94).

400 mg of the lyophilized solid of the composition comprising docetaxel and HSA (the ratio by weight about 1:120) from Example 5 was dissolved in 10 ml of 0.9% saline solution, which had pH value about 5.41, to give a clear aqueous solution. The clear aqueous solution was kept at about 25° C., and measured for pH value. The pH value of the clear aqueous solution is 6.94 (3 measurements: 6.94, 6.94, and 6.94).

300 mg of the lyophilized solid of the composition comprising docetaxel and HSA (the ratio by weight about 1:120) from Example 5 was dissolved in 10 ml of 0.9% saline solution, which had pH value about 5.41, to give a clear aqueous solution. The clear aqueous solution was kept at about 25° C., and measured for pH value. The pH value of the clear aqueous solution is 6.92 (3 measurements: 6.92, 6.92, and 6.93).

### Example 10: Composition Comprising Docetaxel and Human Serum Albumin (HSA)

The ratio by weight of docetaxel to HSA prepared was about 1:110.

Docetaxel (16 mg) was dissolved in methanol (7.5 ml) in a glass vial to give a clear solution. A solution of HSA (1760 mg, 8.8 ml) (20% Human Albumin solution for infusion with sodium caprylate (0.140˜0.180 mmol per gram of proteins) as the only stabilizer in the solution) was added into 8.8 ml of water to give a HSA solution (17.6 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting aqueous solution was lyophilized overnight to give a white solid.

### Example 11: Composition Comprising Docetaxel and Human Serum Albumin (HSA)

The ratio by weight of docetaxel to HSA prepared was about 1:100.

Docetaxel (30 mg) was dissolved in methanol (12.9 ml) in a glass vial to give a clear solution. A solution of HSA (3000 mg, 15 ml) (20% Human Albumin solution for infusion with sodium caprylate (0.140˜0.180 mmol per gram of proteins) as the only stabilizer in the solution) was added into 15 ml of water to give a HSA solution (30 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting aqueous solution was lyophilized overnight to give a white solid.

### Example 12: Composition Comprising Docetaxel and Human Serum Albumin (HSA)

The ratio by weight of docetaxel to HSA prepared was about 1:100.

Docetaxel (15 mg) was dissolved in methanol (6.4 ml) in a glass vial to give a clear solution. A solution of HSA (1500 mg, 7.5 ml) (20% Human Albumin solution for infusion (20% Human Albumin UPS) with sodium caprylate (about 0.08 mmol per gram of proteins) and sodium N-acetyltryptophanate (about 0.08 mmol per gram of proteins) as the stabilizers in the solution) was added into 7.5 ml of water to give a HSA solution (15 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting aqueous solution was lyophilized overnight to give a white solid.

### Example 13: Composition Comprising Docetaxel and Human Serum Albumin (HSA)

The ratio by weight of docetaxel to HSA prepared was about 1:100.

Docetaxel (15 mg) was dissolved in methanol (6.4 ml) in a glass vial to give a clear solution. A solution of HSA (1500 mg, 7.5 ml) (20% Human Albumin solution for infusion with sodium caprylate (0.140˜0.180 mmol per gram of proteins) as the only stabilizer in the solution) was added into 7.5 ml of water to give a HSA solution (15 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting aqueous solution was lyophilized overnight to give a white solid.

### Example 14: Measure the Correlation Between HPLC Peak Area and the Docetaxel Concentration

Methanol solutions of docetaxel in 6 different concentrations, 0.0125 mg/ml, 0.025 mg/ml, 0.05 mg/ml, 0.075 mg/ml, 0.1 mg/ml, and 0.125 mg/ml, were prepared. The 6 docetaxel methanol solutions were analyzed in HPLC. The peak area and concentration of docetaxel were correlated using linear regression. The linear regression data is shown as below.

Y (peak area)=−8459+2.798E7*X (concentration), R=0.9999, P<0.0001.

### Example 15: Measure the Docetaxel Concentrations in the Clear Aqueous Solutions Before the Filtration at 0 Hour, and after the Filtration at 1 Hour, 2 Hour, 3 Hour, 4 Hour, and 5 Hour

1.6 g of the lyophilized solid of the composition comprising docetaxel and HSA (the ratio by weight about 1:110) from Example 10 was dissolved in 32 ml of water to give a clear aqueous solution in a flask, which was kept at about 20˜25° C. Immediately after the lyophilized solid was dissolved in water, 1 ml of the clear aqueous solution was taken out from the 32 ml solution. Then 1 ml of the solution was named as the solution DC-0-0h. To 200 μl of the solution DC-0-0h was added 800 μl of acetonitrile. The mixture was vortexed for seconds and then centrifuged at 4,000 g for 5 minutes. The supernatants were removed and collected followed by injection on HPLC. The same procedure was repeated 2 more times for the solution DC-0-0h. Based on the HPLC data and the measurement data of Example 14, the docetaxel concentrations of the supernatants prepared from solution of DC-0-0h have been calculated and shown in the Table 1.

TABLE 1

| | Docetaxel | Average Docetaxel |
|---|---|---|
| Solution Number | Concentration (mg/ml) | Concentration (mg/ml) |
| DC-0-0h-1 | 0.08640 | 0.08608 |
| DC-0-0h-2 | 0.08596 | |
| DC-0-0h-3 | 0.08587 | |

At 1 hour, additional 5 ml of the clear aqueous solution was taken out from the remaining clear aqueous solution in the flask. Then 1 ml of the solution was taken out from the 5 ml clear aqueous solution and filtered by a 0.22 micron aqueous phase filter to give the solution DC-1-1h, and the remaining 4 ml of the solution was filtered by the same 0.22 micron aqueous phase filter at 1 ml at a time to give the solutions DC-2-1h, DC-3-1h, DC-4-1h, and DC-5-1h. To 200 μl of the solution DC-5-1h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution DC-5-1h. Based on the HPLC data and the measurement data of Example 14, the docetaxel concentrations of the supernatants prepared from solution of DC-5-1h have been calculated and shown in the Table 2. At 1 hour, the docetaxel concentration of the clear aqueous solution after the filtration was about 96.56% of the docetaxel concentration of the clear aqueous solution at 0 hour before the filtration.

TABLE 2

| | Docetaxel | Average Docetaxel |
|---|---|---|
| Solution Number | Concentration (mg/ml) | Concentration (mg/ml) |
| DC-5-1h-1 | 0.08352 | 0.08312 |
| DC-5-1h-2 | 0.08284 | |
| DC-5-1h-3 | 0.08300 | |

At 2 hour, 5 ml of the clear aqueous solution was taken out from the remaining clear aqueous solution in the flask. The experiments were done for the 5 ml of the clear aqueous solution taken out at 2 hour using the same protocol as for the 5 ml of the clear aqueous solution taken out at 1 hour. Based on the HPLC data and the measurement data of Example 14, the docetaxel concentrations of the supernatants prepared from solution of DC-5-2h have been calculated and shown in the Table 3. At 2 hour, the docetaxel concentration of the clear aqueous solution after the filtration was about 97.01% of the docetaxel concentration of the clear aqueous solution at 0 hour before the filtration.

TABLE 3

| | Docetaxel | Average Docetaxel |
|---|---|---|
| Solution Number | Concentration (mg/ml) | Concentration (mg/ml) |
| DC-5-2h-1 | 0.08351 | 0.08351 |
| DC-5-2h-2 | 0.08347 | |
| DC-5-2h-3 | 0.08354 | |

At 3 hours, 5 ml of the clear aqueous solution was taken out from the remaining clear aqueous solution in the flask. The experiments were done for the 5 ml of the clear aqueous solution taken out at 3 hour using the same protocol as for the 5 ml of the clear aqueous solution taken out at 1 hour. Based on the HPLC data and the measurement data of Example 14, the docetaxel concentrations of the supernatants prepared from solution of DC-5-3h have been calculated and shown in the Table 4. At 3 hours, the docetaxel concentration of the clear aqueous solution after the filtration was about 97.32% of the docetaxel concentration of the clear aqueous solution at 0 hour before the filtration.

TABLE 4

| | Docetaxel | Average Docetaxel |
|---|---|---|
| Solution Number | Concentration (mg/ml) | Concentration (mg/ml) |
| DC-5-3h-1 | 0.08407 | 0.08377 |
| DC-5-3h-2 | 0.08364 | |
| DC-5-3h-3 | 0.08359 | |

At 4 hours, 5 ml of the clear aqueous solution was taken out from the remaining clear aqueous solution in the flask. The experiments were done for the 5 ml of the clear aqueous solution taken out at 4 hours using the same protocol as for the 5 ml of the clear aqueous solution taken out at 1 hour. Based on the HPLC data and the measurement data of Example 14, the docetaxel concentrations of the supernatants prepared from solution of DC-5-4h have been calculated and shown in the Table 5. At 4 hours, the docetaxel concentration of the clear aqueous solution after the filtration was about 96.62% of the docetaxel concentration of the clear aqueous solution at 0 hour before the filtration.

TABLE 5

| | Docetaxel | Average Docetaxel |
|---|---|---|
| Solution Number | Concentration (mg/ml) | Concentration (mg/ml) |
| DC-5-4h-1 | 0.08293 | 0.08317 |
| DC-5-4h-2 | 0.08336 | |
| DC-5-4h-3 | 0.08323 | |

At 5 hours, 5 ml of the clear aqueous solution was taken out from the remaining clear aqueous solution in the flask. The experiments were done for the 5 ml of the clear aqueous solution taken out at 5 hours using the same protocol as for the 5 ml of the clear aqueous solution taken out at 1 hour. Based on the HPLC data and the measurement data of Example 14, the docetaxel concentrations of the supernatants prepared from solution of DC-5-5h have been calculated and shown in the Table 6. At 5 hours, the docetaxel concentration of the clear aqueous solution after the filtration was about 94.54% of the docetaxel concentration of the clear aqueous solution at 0 hour before the filtration.

TABLE 6

| | Docetaxel | Average Docetaxel |
|---|---|---|
| Solution Number | Concentration (mg/ml) | Concentration (mg/ml) |
| DC-5-5h-1 | 0.08149 | 0.08138 |
| DC-5-5h-2 | 0.08151 | |
| DC-5-5h-3 | 0.08114 | |

### Example 16: Composition Comprising Docetaxel and Human Serum Albumin (HSA)

The ratio by weight of docetaxel to HSA prepared was about 1:120.

Docetaxel (15 mg) was dissolved in methanol (7.7 ml) in a glass vial to give a clear solution. A solution of HSA (1800 mg, 9 ml) (20% Human Albumin solution for infusion with sodium caprylate (0.140˜0.180 mmol per gram of proteins) as the only stabilizer in the solution) was added into 9 ml of water to give a HSA solution (18 ml) in a round bottom flask. The methanol solution of docetaxel was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting aqueous solution was lyophilized overnight to give a white solid. A sample of 50 mg of the lyophilized solid was reconstituted by adding 1 mL water to give a clear solution. The clear aqueous solution stayed clear without any solid precipitation up to 7 hours at room temperature (about 20˜25)° C. After 8 hours at room temperature (about 20˜25° C.), white precipitation was formed in the aqueous solution. Another sample of 50 mg of the lyophilized solid was reconstituted by adding 1 mL water to give a clear solution. The clear aqueous solution stayed clear without any solid precipitation up to 24 hours in refrigerator (about 2˜5° C.).

### Example 17: Measure the Docetaxel Concentrations in the Clear Aqueous Solutions Before the Filtration at 0 Hour, and after the Filtration at 1 Hour, 2 Hours, 3 Hours, 4 Hours, and 5 Hours

1.6 g of the lyophilized solid of the composition comprising docetaxel and HSA (the ratio by weight about 1:120) from Example 16 was dissolved in 32 ml of water to give a clear aqueous solution in a flask, which was kept at about 20˜25° C. Immediately after the lyophilized solid was dissolved in water, 1 ml of the clear aqueous solution was taken out from the 32 ml solution. Then 1 ml of the solution was named as the solution DC-0-0h. To 200 μl of the solution DC-0-0h was added 800 μl of acetonitrile. The mixture was vortexed for seconds and then centrifuged at 4,000 g for 5 minutes. The supernatants were removed and collected followed by injection on HPLC. The same procedure was repeated 2 more times for the solution DC-0-0h. Based on the HPLC data and the measurement data of Example 14, the docetaxel concentrations of the supernatants prepared from solution of DC-0-0h have been calculated and shown in the Table 7.

TABLE 7

| | Docetaxel | Average Docetaxel |
|---|---|---|
| Solution Number | Concentration (mg/ml) | Concentration (mg/ml) |
| DC-0-0h-1 | 0.07611 | 0.07627 |
| DC-0-0h-2 | 0.07622 | |
| DC-0-0h-3 | 0.07649 | |

At 1 hour, additional 5 ml of the clear aqueous solution was taken out from the remaining clear aqueous solution in the flask. Then 1 ml of the solution was taken out from the 5 ml clear aqueous solution and filtered by a 0.22 micron aqueous phase filter to give the solution DC-1-1h, and the remaining 4 ml of the solution was filtered by the same 0.22 micron aqueous phase filter at 1 ml at a time to give the solutions DC-2-1h, DC-3-1h, DC-4-1h, and DC-5-1h. To 200 μl of the solution DC-5-1h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution DC-5-1h. Based on the HPLC data and the measurement data of Example 14, the docetaxel concentrations of the supernatants prepared from solution of DC-5-1h have been calculated and shown in the Table 8. At 1 hour, the docetaxel concentration of the clear aqueous solution after the filtration was about 99.58% of the docetaxel concentration of the clear aqueous solution at 0 hour before the filtration.

TABLE 8

| | Docetaxel | Average Docetaxel |
|---|---|---|
| Solution Number | Concentration (mg/ml) | Concentration (mg/ml) |
| DC-5-1h-1 | 0.07573 | 0.07595 |
| DC-5-1h-2 | 0.07615 | |
| DC-5-1h-3 | 0.07597 | |

At 2 hours, 5 ml of the clear aqueous solution was taken out from the remaining clear aqueous solution in the flask. The experiments were done for the 5 ml of the clear aqueous solution taken out at 2 hours using the same protocol as for the 5 ml of the clear aqueous solution taken out at 1 hour. Based on the HPLC data and the measurement data of Example 14, the docetaxel concentrations of the supernatants prepared from solution of DC-5-2h have been calculated and shown in the Table 9. At 2 hours, the docetaxel concentration of the clear aqueous solution after the filtration was about 99.86% of the docetaxel concentration of the clear aqueous solution at 0 hour before the filtration.

TABLE 9

| | Docetaxel | Average Docetaxel |
|---|---|---|
| Solution Number | Concentration (mg/ml) | Concentration (mg/ml) |
| DC-5-2h-1 | 0.07610 | 0.07616 |
| DC-5-2h-2 | 0.07620 | |
| DC-5-2h-3 | 0.07618 | |

At 3 hours, 5 ml of the clear aqueous solution was taken out from the remaining clear aqueous solution in the flask. The experiments were done for the 5 ml of the clear aqueous solution taken out at 3 hour using the same protocol as for the 5 ml of the clear aqueous solution taken out at 1 hour. Based on the HPLC data and the measurement data of Example 14, the docetaxel concentrations of the supernatants prepared from solution of DC-5-3h have been calculated and shown in the Table 10. At 3 hour, the docetaxel concentration of the clear aqueous solution after the filtration was about 99.11% of the docetaxel concentration of the clear aqueous solution at 0 hour before the filtration.

TABLE 10

| | Docetaxel | Average Docetaxel |
|---|---|---|
| Solution Number | Concentration (mg/ml) | Concentration (mg/ml) |
| DC-5-3h-1 | 0.07541 | 0.07559 |
| DC-5-3h-2 | 0.07577 | |
| DC-5-3h-3 | 0.07558 | |

At 4 hours, 5 ml of the clear aqueous solution was taken out from the remaining clear aqueous solution in the flask. The experiments were done for the 5 ml of the clear aqueous solution taken out at 4 hour using the same protocol as for the 5 ml of the clear aqueous solution taken out at 1 hour. Based on the HPLC data and the measurement data of Example 14, the docetaxel concentrations of the supernatants prepared from solution of DC-5-4h have been calculated and shown in the Table 11. At 4 hours, the docetaxel concentration of the clear aqueous solution after the filtration was about 99.30% of the docetaxel concentration of the clear aqueous solution at 0 hour before the filtration.

TABLE 11

| | Docetaxel | Average Docetaxel |
|---|---|---|
| Solution Number | Concentration (mg/ml) | Concentration (mg/ml) |
| DC-5-4h-1 | 0.07616 | 0.07573 |
| DC-5-4h-2 | 0.07508 | |
| DC-5-4h-3 | 0.07596 | |

At 5 hours, 5 ml of the clear aqueous solution was taken out from the remaining clear aqueous solution in the flask. The experiments were done for the 5 ml of the clear aqueous solution taken out at 5 hour using the same protocol as for the 5 ml of the clear aqueous solution taken out at 1 hour. Based on the HPLC data and the measurement data of Example 14, the docetaxel concentrations of the supernatants prepared from solution of DC-5-5h have been calculated and shown in the Table 12. At 5 hours, the docetaxel concentration of the clear aqueous solution after the filtration was about 97.84% of the docetaxel concentration of the clear aqueous solution at 0 hour before the filtration.

TABLE 12

| | Docetaxel | Average Docetaxel |
|---|---|---|
| Solution Number | Concentration (mg/ml) | Concentration (mg/ml) |
| DC-5-5h-1 | 0.07495 | 0.07462 |
| DC-5-5h-2 | 0.07449 | |
| DC-5-5h-3 | 0.07442 | |

### Example 18: Measure the Docetaxel Concentrations in the Clear Aqueous Solutions Before the Filtration at 0 Hour, and after the Filtration at 1 Hour, 2 Hours, 3 Hours, 4 Hours, 5 Hours, and 6 Hours

The whole amount of the lyophilized solid of the composition comprising docetaxel and HSA (the ratio by weight about 1:100) from Example 12 (prepared from 15 mg of docetaxel) was dissolved in 45 ml of water to give a clear aqueous solution in a flask, which had a concentration of docetaxel about 0.3333 mg/ml and was kept at about 20˜25° C. Immediately after the lyophilized solid was dissolved in water, 1 ml of the clear aqueous solution was taken out from the 45 ml solution. Then 1 ml of the solution was named as the solution DC-0-0h. To 200 μl of the solution DC-0-0h was added 800 μl of acetonitrile. The mixture was vortexed for seconds and then centrifuged at 4,000 g for 5 minutes. The supernatants were removed and collected followed by injection on HPLC. The same procedure was repeated 2 more times for the solution DC-0-0h. Based on the HPLC data and the measurement data of Example 14, the docetaxel concentrations of the supernatants prepared from solution of DC-0-0h have been calculated and shown in the Table 13.

TABLE 13

| | Docetaxel | Average Docetaxel |
|---|---|---|
| Solution Number | Concentration (mg/ml) | Concentration (mg/ml) |
| DC-0-0h-1 | 0.06494 | 0.06484 |
| DC-0-0h-2 | 0.06455 | |
| DC-0-0h-3 | 0.06502 | |

At 1 hour, additional 5 ml of the clear aqueous solution was taken out from the remaining clear aqueous solution in the flask. Then 1 ml of the solution was taken out from the 5 ml clear aqueous solution and filtered by a 0.22 micron aqueous phase filter to give the solution DC-1-1h, and the remaining 4 ml of the solution was filtered by the same 0.22 micron aqueous phase filter at 1 ml at a time to give the solutions DC-2-1h, DC-3-1h, DC-4-1h, and DC-5-1h. To 200 μl of the solution DC-5-1h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution DC-5-1h. Based on the HPLC data and the measurement data of Example 14, the docetaxel concentrations of the supernatants prepared from solution of DC-5-1h have been calculated and shown in the Table 14. At 1 hour, the docetaxel concentration of the clear aqueous solution after the filtration was about 99.24% of the docetaxel concentration of the clear aqueous solution at 0 hour before the filtration.

TABLE 14

| | Docetaxel | Average Docetaxel |
|---|---|---|
| Solution Number | Concentration (mg/ml) | Concentration (mg/ml) |
| DC-5-1h-1 | 0.06430 | 0.06435 |
| DC-5-1h-2 | 0.06451 | |
| DC-5-1h-3 | 0.06424 | |

At 2 hours, 5 ml of the clear aqueous solution was taken out from the remaining clear aqueous solution in the flask. The experiments were done for the 5 ml of the clear aqueous solution taken out at 2 hours using the same protocol as for the 5 ml of the clear aqueous solution taken out at 1 hour. Based on the HPLC data and the measurement data of Example 14, the docetaxel concentrations of the supernatants prepared from solution of DC-5-2h have been calculated and shown in the Table 15. At 2 hours, the docetaxel concentration of the clear aqueous solution after the filtration was about 98.61% of the docetaxel concentration of the clear aqueous solution at 0 hour before the filtration.

TABLE 15

| | Docetaxel | Average Docetaxel |
|---|---|---|
| Solution Number | Concentration (mg/ml) | Concentration (mg/ml) |
| DC-5-2h-1 | 0.06400 | 0.06394 |
| DC-5-2h-2 | 0.06361 | |
| DC-5-2h-3 | 0.06420 | |

At 3 hours, 5 ml of the clear aqueous solution was taken out from the remaining clear aqueous solution in the flask. The experiments were done for the 5 ml of the clear aqueous solution taken out at 3 hours using the same protocol as for the 5 ml of the clear aqueous solution taken out at 1 hour. Based on the HPLC data and the measurement data of Example 14, the docetaxel concentrations of the supernatants prepared from solution of DC-5-3h have been calculated and shown in the Table 16. At 3 hours, the docetaxel concentration of the clear aqueous solution after the filtration was about 98.69% of the docetaxel concentration of the clear aqueous solution at 0 hour before the filtration.

TABLE 16

| | Docetaxel | Average Docetaxel |
|---|---|---|
| Solution Number | Concentration (mg/ml) | Concentration (mg/ml) |
| DC-5-3h-1 | 0.06396 | 0.06399 |
| DC-5-3h-2 | 0.06411 | |
| DC-5-3h-3 | 0.06390 | |

At 4 hours, 5 ml of the clear aqueous solution was taken out from the remaining clear aqueous solution in the flask. The experiments were done for the 5 ml of the clear aqueous solution taken out at 4 hours using the same protocol as for the 5 ml of the clear aqueous solution taken out at 1 hour. Based on the HPLC data and the measurement data of Example 14, the docetaxel concentrations of the supernatants prepared from solution of DC-5-4h have been calculated and shown in the Table 17. At 4 hours, the docetaxel concentration of the clear aqueous solution after the filtration was about 98.66% of the docetaxel concentration of the clear aqueous solution at 0 hour before the filtration.

TABLE 17

| | Docetaxel | Average Docetaxel |
|---|---|---|
| Solution Number | Concentration (mg/ml) | Concentration (mg/ml) |
| DC-5-4h-1 | 0.06404 | 0.06397 |
| DC-5-4h-2 | 0.06422 | |
| DC-5-4h-3 | 0.06366 | |

At 5 hours, 5 ml of the clear aqueous solution was taken out from the remaining clear aqueous solution in the flask. The experiments were done for the 5 ml of the clear aqueous solution taken out at 5 hours using the same protocol as for the 5 ml of the clear aqueous solution taken out at 1 hour. Based on the HPLC data and the measurement data of Example 14, the docetaxel concentrations of the supernatants prepared from solution of DC-5-5h have been calculated and shown in the Table 18. At 5 hours, the docetaxel concentration of the clear aqueous solution after the filtration was about 98.16% of the docetaxel concentration of the clear aqueous solution at 0 hour before the filtration.

TABLE 18

| | Docetaxel | Average Docetaxel |
|---|---|---|
| Solution Number | Concentration (mg/ml) | Concentration (mg/ml) |
| DC-5-5h-1 | 0.06366 | 0.06365 |
| DC-5-5h-2 | 0.06366 | |
| DC-5-5h-3 | 0.06362 | |

At 6 hours, 5 ml of the clear aqueous solution was taken out from the remaining clear aqueous solution in the flask. The experiments were done for the 5 ml of the clear aqueous solution taken out at 6 hours using the same protocol as for the 5 ml of the clear aqueous solution taken out at 1 hour. Based on the HPLC data and the measurement data of Example 14, the docetaxel concentrations of the supernatants prepared from solution of DC-5-5h have been calculated and shown in the Table 19. At 6 hours, the docetaxel concentration of the clear aqueous solution after the filtration was about 96.90% of the docetaxel concentration of the clear aqueous solution at 0 hour before the filtration.

TABLE 19

| | Docetaxel | Average Docetaxel |
|---|---|---|
| Solution Number | Concentration (mg/ml) | Concentration (mg/ml) |
| DC-5-5h-1 | 0.06262 | 0.06283 |
| DC-5-5h-2 | 0.06287 | |
| DC-5-5h-3 | 0.06300 | |

### Example 19: Measure the Docetaxel Concentrations in the Clear Aqueous Solutions Before the Filtration at 0 Hour, and after the Filtration at 1 Hour, 2 Hours, 3 Hours, and 4 Hours

The whole amount of the lyophilized solid of the composition comprising docetaxel and HSA (the ratio by weight about 1:100) from Example 13 (prepared from 15 mg of docetaxel) was dissolved in 45 ml of water to give a clear aqueous solution in a flask, which had a concentration of docetaxel about 0.3333 mg/ml and was kept at about 20˜25° C. Immediately after the lyophilized solid was dissolved in water, 1 ml of the clear aqueous solution was taken out from the 45 ml solution. Then 1 ml of the solution was named as the solution DC-0-0h. To 200 μl of the solution DC-0-0h was added 800 μl of acetonitrile. The mixture was vortexed for seconds and then centrifuged at 4,000 g for 5 minutes. The supernatants were removed and collected followed by injection on HPLC. The same procedure was repeated 2 more times for the solution DC-0-0h. Based on the HPLC data and the measurement data of Example 14, the docetaxel concentrations of the supernatants prepared from solution of DC-0-0h have been calculated and shown in the Table 20.

TABLE 20

| | Docetaxel | Average Docetaxel |
|---|---|---|
| Solution Number | Concentration (mg/ml) | Concentration (mg/ml) |
| DC-0-0h-1 | 0.06535 | 0.06540 |
| DC-0-0h-2 | 0.06542 | |
| DC-0-0h-3 | 0.06543 | |

At 1 hour, additional 5 ml of the clear aqueous solution was taken out from the remaining clear aqueous solution in the flask. Then 1 ml of the solution was taken out from the 5 ml clear aqueous solution and filtered by a 0.22 micron aqueous phase filter to give the solution DC-1-1h, and the remaining 4 ml of the solution was filtered by the same 0.22 micron aqueous phase filter at 1 ml at a time to give the solutions DC-2-1h, DC-3-1h, DC-4-1h, and DC-5-1h. To 200 μl of the solution DC-5-1h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution DC-5-1h. Based on the HPLC data and the measurement data of Example 14, the docetaxel concentrations of the supernatants prepared from solution of DC-5-1h have been calculated and shown in the Table 21. At 1 hour, the docetaxel concentration of the clear aqueous solution after the filtration was about 99.57% of the docetaxel concentration of the clear aqueous solution at 0 hour before the filtration.

TABLE 21

| | Docetaxel | Average Docetaxel |
|---|---|---|
| Solution Number | Concentration (mg/ml) | Concentration (mg/ml) |
| DC-5-1h-1 | 0.06522 | 0.06512 |
| DC-5-1h-2 | 0.06514 | |
| DC-5-1h-3 | 0.06500 | |

At 2 hours, 5 ml of the clear aqueous solution was taken out from the remaining clear aqueous solution in the flask. The experiments were done for the 5 ml of the clear aqueous solution taken out at 2 hours using the same protocol as for the 5 ml of the clear aqueous solution taken out at 1 hour. Based on the HPLC data and the measurement data of Example 14, the docetaxel concentrations of the supernatants prepared from solution of DC-5-2h have been calculated and shown in the Table 22. At 2 hours, the docetaxel concentration of the clear aqueous solution after the filtration was about 98.29% of the docetaxel concentration of the clear aqueous solution at 0 hour before the filtration.

TABLE 22

| | Docetaxel | Average Docetaxel |
|---|---|---|
| Solution Number | Concentration (mg/ml) | Concentration (mg/ml) |
| DC-5-2h-1 | 0.06447 | 0.06428 |
| DC-5-2h-2 | 0.06404 | |
| DC-5-2h-3 | 0.06433 | |

At 3 hours, 5 ml of the clear aqueous solution was taken out from the remaining clear aqueous solution in the flask. The experiments were done for the 5 ml of the clear aqueous solution taken out at 3 hours using the same protocol as for the 5 ml of the clear aqueous solution taken out at 1 hour. Based on the HPLC data and the measurement data of Example 14, the docetaxel concentrations of the supernatants prepared from solution of DC-5-3h have been calculated and shown in the Table 23. At 3 hours, the docetaxel concentration of the clear aqueous solution after the filtration was about 97.74% of the docetaxel concentration of the clear aqueous solution at 0 hour before the filtration.

TABLE 23

| | Docetaxel | Average Docetaxel |
|---|---|---|
| Solution Number | Concentration (mg/ml) | Concentration (mg/ml) |
| DC-5-3h-1 | 0.06423 | 0.06392 |
| DC-5-3h-2 | 0.06384 | |
| DC-5-3h-3 | 0.06370 | |

At 4 hours, 5 ml of the clear aqueous solution was taken out from the remaining clear aqueous solution in the flask. The experiments were done for the 5 ml of the clear aqueous solution taken out at 4 hours using the same protocol as for the 5 ml of the clear aqueous solution taken out at 1 hour. Based on the HPLC data and the measurement data of Example 14, the docetaxel concentrations of the supernatants prepared from solution of DC-5-4h have been calculated and shown in the Table 24. At 4 hours, the docetaxel concentration of the clear aqueous solution after the filtration was about 82.29% of the docetaxel concentration of the clear aqueous solution at 0 hour before the filtration.

TABLE 24

| | Docetaxel | Average Docetaxel |
|---|---|---|
| Solution Number | Concentration (mg/ml) | Concentration (mg/ml) |
| DC-5-4h-1 | 0.05592 | 0.05382 |
| DC-5-4h-2 | 0.05334 | |
| DC-5-4h-3 | 0.05219 | |

### Example 20: Measure pH Value of the Clear Aqueous Solution of Composition Comprising Docetaxel and Human Serum Albumin (HSA)

350 mg of the lyophilized solid of the composition comprising docetaxel and HSA (the ratio by weight about 1:100) from Example 11 was dissolved in 10 ml of water to give a clear aqueous solution. The clear aqueous solution was kept at about 25° C., and measured for pH value. The pH value of the clear aqueous solution is 7.10 (3 measurements: 7.09, 7.10, and 7.11).

350 mg of the lyophilized solid of the composition comprising docetaxel and HSA (the ratio by weight about 1:100) from Example 11 was dissolved in 10 ml of 0.9% saline solution, which had pH value about 5.41, to give a clear aqueous solution. The clear aqueous solution was kept at about 25° C., and measured for pH value. The pH value of the clear aqueous solution is 6.92 (3 measurements: 6.91, 6.92, and 6.92).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising docetaxel, proteins comprising a human serum albumin, and sodium caprylate, wherein the docetaxel and the human serum albumin in the composition have a ratio by weight from about 1:70 to about 1:300, and wherein the composition does not contain sodium N-acetyltryptophanate.

2. The composition of claim 1, wherein the docetaxel and the human serum albumin in the composition are in a ratio by weight from about 1:80 to about 1:150.

3. The composition of any one of claims 1-2, wherein the human serum albumin is a native human serum albumin.

4. The composition of any one of claims 1-3, wherein the ratio of the amount of sodium caprylate to the amount of the proteins in the composition is from about 0.140 mmol to about 0.180 mmol of sodium caprylate per one gram of the proteins.

5. The composition of any one of claims 1-4, wherein the composition is a solid formulation.

6. The composition of any one of claims 1-5, wherein the composition is an aqueous formulation.

7. The composition of any one of claims 1-5, wherein the aqueous formulation is substantially free of solvent other than water.

8. The composition of any one of claims 6-7, wherein the aqueous formulation is a clear aqueous solution.

9. The composition of any one of claims 6-8, wherein the aqueous formulation is a clear aqueous solution for at least 1 hours, at least 2 hours, at least 3 hours, at least 4 hours, at least 5 hours, at least 6 hours, at least 8 hours, or at least 24 hours.

10. A composition comprising docetaxel, proteins comprising a human serum albumin, and N-acetyltryptophanate, wherein the docetaxel and the human serum albumin in the composition have a ratio by weight from about 1:80 to less than 1:150.

11. The composition of claim 10, wherein the docetaxel and the human serum albumin in the composition are in a ratio by weight from about 1:90 to about 1:140.

12. The composition of any one of claims 10-11, wherein the human serum albumin is a native human serum albumin.

13. The composition of any one of claims 10-12, wherein the composition comprises from about 0.064 mmol to about 0.096 mmol sodium N-acetyltryptophanate and from about 0.064 mmol to about 0.096 mmol sodium caprylate per gram of the proteins in the composition.

14. The composition of any one of claims 10-13, wherein the composition is a solid formulation.

15. The composition of any one of claims 10-13, wherein the composition is an aqueous formulation.

16. The composition of claim 15, wherein the aqueous formulation is substantially free of solvent other than water.

17. The composition of any one of claims 15-16, wherein the aqueous formulation is a clear aqueous solution.

18. The composition of any one of claims 15-17, wherein the aqueous formulation is a clear aqueous solution for at least 1 hours, at least 2 hours, at least 3 hours, at least 4 hours, at least 5 hours, at least 6 hours, at least 8 hours, or at least 24 hours.

19. A pharmaceutical composition comprising the composition of any one of claims 1-18, and a pharmaceutically acceptable carrier.

20. A method of treating a cancer, the method comprising the step of administering to a subject in need thereof a therapeutically effective amount of a pharmaceutical composition of claim 19.

21. The method of claim 20, wherein the cancer is selected from the group consisting of breast cancer, non-small cell lung cancer, prostate cancer, gastric cancer, head and neck cancer, ovarian cancer, pancreatic cancer, and Kaposi's sarcoma.
